(19) United States
(12) Patent Application Publication
(10) Pub. No.: US 2026/0242405 A1
(43) Pub. Date: Aug. 20, 2026

(54) N-DERIVATIZED SIALIC ACIDS AND RELATED SIALOSIDES

(71) Applicant: THE REGENTS OF THE UNIVERSITY OF CALIFORNIA, Oakland, CA (US)

(72) Inventors: Xi CHEN, Oakland, CA (US); Yue YUAN, Oakland, CA (US); Hai YU, Oakland, CA (US); Bijoyananda MISHRA, Oakland, CA (US)

(73) Assignee: THE REGENTS OF THE UNIVERSITY OF CALIFORNIA, Oakland, CA (US)

(21) Appl. No.: 19/656,778

(22) Filed: Apr. 23, 2026

Related U.S. Application Data
(63) Continuation of application No. PCT/IB2024/060449, filed on Oct. 23, 2024.
(60) Provisional application No. 63/592,356, filed on Oct. 23, 2023.

(51) Int. Cl. C07H 15/26; A61K 31/7012; A61K 31/7048; A61K 31/7052; C07H 15/203; C07H 15/22; C07H 17/075

(52) U.S. Cl. CPC C07H 15/26; A61K 31/7012; A61K 31/7048; A61K 31/7052; C07H 15/203; C07H 15/22; C07H 17/075

(57) ABSTRACT

Described herein are 4-N-derivatized sialic acid compounds and efficient chemoenzymatic methods for synthesizing sialosides containing the 4-N-derivatized sialic acid. Methods of using the 4-N-acyl sialic acids and sialosides are also described herein. Described also herein are 8-N-derivatized sialic acid compounds and methods for synthesizing and using the same.

### RELATED APPLICATIONS

This application is a continuation under 35 U.S.C. 111 (a) of International Application No. PCT/IB2024/060449 filed Oct. 23, 2024, published as WO 2025/088529 on May 1, 2025, which claims priority from U.S. Provisional Application No. 63/592,356 filed Oct. 23, 2023, which applications and publication are incorporated herein by reference.

### STATEMENT REGARDING FEDERALLY FUNDED RESEARCH

This invention was made with government support under Grant No. R01AI130684 awarded by the National Institutes of Health (NIH). The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 23, 2024, is entitled “081906-1460518-254110PC_ST26.xml”, and is 19,777 bytes in size.

### BACKGROUND

Sialic acids (Sias) are biologically important but synthetically challenging molecules. They are commonly found at the terminal positions of the carbohydrate components on cell surfaces. With or without additional structural modifications, sialic acid-containing molecules play important roles in processes including intercellular adhesion, carcinoma metastasis, hemostasis and inflammation, bacterial and viral attachment and infection, and other molecular recognition events.

More than 50 different sialic acid structures have been found in nature. N-Acetylneuraminic acid (Neu5Ac, 1) (FIG. 1) is the most abundant form which also provides a basic scaffold for additional naturally occurring structural modifications. O-Acetylation, as one of the most common modifications on sialic acids, is expressed in vertebrates in a tissue and species-specific manner. For instance, 9-O-acetylated and 7,9-di-O-acetylated Neu5Ac are commonly found in mice and humans, while 4-O-acetylated Neu5Ac (Neu4,5Ac₂, 2 of FIG. 1) has been found on glycoconjugates in horses and guinea pigs, but not in humans or pigs. Mouse hepatitis virus strain A (MHV-S) hemagglutinin-esterase (HE) specifically recognizes Neu4,5Ac₂-containing sialosides that are completely resistant to the cleavage by most sialidases, except for influenza virus neuraminidases. Nonetheless, O-acetyl modification of carbohydrates is labile and susceptible to base-catalyzed hydrolysis, and cleavage by selected esterases (such as MHV-S esterase). The detailed biological functions of 4-O-acetylation of sialic acids remain to be elucidated.

### SUMMARY

Described herein are compounds, or pharmaceutically acceptable salts thereof, according to Formula I:

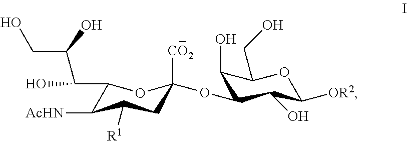

wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, and ProNH-PEG4-biotin.

Also provided herein are compounds, or pharmaceutically acceptable salts thereof, according to Formula II:

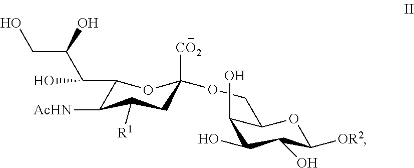

wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcαProNHCbz, 3GalNAcαProNHCbz, 3GalNAcβProNHCbz, ProNH-Biotin, and ProNH-PEG4-biotin.

Also provided herein are compounds, or pharmaceutically acceptable salts thereof, according to Formula III:

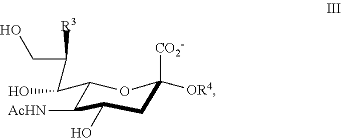

wherein R³ is selected from the group consisting of NHAc, NH₂, and N₃; and R⁴ is selected from the group consisting of H, C₁-C₆ alkyl, C₂-C₆ alkenyl, C₂-C₆ alkynyl, 4-methylumbelliferone (MU), para-nitrophenyl (pNP), indole, a monosaccharide, an oligosaccharide, a glycolipid, a glycopeptide, a glycoprotein, a galactoside, GalβMU, GalβpNP, or Galβ1-4GlcNAcβOR. Also provided herein are methods for inhibiting neuraminidases, and methods and kits for determining the presence of neuraminidases and isolating and purifying neuraminidases and influenza viruses.

The details of one or more embodiments are set forth in the drawings and the description below. Other features, objects, and advantages will be apparent from the description and drawings, and from the claims.

### BRIEF DESCRIPTION OF THE DRAWINGS

FIG. 1 shows examples of compounds described herein.

FIG. 2 shows examples of compounds described herein.

FIG. 3 shows examples of compounds described herein.

FIG. 4, panel (a) shows plots of substrate specificity studies described herein. FIG. 4, panel (b) shows plots of substrate specificity studies described herein.

FIG. 5, panel (a), shows plots of substrate specificity studies described herein; FIG. 5, panel (b), shows BLI assay studies of influenza neuraminidases described herein; FIG. 5, panel (c), shows K_{D} values determined by BLI assays described herein.

FIG. 6, panel (a), shows binding studies of influenza neuraminidases using ELISA studies described herein; FIG. 6, panel (b), shows virus isolation described herein; FIG. 6, panel (c), shows an SDS-PAGE study described herein; FIG. 6, panel (d), shows a polydispersity index study described herein.

FIG. 7 shows compounds described herein.

FIG. 8 shows compounds described herein.

FIG. 9 shows BLI assay studies described herein.

FIG. 10 shows binding studies described herein.

FIG. 11 shows substrate specificity studies described herein.

### DETAILED DESCRIPTION

### I. General

Neuraminidases (NAs) or sialidases are a family of enzymes that catalyze the cleavage of sialic acids from sialosides. NAs generally exist not only in humans but also in bacteria and viruses. Due to continuous threats of epidemics and pandemics caused by influenza A virus (IAV) infections and influenza B virus (IBV) infections, the development of related vaccines and therapeutics are of great interest. NAs of IAV and IBV are significant glycoproteins on viral surfaces that are essential for viral infection and are underexplored candidates for vaccine development. NAs from different sources have shown different preferences toward sialyl linkages and sialic acid forms, and Neu4,5Ac₂-containing sialosides have been shown to be selective substrates for influenza viruses but not for human or bacterial sialidases. Compound 3 of FIG. 1 shows a more stable N-acetyl analog 4NAcNeu5Ac (3).

Due to the key roles of NAs in influenza virus infection, they have been attractive targets in the development of inhibitors and antimicrobial therapeutics. Sialidase inhibitors compete against NA binding to sialosides in a host and have been applied frequently in the treatment of influenza virus infections. Naturally existing sialidase inhibitors and their derivatives have been discovered with the help of virtual screening and molecular docking efforts. Other sialidase inhibitors were designed based on the catalytic mechanisms, loop flexibility, and product analogs. The most potent influenza virus neuraminidase (NA) inhibitors so far were designed based on sialidase transition state analog 2,3-dehydro-N-acetylneuraminic acid (Neu5Ac2en, 4 of FIG. 1) such as 4-amino-2,3-dehydro-N-acetylneuraminic acid (4NH₂Neu5 Ac2en, 5 of FIG. 1) and 4-guanidino-2,3-dehydro-N-acetylneuraminic acid (4-guanidino-Neu5Ac2en or Zanamivir, 6 of FIG. 1).

However, it is unclear whether sialosides containing 4-azido-Neu5Ac (4N₃Neu5Ac, 7 of FIG. 1), 4NH₂Neu5Ac (8 of FIG. 1), or 4-guanidino-Neu5Ac (9 of FIG. 1) are suitable substrates for influenza virus NAs.

Herein efficient chemoenzymatic strategies are described for the synthesis of sialosides containing a diverse array of 4-N-derivatized Neu5Ac. Several 4-N-derivatives of 2,3-dehydro-N-acetylneuraminic acid have also been chemically synthesized. The obtained sialosides as described herein are useful as substrates for substrate specificity studies of sialidases from human, bacterial and influenza A and B virus NAs. The sialosides are also useful, together with 4-N-derivatives of 2,3-dehydro-N-acetylneuraminic acid, as potential inhibitors against a recombinant human sialidase hNEU2 and viral NAs. The compounds and methods described herein are useful for diagnostic tools and therapeutics against influenza A and B viruses.

Herein, efficient chemoenzymatic methods for synthesizing new 4-N-derivatized sialic acids and related sialosides are described. Applications in sialidase substrate specificity and inhibition studies are also described.

Described herein are 4-N-derivatized sialic acid compounds and convenient chemoenzymatic methods for synthesizing the 4-N-derivatized sialic acid and sialosides containing them. Applications of 4-N-derivatized sialic acid compounds and sialosides containing them in sialidase substrate specificity and inhibition studies are also described.

In certain examples, one-pot two-enzyme (OP2E) sialylation systems are used for efficient, high-yield production of a library of 4NH₂Neu5 Ac-containing α2-3- and α2-6-linked sialosides with diverse underlying glycans from chemically synthesized 4-amino-4-deoxy-N-acetylneuraminic acid (4NH₂Neu5Ac). Among these, the use of para-nitrophenylated α2-3- and α2-6-linked sialyl galactosides is demonstrated in high-throughput substrate specificity studies of fifteen different sialidases including those from human, bacteria, influenza A viruses (IAVs), and influenza B viruses (IBVs).

Also described herein are 8-N-derivatized sialic acid compounds and efficient chemoenzymatic methods for synthesizing sialic acid compounds and sialosides containing them, including, but not limited to, para-nitrophenol-tagged α2-3- and α2-6-linked sialyl galactosides containing C8-acetamido, C8-azido, or C8-amino derivatized N-acetylneuraminic acid (Neu5Ac). The 8-N-derivatized sialic acid compounds described herein, optionally when used with other sialosides, can be useful for diagnostic profiling of disease causing sialidase-producing pathogens.

### II. Definitions

As used herein, the term “monosaccharide” refers to a sugar having a six-membered carbon backbone (i.e., a hexose) or a nine-membered carbon backbone (i.e., a nonose). Examples of monosaccharides include, but are not limited to, glucose (Glc), galactose (Gal), mannose (Man), glucuronic acid (GlcA), iduronic acid (IdoA), N-acetylneuraminic acid (Neu5Ac), other sialic acids (Sias), and their derivatives. Monosaccharides also include hexoses with hydroxyl groups substituted with hydrogens, oxo groups, amino groups, acetamido groups, and other functional groups. “Deoxy” monosaccharides refer to monosaccharides having one or more carbon atoms in the hexose backbone having only hydrogen substituents. Monosaccharides also include, but are not limited to, glucosamine (2-amino-2-deoxy-glucose; GlcNH₂), N-acetylglucosamine (2-acetamido-2-deoxy-glucose; GlcNAc), galactosamine (2-amino-2-deoxy-galactose; GalNH₂;), N-acetylgalactosamine (2-acetamido-2-deoxy-galactose; GalNAc), mannosamine (2-amino-2-deoxy-mannose; ManNH2), and N-acetylmannosamine (2-acetamido-2-deoxy-mannose; ManNAc).

As used herein, the term “oligosaccharide” refers to a compound containing at least two sugars covalently linked together. Oligosaccharides include disaccharides, trisaccharides, tetrasaccharides, pentasaccharides, hexasaccharides, heptasaccharides, octasaccharides, and the like. Covalent linkages for linking sugars generally consist of glycosidic linkages (i.e., C—O—C bonds) formed from the hydroxyl groups of adjacent sugars. Linkages can occur between the 1-carbon (the anomeric carbon) and the 4-carbon of adjacent sugars (i.e., a 1-4 linkage), the 1-carbon (the anomeric carbon) and the 3-carbon of adjacent sugars (i.e., a 1-3 linkage), the 1-carbon (the anomeric carbon) and the 6-carbon of adjacent sugars (i.e., a 1-6 linkage), or the 1-carbon (the anomeric carbon) and the 2-carbon of adjacent sugars (i.e., a 1-2 linkage). A sugar can be linked within an oligosaccharide such that the anomeric carbon is in the α- or β-configuration. The oligosaccharides prepared according to the methods described herein can also include linkages between carbon atoms other than the 1-, 2-, 3-, 4-, and 6-carbons. For example, linkages can occur between the 2-carbon and the 3-carbon of adjacent sugars (i.e., a 2-3 linkage), between the 2-carbon and the 6-carbon of adjacent sugars (i.e., a 2-6 linkage), or between the 2-carbon and the 8-carbon of adjacent sugars (i.e., a 2-8 linkage).

As used herein, the term “isomer” refers to a compound having the same bond structure as a reference compound but having a different three-dimensional arrangement of the bonds. An isomer can be, for example, an enantiomer or a diastereomer.

As used herein, the term “glycoside” refers to a saccharide compound having a moiety “—OR” replacing a hydroxyl group of the parent compound, wherein R is another saccharide (e.g., a monosaccharide, oligosaccharide, or polysaccharide) or a non-saccharide moiety (e.g., a lipid, a protein, a peptide, a linker moiety, a label moiety, etc.). In some examples, the moiety-OR in the glycoside replaces the hydroxyl component of the hemiacetal group of the anomeric carbon at the reducing end of the parent saccharide.

A “galactoside” refers to a galactopyranose moiety or a galactofuranose moiety wherein one of the hydroxyl components of the hemiacetal group of the anomeric carbon at the reducing end of the parent compound is replaced with a moiety-OR as described above. Galactosides include, for example, lactosides (i.e., β-D-galactopyranosyl-(1→4)-D-glucopyranoses).

An “N-acetylgalactosaminide” refers to a galactopyranose moiety or a galactofuranose moiety wherein the hydroxyl component of the hemiacetal group of the anomeric carbon at the reducing end of the parent compound is replaced with a moiety-OR as described above, and wherein the hydroxyl group at the carbon-2 of the parent compound is replaced with an N-acetamido group. N-Acetylgalactosaminides include, for example, N-acetylgalactosamine (GalNAc)-derived 2-acetamido-2-deoxy-D-galactopyranosides.

A “glucoside” refers to a glucopyranose moiety or a glucofuranose moiety wherein the hydroxyl component of the hemiacetal group of the anomeric carbon at the reducing end of the parent compound is replaced with a moiety-OR as described above.

An “N-acetylglucosaminide” refers to a glucopyranose moiety or a glucofuranose moiety wherein the hydroxyl component of the hemiacetal group of the anomeric carbon at the reducing end of the parent compound is replaced with a moiety-OR as described above, and wherein the hydroxyl group at the carbon-2 of the parent compound is replaced with an N-acetamido group. N-Acetylglucosaminides include, for example, N-acetylglucosamine (GlcNAc)-derived 2-acetamido-2-deoxy-D-glucopyranosides.

A “sialoside” refers to a sialic acid moiety wherein the hydroxyl component of the hemiacetal group of the anomeric carbon at the reducing end of the parent compound is replaced with a moiety-OR as described above. Sialic acid is a general term for N- and O-substituted derivatives of neuraminic acid or 2-nonulosonic acid, and includes, but is not limited to, N-acetyl (Neu5Ac) or N-glycolyl (Neu5Gc) substitutions, as well as O-substitutions including acetyl, lactyl, methyl, sulfate and phosphate, among others.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C_{4- 6} and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. The term “alkylene” refers to a divalent alkyl radical, wherein the two points of attachment to the diradical are on the same carbon atom or different carbon atoms. As used herein, the term “alkenyl” refers to a straight or branched, unsaturated, aliphatic radical, that contains at least one double bond, having the number of carbon atoms indicated. Alkenyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. As used herein, the term “alkynyl” refers to a straight or branched, unsaturated, aliphatic radical, that contains at least one double bond, having the number of carbon atoms indicated. Alkenyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆.

As used herein, the terms “halo” and “halogen” refer to a fluorine, chlorine, bromine, or iodine atom.

As used herein, the term “CMP-sialic acid synthetase” refers to a polypeptide that catalyzes the synthesis of cytidine monophosphate sialic acid (CMP-sialic acid) from cytidine triphosphate (CTP) and sialic acid.

As used herein, the term “sialyltransferase” refers to an enzyme that catalyzes the transfer of a sialic acid to a monosaccharide, an oligosaccharide, or another glycosylated molecule.

As used herein, the term “sialidase” or “neuraminidase” refers to an enzyme that catalyzes the cleavage of a terminal sialic acid from a sialylated target such as an oligosaccharide, a glycosylated compound, a polysaccharide, or a glycosylated protein.

The term “variant,” in the context of the enzymes in the present disclosure, means a polypeptide, typically recombinant, that comprises one or more amino acid substitutions relative to a corresponding, naturally-occurring or unmodified sialyltransferase.

As used herein, the term “selective substrate” refers to a compound that selectively binds to any portion of the target and undergoes a transformation catalyzed by the target (e.g., an influenza neuraminidase).

As used herein, the term “selectively binds” refers to the ability of a selective binding compound to bind to a target with greater affinity than it binds to a non-target. In certain embodiments, specific binding refers to binding to a target with an affinity that is at least 2, 5, 10, 25, 50, 75, 100, 150, 200, 250, 500, 1000 or more times greater than the affinity for a non-target.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

The terms “amino acid modification” and “amino acid alteration” refer to a substitution, a deletion, or an insertion of one or more amino acids. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some examples, an amino acid with an acidic side chain, e.g., E or D, may be substituted with its uncharged counterpart, e.g., Q or N, respectively; or vice versa. Each of the following eight groups contains exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

In certain examples, an enzyme variant will have at least about 80%, e.g., at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, sequence identity to any one of the amino acid sequences set forth herein.

In some examples, the polypeptide further comprises one or more heterologous amino acid sequences located at the N-terminus and/or the C-terminus of the polypeptide. The polypeptide can contain a number of heterologous sequences that are useful for expressing, purifying, and/or using the polypeptide. The polypeptide can contain, for example, a poly-histidine tag (e.g., a His tag); a calmodulin-binding peptide (CBP) tag; a NorpA peptide tag; a Strep tag (e.g., Trp-Ser-His-Pro-Gln-Phe-Glu-Lys) for recognition by/binding to streptavidin or a variant thereof; a FLAG peptide (i.e., Asp-Tyr-Lys-Asp-Asp-Asp-Asp-Lys) for recognition by/binding to anti-FLAG antibodies (e.g., MI, M2, M5); a glutathione S-transferase (GST); or a maltose binding protein (MBP) polypeptide.

As used herein, the term “forming a reaction mixture” refers to the process of bringing into contact at least two distinct species such that they mix together and can react, either modifying one of the initial reactants or forming a third distinct species, i.e., a product. It should be appreciated, however, the resulting reaction product can be produced directly from a reaction between the added reagents or from an intermediate from one or more of the added reagents which can be produced in the reaction mixture.

### III. Sialosides Containing a 4-N-Derivatized Sialic Acid

The compounds described herein can be useful as substrates and/or inhibitors to neuraminidases. In some examples, the compounds inhibit influenza A virus neuraminidases and/or influenza B virus neuraminidases. In some examples, the compounds selectively inhibit influenza A virus neuraminidases. In some examples, the compounds selectively inhibit influenza B virus neuraminidases.

Accordingly, provided herein are compounds according to Formula I:

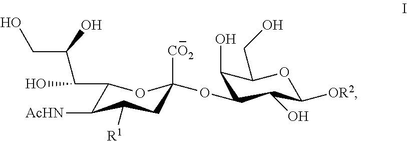

or a pharmaceutically acceptable salt thereof, wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, and ProNH-PEG4-biotin.

In some examples, the compound is a compound according to Formula I (a):

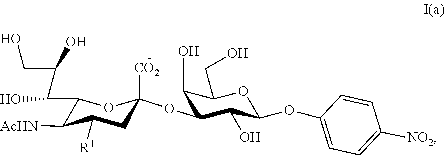

or a pharmaceutically acceptable salt thereof, wherein R¹ is selected from the group consisting of NH₂, NHAc, guanidine, and N₃. In some examples, R¹ is NH₂. In some examples, R¹ is guanidine. In some examples, R¹ is N₃.

In some examples, the compound is a compound according to Formula I (b):

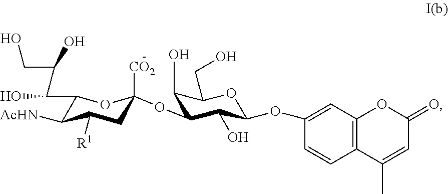

or a pharmaceutically acceptable salt thereof, wherein R¹ is selected from the group consisting of NH₂, NHAc, guanidine, and N₃. In some examples, R¹ is NH₂. In some examples, R¹ is guanidine. In some examples, R¹ is N₃.

Also provided herein is a compound according to Formula II:

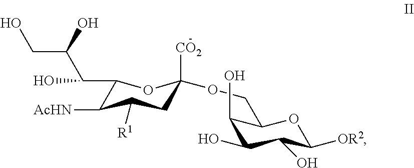

or a pharmaceutically acceptable salt thereof, wherein, R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, ProNH-Biotin, and ProNH-PEG4-biotin.

In some examples, the compound is a compound according to Formula II (a),

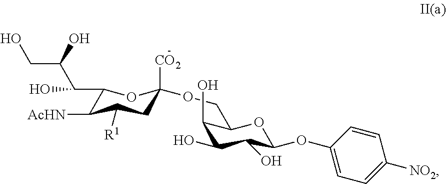

or a pharmaceutically acceptable salt thereof, wherein R¹ is selected from the group consisting of NH₂, NHAc, guanidine, and N₃. In some examples, R¹ is NH₂. In some examples, R¹ is guanidine. In some examples, R¹ is N₃.

In some examples, the compound is a compound according to Formula II (b),

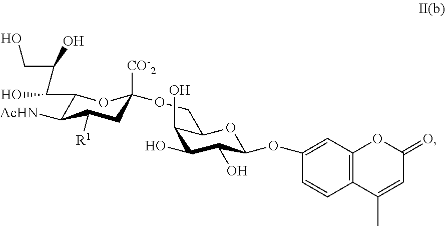

or a pharmaceutically acceptable salt thereof, wherein R¹ is selected from the group consisting of NH₂, NHAc, guanidine, and N₃. In some examples, R¹ is NH₂. In some examples, R¹ is guanidine. In some examples, R¹ is N₃.

In some examples, the compound is a biotinylated compound selected from the group consisting of

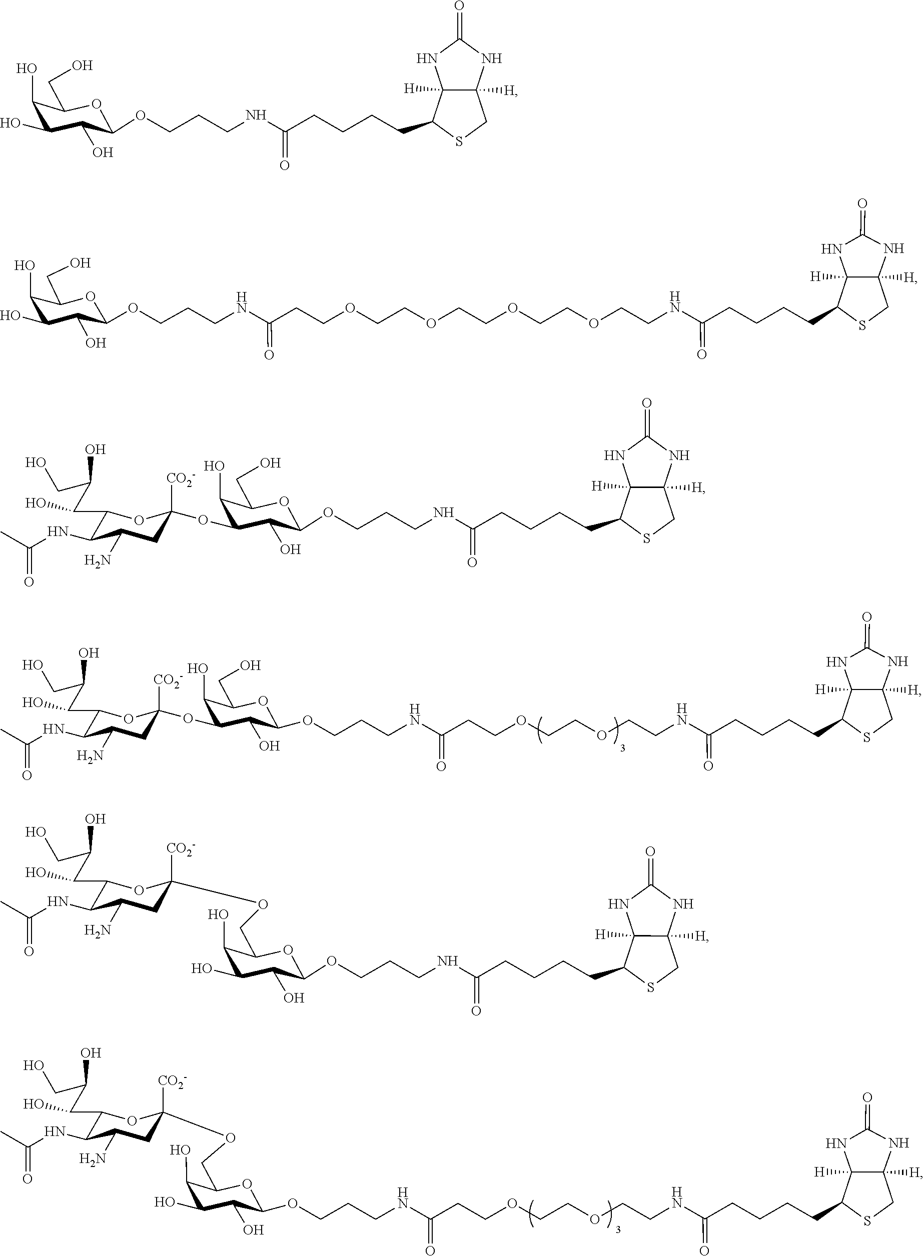

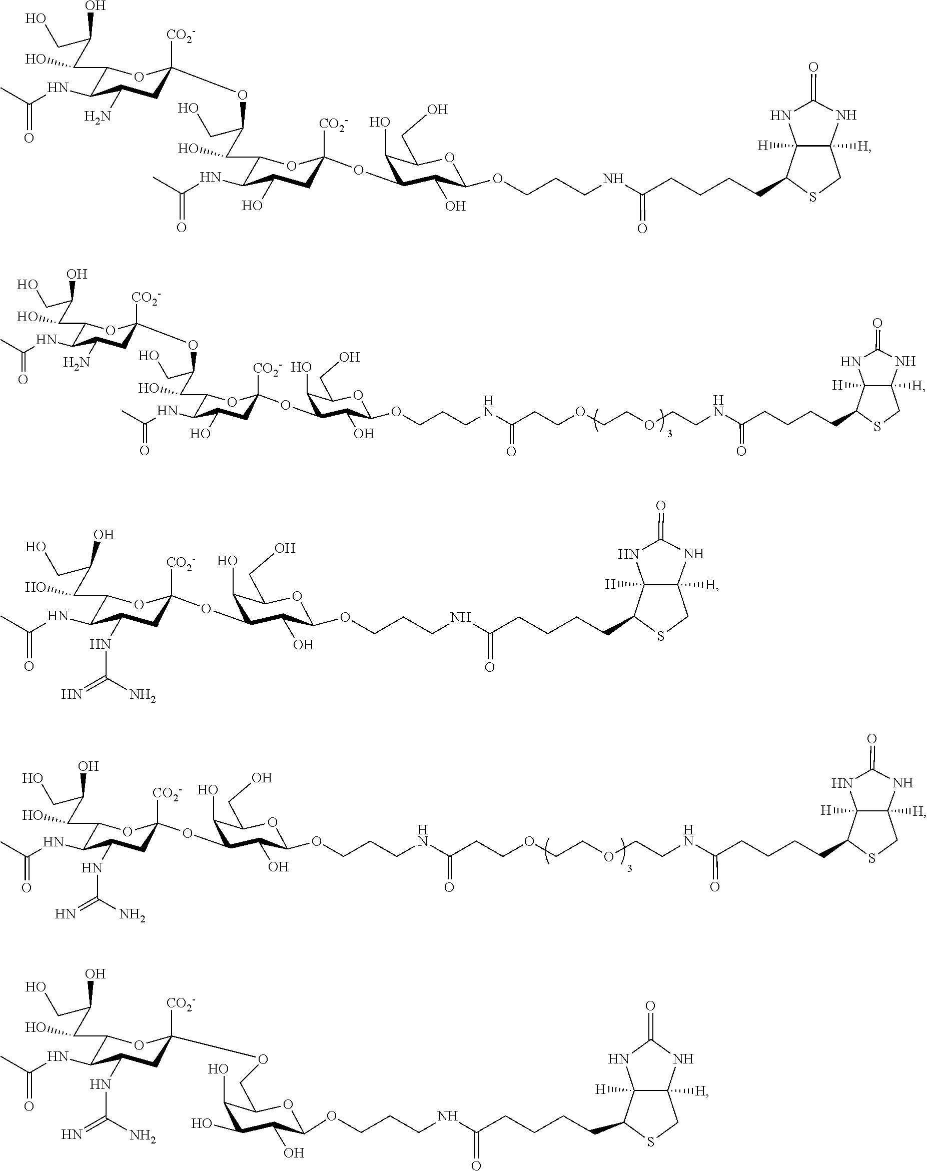

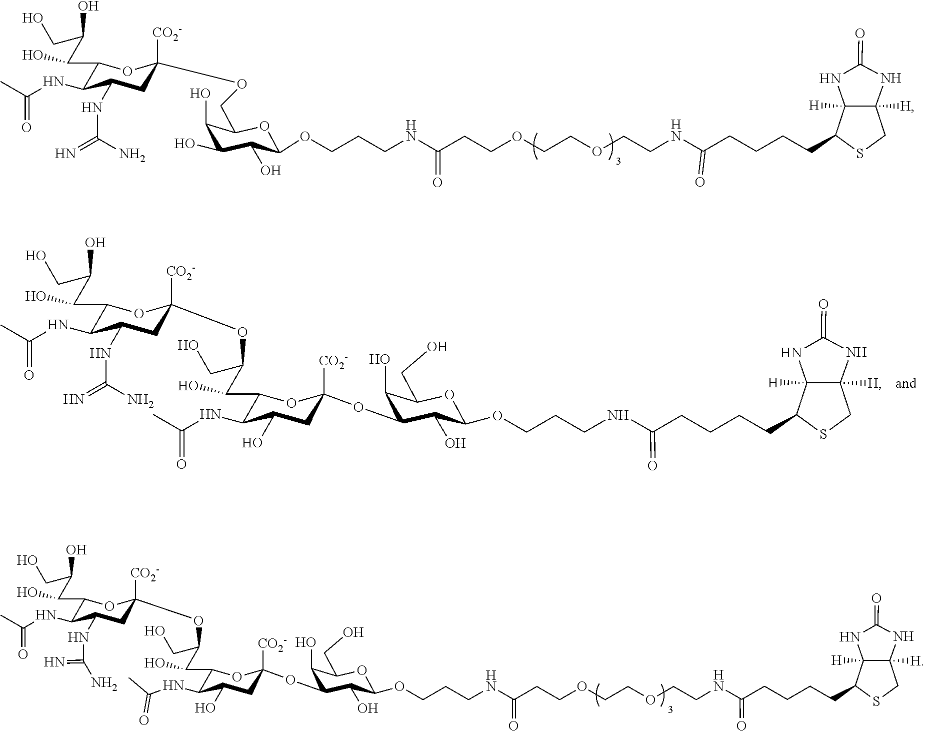

In some examples, the compound is an α2-3-linked 4NH₂Neu5Ac-sialoside selected from the group consisting of

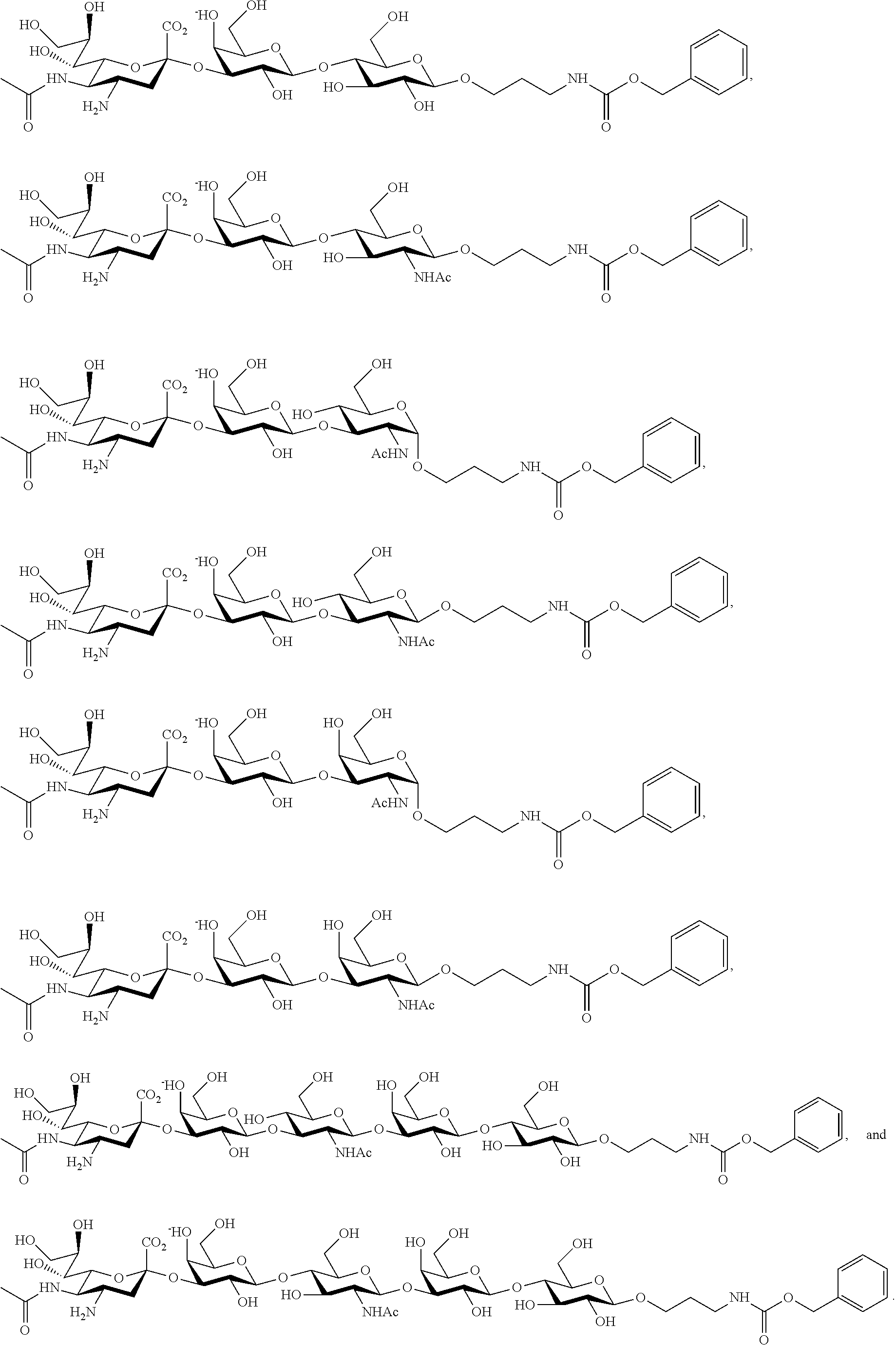

In some examples, the compound is an α2-6-linked 4NH₂Neu5Ac-sialoside selected from the group consisting of:

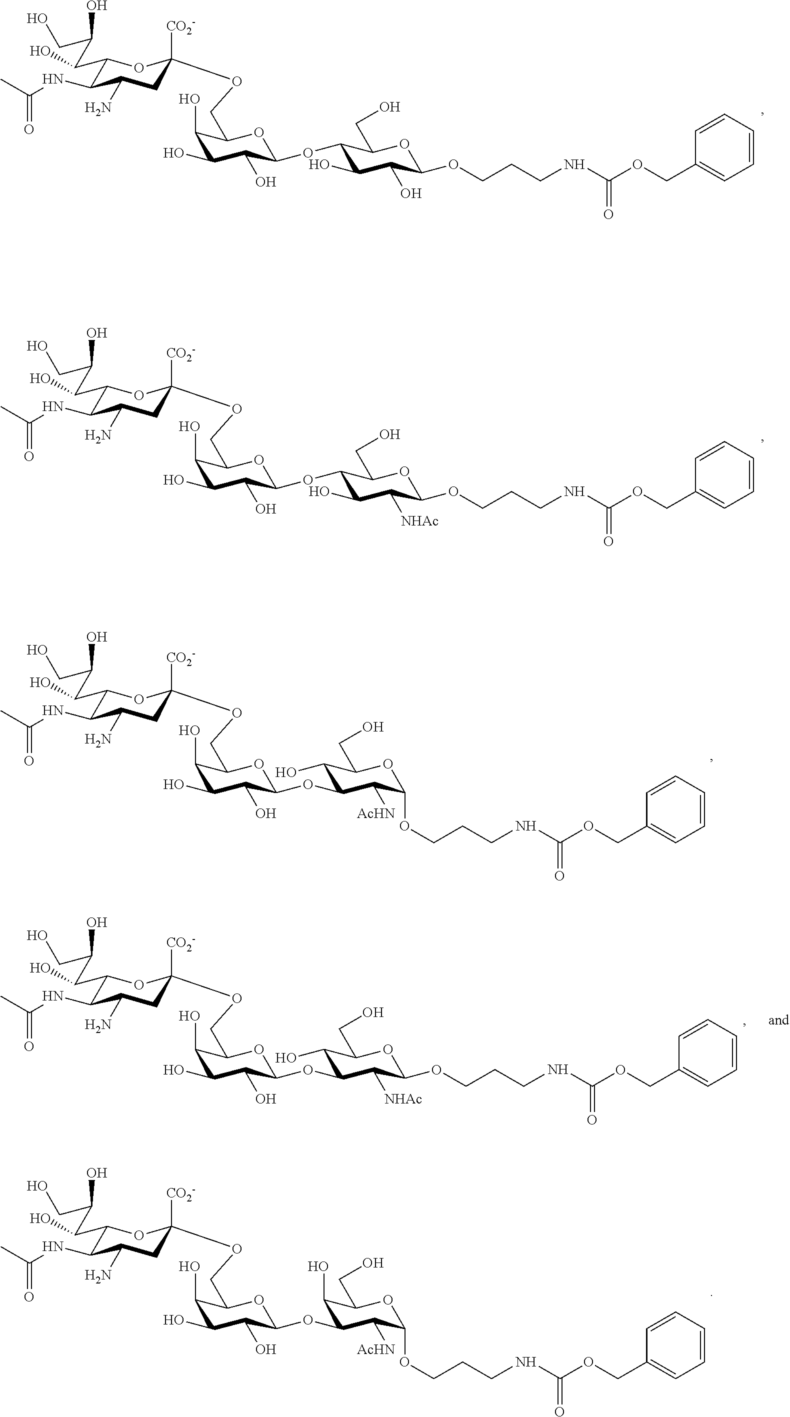

In some examples, the compound is a 4-methylumbelliferyl (MU)-tagged sialoside selected from the group consisting of:

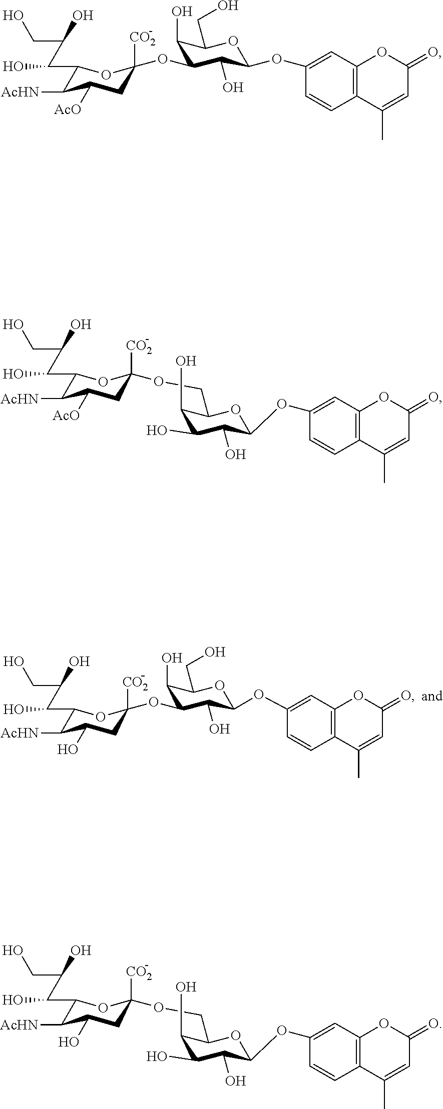

### IV. Chemoenzymatic Synthesis of Sialosides Containing 4-N-Derivatized Sialic Acid

Also provided herein are methods of making the compounds described herein.

While a number of exemplary protecting groups are set forth herein, one of skill in the art will appreciate that still other protecting strategies can be employed in the methods for the synthesis of particular sugars and/or glycosides. Such protecting groups are described, for example, in Wuts, Greene's Protective Groups in Organic Synthesis, 5^{th} Ed., Wiley & Sons, 2014.

Any suitable CMP-sialic acid synthetase (i.e., N-acylneuraminate cytidylyltransferase, EC 2.7.7.43, also referred to as “CSS”) can be used in the methods for forming the sialosides disclosed herein. For example, CMP-sialic acid synthetases from E. coli, C. thermocellum, S. agalactiae, or N. meningitidis can be used. In some examples, the CMP sialic acid synthetase is Neisseria meningitidis CMP-sialic acid synthetase (NmCSS).

In some examples, the CMP-sialic acid synthetase is NmCSS (NCBI Accession No. WP_025459740.1) or a catalytically active variant thereof. In some examples, the CMP-sialic acid synthetase comprises the polypeptide sequence:

(SEQ ID NO: 1)
EKQNIAVILARQNSKGLPLKNLRKMNGISLLGHTINAAISSKCFD
RIIVSTDGGLIAEEAKNFGVEVVLRPAELASDTASSISGVIHALE
TIGSNSGTVTLLQPTSPLRTGAHIREAFSLFDEKIKGSVVSACPM
EHHPLKTLLQINNGEYAPMRHLSDLEQPRQQLPQAFRPNGAIYIN
DTASLIANNCFFIAPTKLYIMSHQDSIDIDTELDLQQAENILHHK
ES,
or a catalytically
active variant thereof.

Any suitable sialyltransferase (also referred to as “ST”) can be used in the methods for forming the sialosides disclosed herein. In some examples, the sialyltransferase is a beta-galactoside alpha-2,3-sialyltransferases belonging to Glycosyltransferase family 80 (GT80 using CAZy nomenclature), which catalyzes the following conversion: CMP-sialic acid+β-D-galactosyl-R=CMP+α-sialic acid-(2→3)-β-D-galactosyl-R, where the acceptor is GalβOR, where R is H, a monosaccharide, an oligosaccharide, a polysaccharide, a glycopeptide, a glycoprotein, a glycolipid, or a hydroxyl-containing compound. GT80 family sialyltransferases also include galactoside or N-acetylgalactosaminide alpha-2,6-sialyltransferases that catalyze the following conversion: CMP-sialic acid+galactosyl/GalNAc-R=CMP+α-sialic acid-(2→6)-β-D-galactosyl/GalNAc-R, where the acceptor is GalOR or GalNAcOR, where R is H, serine or threonine on a peptide or protein, a monosaccharide, an oligosaccharide, a polysaccharide, a glycopeptide, a glycoprotein, a glycolipid, or a hydroxyl-containing compound.

Sialyltransferases in family EC 2.4.99, such as beta-galactosaminide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8), and lactosylceramide alpha-2,3-sialyltransferase (EC 2.4.99.9), can also be used in the methods for preparing sialosides.

In some examples, the sialyltransferase is selected from the group consisting of PmST1, a PmST1 variant, PmST2, a PmST2 variant, PmST3, a PmST3 variant, Pd2, 6ST, Psp2, 6ST, CstI, CstII, and a polysialyltransferase.

In some examples, the sialyltransferase is PmST1 (NCBI Accession No. WP_005753497.1) or a catalytically active variant thereof. In some examples, the sialyltransferase is PmST1_M144D or a catalytically active variant thereof. In some examples, the sialyltransferase comprises the polypeptide sequence:

(SEQ ID NO: 2)
KTITLYLDPASLPALNQLMDFTQNNEDKTHPRIFGLSRFKIPDNI
ITQYQNIHFVELKDNRPTEALFTILDQYPGNIELNIHLNIAHSVQ
LIRPILAYRFKHLDRVSIQQLNLYDDGSDEYVDLEKEENKDISAE
IKQAEKQLSHYLLTGKIKFDNPTIARYVWQSAFPVKYHELSTDYF
EKAEFLQPLKEYLAENYQKMDWTAYQQLTPEQQAFYLTLVGFNDE
VKQSLEVQQAKFIFTGTTTWEGNTDVREYYAQQQLNLLNHFTQAE
GDLFIGDHYKIYFKGHPRGGEINDYILNNAKNITNIPANISFEVL
MMTGLLPDKVGGVASSLYFSLPKEKISHIIFTSNKQVKSKEDALN
NPYVKVMRRLGIIDESQVIFWDSLKQL,
or a catalytically
active variant thereof.

In some examples, the sialyltransferase is PmST2 (UniProtKB Accession No. Q9CNC4) or a catalytically active variant thereof. PmST2 are variants thereof are described in U.S. Pat. No. 9,102,967, which is incorporated herein by reference in its entirety. In some examples, the sialyltransferase comprises the polypeptide sequence:

(SEQ ID NO: 3)
NLIICCTPLQVLIAEKIIAKFPHTPFYGVMLSTVSNKKFDFYAKR
LAQQCQGFFSMVQHKDRFNLLKEILYLKRTFSGKHFDQVFVANIN
DLQIQFLLSAIDFNLLNTFDDGTINIVPNSLFYQDDPATLQRKLI
NVLLGNKYSIQSLRALSHTHYTIYKGFKNIIERVEPIELVAADNS
EKVTSAVINVLLGQPVFAEDERNIALAERVIKQFNIHYYLPHPRE
KYRLAQVNYIDTELIFEDYILQQCQTHKYCVYTYFSSAIINIMNK
SDNIEVVALKIDTENPAYDACYDLFDELGVNVIDIRE,
or a catalytically
active variant thereof.

In some examples, the sialyltransferase is PmST3 or a catalytically active variant thereof, as described in U.S. Pat. No. 9,783,838, which is incorporated herein by reference in its entirety. In some examples, the sialyltransferase is PmST3Δ35 or a catalytically active variant thereof. In some examples, the sialyltransferase comprises the polypeptide sequence:

(SEQ ID NO: 4)
DKFAEHEIPKAVIVAGNGESLSQIDYRLLPKNYDVFRCNQFYFEE
RYFLGNKIKAVFFTPGVFLEQYYTLYHLKRNNEYFVDNVILSSFN
HPTVDLEKSQKIQALFIDVINGYEKYLSKLTAFDVYLRYKELYEN
QRITSGVYMCAVAIAMGYTDIYLTGIDFYQASEENYAFDNKKPNI
IRLLPDFRKEKTLFSYHSKDIDLEALSFLQQHYHVNFYSISPMSP
LSKHFPIPTVEDDCETTEVAPLKENYINDILLVDKLAAALE,
or a catalytically
active variant thereof.

In some examples, the sialyltransferase is Psp2,6ST or a catalytically active variant thereof, as described in U.S. Patent Publication No. 2016/0177275 (Chen), which is incorporated herein by reference in its entirety. In some examples, the sialyltransferase is Psp26ST (15-501) A366G or a catalytically active variant thereof. In some examples, the sialyltransferase comprises the polypeptide sequence:

(SEQ ID NO: 5)
CNNSEENTQSIIKNDINKTIIDEEYVNLEPINQSNISFTKHSWVQ
TCGTQQLLTEQNKESISLSVVAPRLDDDEKYCFDFNGVSNKGEKY
ITKVTLNVVAPSLEVYVDHASLPTLQQLMDIIKSEEENPTAQRYI
AWGRIVPTDEQMKELNITSFALINNHTPADLVQEIVKQAQTKHRL
NVKLSSNTAHSFDNLVPILKELNSFNNVTVTNIDLYDDGSAEYVN
LYNWRDTLNKTDNLKIGKDYLEDVINGINEDTSNTGTSSVYNWQK
LYPANYHFLRKDYLTLEPSLHELRDYIGDSLKQMQWDGFKKFNSK
QQELFLSIVNFDKQKLQNEYNSSNLPNFVFTGTTVWAGNHEREYY
AKQQINVINNAINESSPHYLGNSYDLFFKGHPGGGIINTLIMQNY
PSMVDIPSKISFEVLMMTDMLPDAVAGIASSLYFTIPAEKIKFIV
FTSTETITDRETALRSPLVQVMIKLGIVKEENVLFWA,
or a catalytically active
variant thereof.

In some examples, the sialyltransferase is Pd2,6ST (GenBank Accession No. BAA25316) or a catalytically active variant thereof. Pd2,6ST and variants thereof are described, for example by Sun and Chen, et al. (Biotechnol Lett (2008) 30:671-676), which is incorporated herein by reference in its entirety. In some examples, the sialyltransferase is Δ15Pd2,6ST (N) or a catalytically active variant thereof. In some examples, the sialyltransferase comprises the polypeptide sequence:

(SEQ ID NO: 6)
CNSDNTSLKETVSSNSADVVETETYQLTPIDAPSSFLSHSWEQTC
GTPILNESDKQAISFDFVAPELKQDEKYCFTFKGITGDHRYITNT
TLTVVAPTLEVYIDHASLPSLQQLIHIIQAKDEYPSNQRFVSWKR
VTVDADNANKLNIHTYPLKGNNTSPEMVAAIDEYAQSKNRLNIEF
YTNTAHVFNNLPPIIQPLYNNEKVKISHISLYDDGSSEYVSLYQW
KDTPNKIETLEGEVSLLANYLAGTSPDAPKGMGNRYNWHKLYDTD
YYFLREDYLDVEANLHDLRDYLGSSAKQMPWDEFAKLSDSQQTLF
LDIVGFDKEQLQQQYSQSPLPNFIFTGTTTWAGGETKEYYAQQQV
NVINNAINETSPYYLGKDYDLFFKGHPAGGVINDIILGSFPDMIN
IPAKISFEVLMMTDMLPDTVAGIASSLYFTIPADKVNFIVFTSSD
TITDREEALKSPLVQVMLTLGIVKEKDVLFWA,
or a catalytically active
variant thereof.

In some examples, the sialyltransferase is CstII (GenBank Accession No. CS299360) or a catalytically active variant thereof. CstII and variants thereof are described by Cheng and Chen, et al. (Glycobiology, 18 (9): 686-697, 2008), which is incorporated herein by reference in its entirety. In some examples, the sialyltransferase is C-His₆-tagged CstII-Δ32^{I535} or a catalytically active variant thereof. In some examples, the sialyltransferase comprises the polypeptide sequence:

(SEQ ID NO: 7)
KKVIIAGNGPSLKEIDYSRLPNDFDVFRCNQFYFEDKYYLGKKCK
AVFYNPSLFFEQYYTLKHLIQNEYETELIMCSNYNQAHLENENFV
KTFYDYFPDAHLGYDFFKQLKDFNAYFKFHEIYFNQRITSGVYMC
AVAIALGYKEIYLSGIDFYQNGSSYAFDTKQKNLLKLAPNFKNDN
SHYIGHSKNTDIKALEFLEKTYKIKLYCLCPNSLLANFIELAPNL
MSNFIIQEKNNYTKDILIPSSEAYGKFSKNIN,
or a catalytically
active variant thereof.

Sialidases can also be use in methods for forming sialic acids, as described above. In some examples, the sialidase is SpNanB (NCBI Accession No. NP_359124.1) or a catalytically active variant thereof. In some examples, the sialidase comprises the polypeptide sequence:

(SEQ ID NO: 8)
NELNYGQLSISPIFQGGSYQLNNKSIDISPLLLDKLSGDSQTVVM
KFKADKPNSLQALFGLSNSKAGFKNNYFSIFMRDSGEIGVEIRDA
QKGINYLFSRPASLWGKHKGQAVENTLVFVSDSKDKTYTMYVNGI
EVFSETVDTFLPISNINGIDKATLGAVNREGKEHYLAKGSIDEIS
LENKAISDQEVSTIPLSNPFQLIFQSGDSTQANYFRIPTLYTLSS
GRVLSSIDARYGGTHDSKSKINIATSYSDDNGKTWSEPIFAMKFN
DYEEQLVYWPRDNKLKNSQISGSASFIDSSIVEDKKSGKTILLAD
VMPAGIGNNNANKADSGFKEINGHYYLKLKKNGDNDFRYTVRENG
VVYDETTNKPTNYTINDKYEVLEGGKSLTVEQYSVDFDSGSLRER
HNGKQVPMNVFYKDSLFKVTPTNYIAMTTSQNRGESWEQFKLLPP
FLGEKHNGTYLCPGQGLALKSSNRLIFATYTSGELTYLISDDSGQ
TWKKSSASIPFENATAEAQMVELRDGVIRTFFRTTTGKIAYMTSR
DSGETWSEVSYIDGIQQTSYGTQVSAIKYSQLIDGKEAVILSTPN
SRSGRKGGQLVVGLVNKEDDSIDWKYHYDIDLPSYGYAYSAITEL
PNHHIGVLFEKYDSWSRNELHLSNVVQYIDLEINDLTK,
or a catalytically active
variant thereof.

In some examples, the sialidase is SpNanC (NCBI Accession No. WP_024478413.1) or a catalytically active variant thereof. In some examples, the sialidase comprises the polypeptide sequence:

(SEQ ID NO: 9)
KKNIKQYVTLGTVVVLSAFVANSVAAQETETSEVSTPELVQPVAP
TTSISEVQHKSGNSSEVTVQPRTVETTVKDPSSTAEETLVLEKNN
VTLTGGGENVTKELKDKFTSGDFTVVIKYNQSSEKGLQALFGISN
SKPGQQNSYVDVFLRDNGELGMEARDTSSNKNNLVSRPASVWGKY
KQEAVTNTVAVVADSVKKTYSLYANGTKVVEKKVDNFLNIKDIKG
IDYYMLGGVKRAGKTAFGFNGTLENIKFFNSALDEETVKKMTTNA
VTGHLIYTANDTTGSNYFRIPVLYTFSNGRVFSSIDARYGGTHDF
LNKINIATSYSDDNGKTWTKPKLTLAFDDFAPVPLEWPREVGGRD
LQISGGATYIDSVIVEKKNKQVLMFADVMPAGVSFREATRKDSGY
KQIDGNYYLKLRKQGDTDYNYTIRENGTVYDDRTNRPTEFSVDKN
FGIKQNGNYLTVEQYSVSFENNKKTEYRNGTKVHMNIFYKDALFK
VVPTNYIAYISSNDHGESWSAPTLLPPIMGLNRNAPYLGPGRGII
ESSTGRILIPSYTGKESAFIYSDDNGASWKVKVVPLPSSWSAEAQ
FVELSPGVIQAYMRTNNGKIAYLTSKDAGTTWSAPEYLKFVSNPS
YGTQLSIINYSQLIDGKKAVILSTPNSTNGRKHGQIWIGLINDDN
TIDWRYHHDVDYSNYGYSYSTLTELPNHEIGLMFEKFDSWSRNEL
HMKNVVPYITFKIEDLKKN,
or a catalytically active variant
thereof.

In some examples, the sialidase is S. pneumoniae TIGR4 SpNanC (NCBI Accession No. AAK75424.1) or a catalytically active variant thereof. In some examples, the sialidase comprises the polypeptide sequence:

(SEQ ID NO: 10)
KKNIKQYVTLGTVVVLSAFVANSVAAQETETSEVSTPKLVQPVAP
TTPISEVQPTSDNSSEVTVQPRTVETTVKDPSSTAEETPVLEKNN
VTLTGGGENVTKELKDKFTSGDFTVVIKYNQSSEKGLQALFGISN
SKPGQQNSYVDVFLRDNGELGMEARDTSSNKNNLVSRPASVWGKY
KQEAVTNTVAVVADSVKKTYSLYANGTKVVEKKVDNFLNIKDIKG
IDYYMLGGVKRAGKTAFGFNGTLENIKFFNSALDEETVKKMTTNA
VTGHLIYTANDTTGSNYFRIPVLYTFSNGRVFSSIDARYGGTHDF
LNKINIATSYSDDNGKTWTKPKLTLAFDDFAPVPLEWPREVGGRD
LQISGGATYIDSVIVEKKNKQVLMFADVMPAGVSFREATRKDSGY
KQIDGNYYLKLRKQGDTDYNYTIRENGTVYDDRTNRPTEFSVDKN
FGIKQNGNYLTVEQYSVSFENNKKTEYRNGTKVHMNIFYKDALFK
VVPTNYIAYISSNDHGESWSAPTLLPPIMGLNRNAPYLGPGRGII
ESSTGRILIPSYTGKESAFIYSDDNGASWKVKVVPLPSSWSAEAQ
FVELSPGVIQAYMRTNNGKIAYLTSKDAGTTWSAPEYLKFVSNPS
YGTQLSIINYSQLIDGKKAVILSTPNSTNGRKHGQIWIGLINDDN
TIDWRYHHDVDYSNYGYSYSTLTELPNHEIGLMFEKFDSWSRNEL
HMKNVVPYITFKIEDLKKN.

Methods for preparing sialosides described herein generally include providing reaction mixtures that contain the one or more enzymes as described herein, e.g., a CMP-sialic acid synthetase (CSS) or a sialyltransferase (ST). A CSS, or an ST can be, for example, isolated or otherwise purified prior to addition to the reaction mixture. As used herein, a “purified” enzyme refers to an enzyme which is provided as a purified protein composition wherein the enzyme constitutes at least about 50% of the total protein in the purified protein composition. For example, the enzyme can constitute about 50%, 60%, 70%, 75%, 80%, 85%, 90%, 95%, or 99% of the total protein in the purified protein composition. In some examples, the enzymes in the reaction mixture are provided as purified protein compositions wherein the enzyme constitutes at least about 95% of the total protein in purified protein composition (prior to addition to the reaction mixture). The amount of the enzyme in a purified protein composition can be determined by any number of known methods including, for example, by polyacrylamide gel electrophoresis (e.g., SDS-PAGE) followed by detection with a staining reagent (e.g., Coomassie Brilliant Blue G-250, a silver nitrate stain, and/or a reagent containing a Psp26ST antibody). The enzymes used in the methods for forming sialosides can also be secreted by a cell present in the reaction mixture. Alternatively, the enzymes can catalyze the reaction within a cell expressing the variant.

Reaction mixtures can contain additional reagents for use in glycosylation techniques. For example, in certain examples, the reaction mixtures can contain buffers (e.g., 2-(N-morpholino) ethanesulfonic acid (MES), 2-[4-(2-hydroxyethyl) piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂), and salts of Mn²⁺ and Mg²⁺), detergents/surfactants (e.g., a non-ionic surfactant such as N,N-bis[3-(D-gluconamido) propyl]cholamide, polyoxyethylene (20) cetyl ether, dimethyldecylphosphine oxide, branched octylphenoxy poly(ethyleneoxy) ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl (tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[bis(carboxymethyl)-amino]ethyl} (carboxymethyl)amino) acetic acid (EDTA), and 1,2-bis(o-aminophenoxy) ethane-N,N,N′,N′-tetraacetic acid (BAPTA)), reducing agents (e.g., dithiothreitol (DTT), β-mercaptoethanol (BME), and tris(2-carboxyethyl)phosphine (TCEP)), and labels (e.g., fluorophores, radiolabels, and spin labels). Buffers, cosolvents, salts, detergents/surfactants, chelators, reducing agents, and labels can be used at any suitable concentration, which can be readily determined by one of skill in the art. In general, buffers, cosolvents, salts, detergents/surfactants, chelators, reducing agents, and labels are included in reaction mixtures at concentrations ranging from about 1 μM to about 1 M. For example, a buffer, a cosolvent, a salt, a detergent/surfactant, a chelator, a reducing agent, or a label can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some examples, the reaction mixture can contain CTP, a CSS, an ST, a compound as described herein, and one or more components selected from a buffer, a cosolvent, a salt, a detergent/surfactant, a chelator, a reducing agent, and a label.

The enzymatic reactions can be conducted at any suitable temperature. In general, the reactions are conducted at a temperature of from about 4° C. to about 50° C. The reactions can be conducted, for example, at about 25° C., about 30° C., or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods described herein, depending on the identity of a particular enzymes, sialic acids, or acceptor molecules employed.

### V. Sialosides Containing a 8-N-Derivatized Sialic Acid

The compounds described herein can be useful as substrates and/or inhibitors to neuraminidases. In some examples, the compounds inhibit influenza A virus neuraminidases and/or influenza B virus neuraminidases. In some examples, the compounds selectively inhibit influenza A virus neuraminidases. In some examples, the compounds selectively inhibit influenza B virus neuraminidases.

Accordingly, provided herein are compounds according to Formula III,

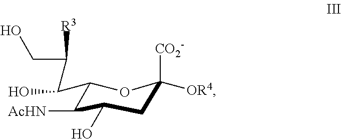

or a pharmaceutically acceptable salt thereof, wherein:

R³ is selected from the group consisting of NHAc, NH₂, and N₃; and R⁴ is selected from the group consisting of H, C₁-C₆ alkyl, C₂-C₆ alkenyl, C₂-C₆ alkynyl, 4-methylumbelliferone (MU), para-nitrophenyl (pNP), indole, a monosaccharide, an oligosaccharide, a glycolipid, a glycopeptide, a glycoprotein, a galactoside, GalβMU, GalβpNP, or Galβ1-4GlcNAcβOR. In some examples, R³ is NH₂. In some examples, R³ is N₃.

### VI. Chemoenzymatic Synthesis of Sialosides Containing 8-N-Derivatized Sialic Acid

Any suitable sialic acid aldolase (i.e., NCBI Accession No. WP_005723432 or a catalytically active variant thereof) can be used in the methods for forming the sialosides disclosed herein. Sialic acid aldolase refers to an aldolase that catalyzes a reversible reaction that converts a suitable hexosamine, hexose, pentose, or derivative (such as N-acetyl mannosamine) to sialic acid via reaction with pyruvate. For example, sialic acid aldolase from Pasteurella multocida (PmAldolase), E. coli can be used. In some examples, the sialic acid aldolase is Pasteurella multocida sialic acid aldolase (PmAldolase).

In some examples, the sialic acid aldolase is PmAldolase. In some examples, the sialic acid aldolase comprises the polypeptide sequence, SEQ ID NO: 11:

TNIAIIPARAGSKGIPDKNLQPVGGHSLIGRAILAAKNADVFDMI
VVTSDGDNILREAEKYGALALKRPAELAQDNSRTIDAILHALESL
NIREGTCTLLQPTSPLRDHLDIKNAMDMYVNGGVHSVVSACECEH
HPYKAFALSKDHEVLPVREIADFEAVRQTLPKMYRANGAIYINDI
AQLLKEKYFFIPPLKFYLMPTYHSVDIDVKQDLELAEILSNK,
or a catalytically active variant thereof.

Any suitable CMP-sialic acid synthetase (i.e., N-acylneuraminate cytidylyltransferase, EC 2.7.7.43, also referred to as “CSS”) can be used in the methods for forming the sialosides disclosed herein. For example, CMP-sialic acid synthetases from E. coli, C. thermocellum, S. agalactiae, or N. meningitidis can be used. In some examples, the CMP sialic acid synthetase is Neisseria meningitidis CMP-sialic acid synthetase (NmCSS).

In some examples, the CMP-sialic acid synthetase is NmCSS (NCBI Accession No. WP_025459740.1) or a catalytically active variant thereof. In some examples, the CMP-sialic acid synthetase comprises the polypeptide sequence SEQ ID NO: 1, or a catalytically active variant thereof.

Any suitable sialyltransferase (also referred to as “ST”) can be used in the methods for forming the sialosides disclosed herein. In some examples, the sialyltransferase is a beta-galactoside alpha-2,3-sialyltransferases belonging to Glycosyltransferase family 80 (GT80 using CAZy nomenclature), which catalyzes the following conversion: CMP-sialic acid+β-D-galactosyl-R=CMP+α-sialic acid-(2→3)-β-D-galactosyl-R, where the acceptor is GalβOR, where R is H, a monosaccharide, an oligosaccharide, a polysaccharide, a glycopeptide, a glycoprotein, a glycolipid, or a hydroxyl-containing compound. GT80 family sialyltransferases also include galactoside or N-acetylgalactosaminide alpha-2,6-sialyltransferases that catalyze the following conversion: CMP-sialic acid+galactosyl/GalNAc-R=CMP+α-sialic acid-(2→6)-β-D-galactosyl/GalNAc-R, where the acceptor is GalOR or GalNAcOR, where R is H, serine or threonine on a peptide or protein, a monosaccharide, an oligosaccharide, a polysaccharide, a glycopeptide, a glycoprotein, a glycolipid, or a hydroxyl-containing compound.

Sialyltransferases in family EC 2.4.99, such as beta-galactosaminide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8), and lactosylceramide alpha-2,3-sialyltransferase (EC 2.4.99.9), can also be used in the methods for preparing sialosides.

In some examples, the sialyltransferase is selected from the group consisting of PmST1, a PmST1 variant, PmST2, a PmST2 variant, PmST3, a PmST3 variant, Pd2,6ST, Psp2,6ST, CstI, CstII, and a polysialyltransferase.

In some examples, the sialyltransferase is PmST1 (NCBI Accession No. WP_005753497.1) or a catalytically active variant thereof. In some examples, the sialyltransferase is PmST1_M144D or a catalytically active variant thereof. In some examples, the sialyltransferase comprises the polypeptide sequence SEQ ID NO: 2, or a catalytically active variant thereof.

In some examples, the sialyltransferase is PmST2 (UniProtKB Accession No. Q9CNC4) or a catalytically active variant thereof. PmST2 are variants thereof are described in U.S. Pat. No. 9,102,967, which is incorporated herein by reference in its entirety. In some examples, the sialyltransferase comprises the polypeptide sequence SEQ ID NO: 3, or a catalytically active variant thereof.

In some examples, the sialyltransferase is PmST3 or a catalytically active variant thereof, as described in U.S. Pat. No. 9,783,838, which is incorporated herein by reference in its entirety. In some examples, the sialyltransferase is PmST3435 or a catalytically active variant thereof. In some examples, the sialyltransferase comprises the polypeptide sequence SEQ ID NO: 4, or a catalytically active variant thereof.

In some examples, the sialyltransferase is Psp2,6ST or a catalytically active variant thereof, as described in U.S. Pat. Appl. Pub. No. 2016/0177275, which is incorporated herein by reference in its entirety. In some examples, the sialyltransferase is Psp26ST (15-501) A366G or a catalytically active variant thereof. In some examples, the sialyltransferase comprises the polypeptide sequence SEQ ID NO: 5, or a catalytically active variant thereof.

In some examples, the sialyltransferase is Pd2,6ST (GenBank Accession No. BAA25316) or a catalytically active variant thereof. Pd2,6ST are variants thereof are described, for example by Sun and Chen, et al. (Biotechnol Lett (2008) 30:671-676), which is incorporated herein by reference in its entirety. In some examples, the sialyltransferase is Δ15Pd2,6ST (N) or a catalytically active variant thereof. In some examples, the sialyltransferase comprises the polypeptide sequence SEQ ID NO: 6, or a catalytically active variant thereof.

In some examples, the sialyltransferase is CstII (GenBank Accession No. CS299360) or a catalytically active variant thereof. CstII are variants thereof are described by Cheng and Chen, et al. (Glycobiology, 18 (9): 686-697, 2008), which is incorporated herein by reference in its entirety. In some examples, the sialyltransferase is C-His₆-tagged CstII-Δ32^{I53S} or a catalytically active variant thereof. In some examples, the sialyltransferase comprises the polypeptide sequence SEQ ID NO: 7, or a catalytically active variant thereof.

Sialidases can also be used in methods for forming sialic acids, as described above. In some examples, the sialidase is SpNanB (NCBI Accession No. NP_359124.1) or a catalytically active variant thereof. In some examples, the sialidase comprises the polypeptide sequence SEQ ID NO: 8, or a catalytically active variant thereof.

In some examples, the sialidase is SpNanC (NCBI Accession No. WP_024478413.1) or a catalytically active variant thereof. In some examples, the sialidase comprises the polypeptide sequence SEQ ID NO: 9, or a catalytically active variant thereof.

In some examples, the sialidase is S. pneumoniae TIGR4 SpNanC (NCBI Accession No. AAK75424.1) or a catalytically active variant thereof. In some examples, the sialidase comprises the polypeptide sequence SEQ ID NO: 10.

Methods for preparing sialosides described herein generally include providing reaction mixtures that contain the one or more enzymes as described herein, e.g., a CMP-sialic acid synthetase (CSS) or a sialyltransferase (ST). A CSS, or an ST can be, for example, isolated or otherwise purified prior to addition to the reaction mixture. As used herein, a “purified” enzyme refers to an enzyme which is provided as a purified protein composition wherein the enzyme constitutes at least about 50% of the total protein in the purified protein composition. For example, the enzyme can constitute about 50%, 60%, 70%, 75%, 80%, 85%, 90%, 95%, or 99% of the total protein in the purified protein composition. In some examples, the enzymes in the reaction mixture are provided as purified protein compositions wherein the enzyme constitutes at least about 95% of the total protein in purified protein composition (prior to addition to the reaction mixture). The amount of the enzyme in a purified protein composition can be determined by any number of known methods including, for example, by polyacrylamide gel electrophoresis (e.g., SDS-PAGE) followed by detection with a staining reagent (e.g., Coomassie Brilliant Blue G-250, a silver nitrate stain, and/or a reagent containing a Psp26ST antibody). The enzymes used in the methods for forming sialosides can also be secreted by a cell present in the reaction mixture. Alternatively, the enzymes can catalyze the reaction within a cell expressing the variant.

Reaction mixtures can contain additional reagents for use in glycosylation techniques. For example, in certain examples, the reaction mixtures can contain buffers (e.g., 2-(N-morpholino) ethanesulfonic acid (MES), 2-[4-(2-hydroxyethyl) piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂), and salts of Mn²⁺ and Mg²⁺), detergents/surfactants (e.g., a non-ionic surfactant such as N,N-bis[3-(D-gluconamido) propyl]cholamide, polyoxyethylene (20) cetyl ether, dimethyldecylphosphine oxide, branched octylphenoxy poly(ethyleneoxy) ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl (tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[bis(carboxymethyl)amino]-ethyl} (carboxymethyl)amino) acetic acid (EDTA), and 1,2-bis(o-aminophenoxy) ethane-N,N,N′,N′-tetraacetic acid (BAPTA)), reducing agents (e.g., dithiothreitol (DTT), β-mercaptoethanol (BME), and tris(2-carboxyethyl)phosphine (TCEP)), and labels (e.g., fluorophores, radiolabels, and spin labels). Buffers, cosolvents, salts, detergents/surfactants, chelators, reducing agents, and labels can be used at any suitable concentration, which can be readily determined by one of skill in the art. In general, buffers, cosolvents, salts, detergents/surfactants, chelators, reducing agents, and labels are included in reaction mixtures at concentrations ranging from about 1 μM to about 1 M. For example, a buffer, a cosolvent, a salt, a detergent/surfactant, a chelator, a reducing agent, or a label can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some examples, the reaction mixture can contain CTP, a CSS, an ST, a compound as described herein, and one or more components selected from a buffer, a cosolvent, a salt, a detergent/surfactant, a chelator, a reducing agent, and a label.

The enzymatic reactions can be conducted at any suitable temperature. In general, the reactions are conducted at a temperature of from about 4° C. to about 50° C. The reactions can be conducted, for example, at about 30° C., 25° C., or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods described herein, depending on the identity of a particular enzymes, sialic acids, or acceptor molecules employed.

### VII. Methods for Inhibiting Neuraminidases

Also provided herein is a method of inhibiting a neuraminidase, including contacting the neuraminidase with a compound of Formula I:

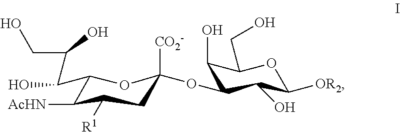

or a pharmaceutically acceptable salt thereof, wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, and ProNH-PEG4-biotin. In some examples, R¹ is NH₂. In some examples, R¹ is NH₂ or guanidine. In some examples, R¹ is N₃.

In some examples, the compound, or pharmaceutically acceptable salt thereof, for use in the methods described herein is selected from the group consisting of:

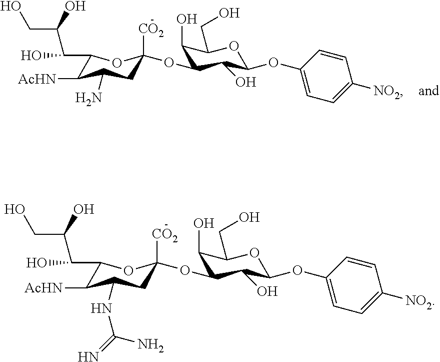

Also provided herein is a method of inhibiting a neuraminidase, including contacting the neuraminidase with a compound of Formula II:

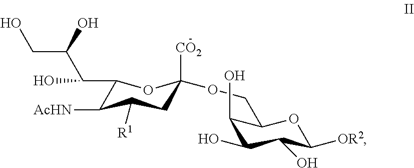

or a pharmaceutically acceptable salt thereof, wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, ProNH-Biotin, and ProNH-PEG4-biotin. In some examples, R¹ is NH₂ or guanidine.

In some examples, the compound for use in the methods described herein includes a compound selected from the group consisting of:

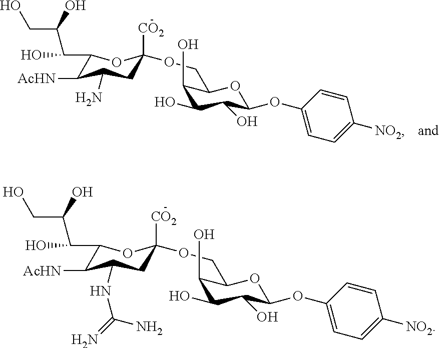

Also provided herein is a method of inhibiting a neuraminidase, including contacting the neuraminidase with a biotinylated compound, or pharmaceutically acceptable salt thereof, selected from the group consisting of

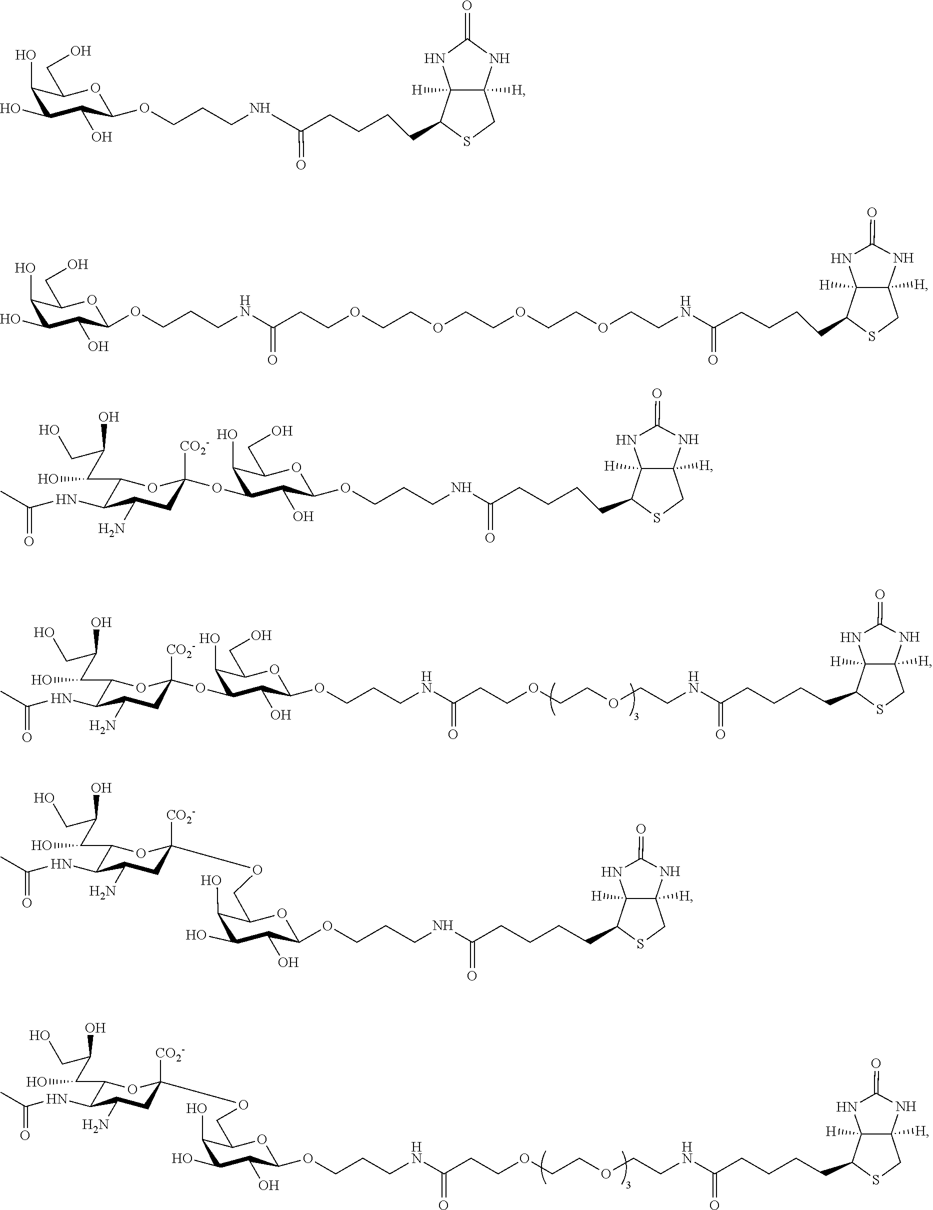

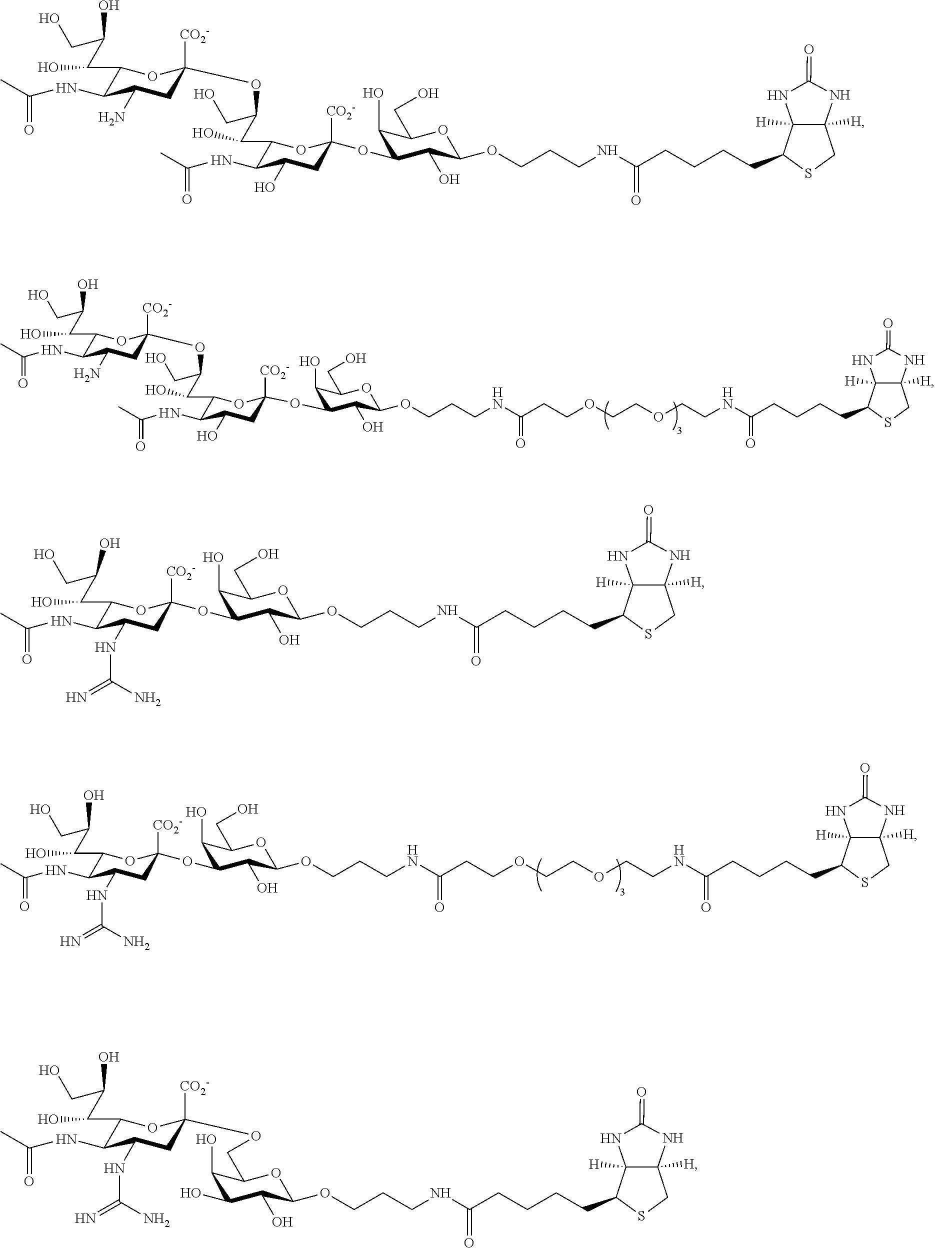

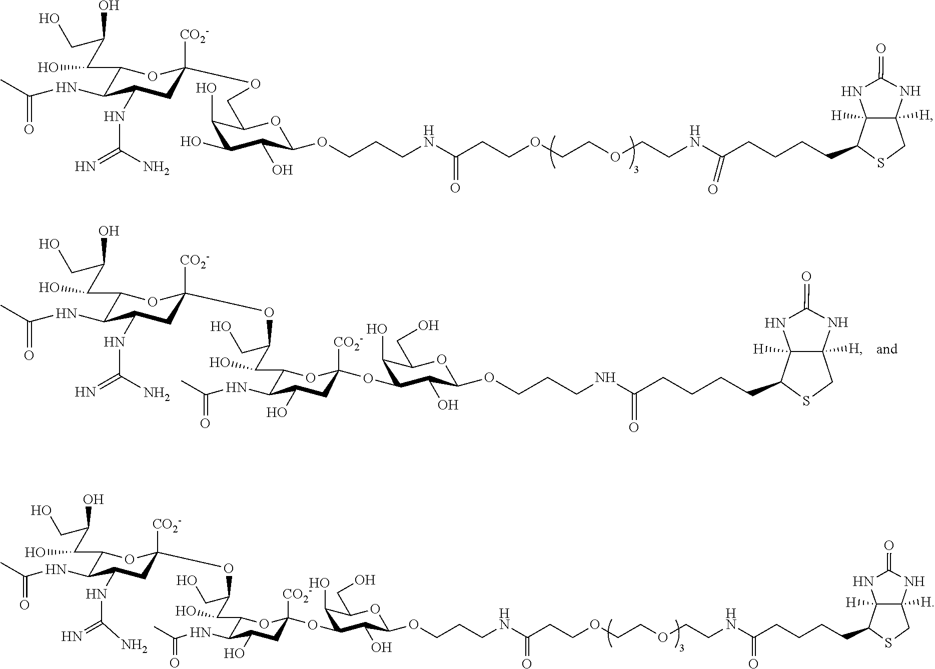

Also provided herein is a method of inhibiting a neuraminidase, including contacting the neuraminidase with an α2-3-linked 4NH₂Neu5Ac-sialoside, or a pharmaceutically acceptable salt thereof, selected from the group consisting of

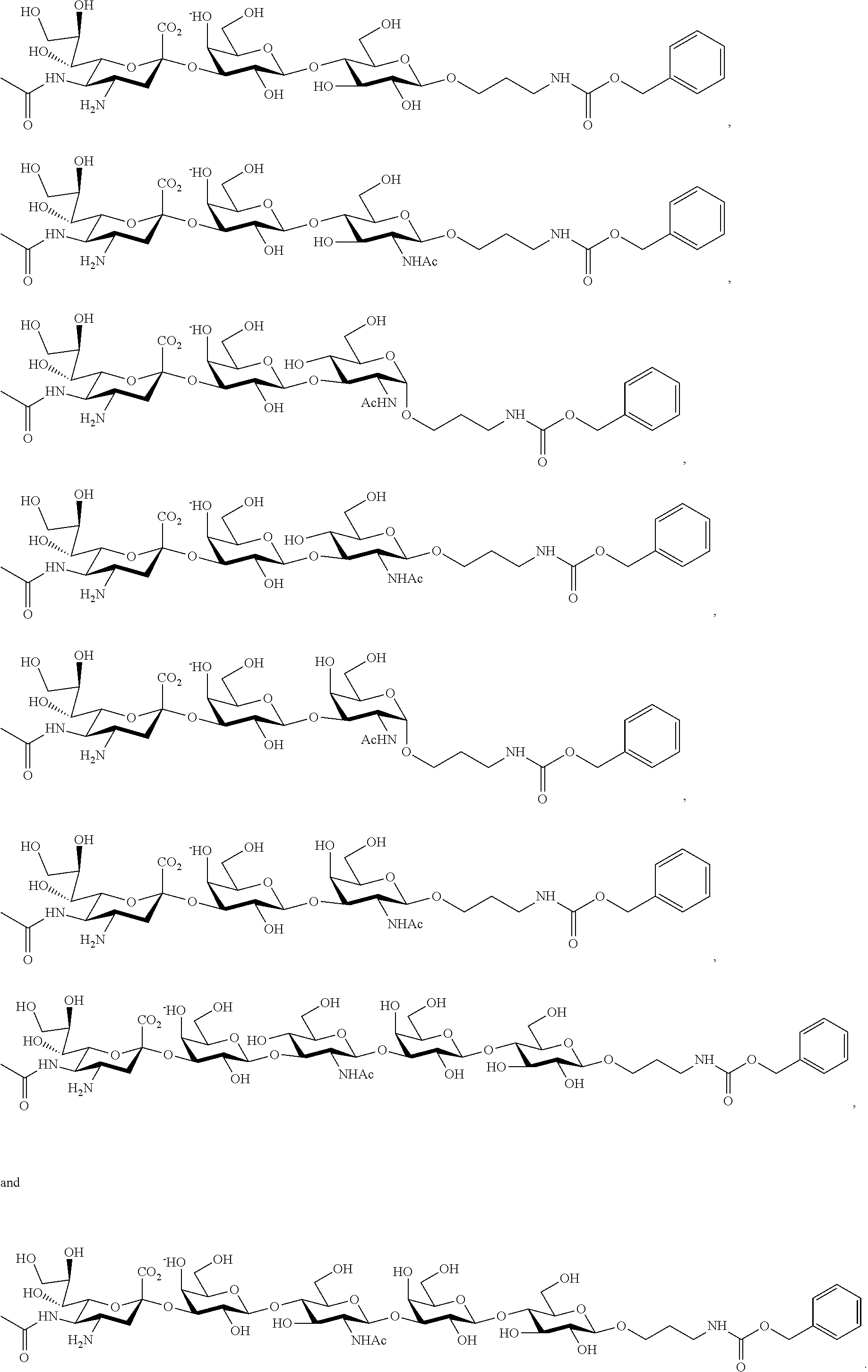

Also provided herein is a method of inhibiting a neuraminidase, including contacting the neuraminidase with an α2-3-linked 4NAcNeu5Ac-sialoside, or a pharmaceutically acceptable salt thereof, selected from the group consisting of

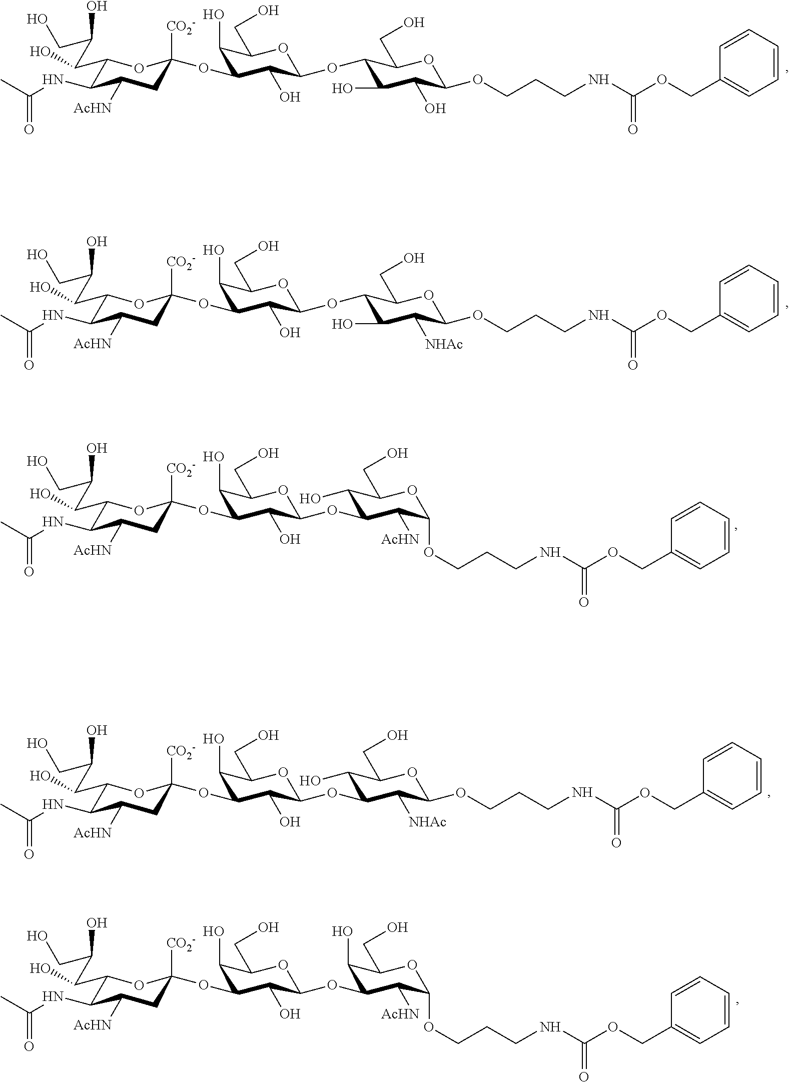

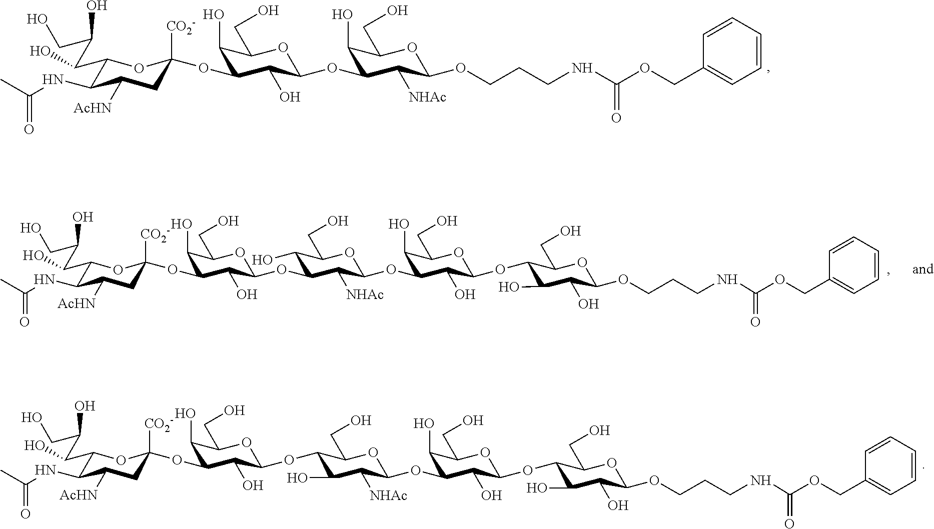

Also provided herein is a method of inhibiting a neuraminidase, including contacting the neuraminidase with an α2-6-linked 4NH₂Neu5Ac-sialoside, or a pharmaceutically acceptable salt thereof, selected from the group consisting of

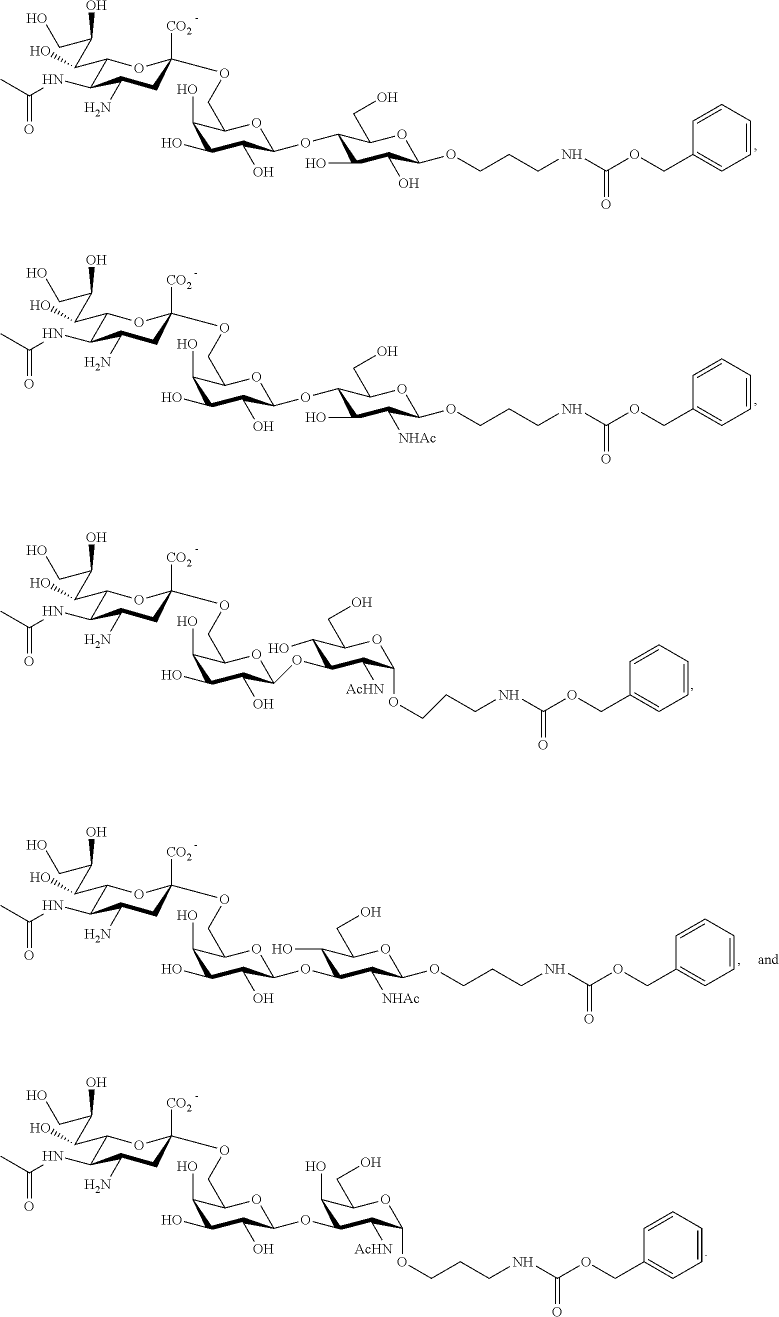

Also provided herein is a method of inhibiting a neuraminidase, including contacting the neuraminidase with an α2-6-linked 4NAcNeu5Ac-sialoside, or a pharmaceutically acceptable salt thereof, selected from the group consisting of

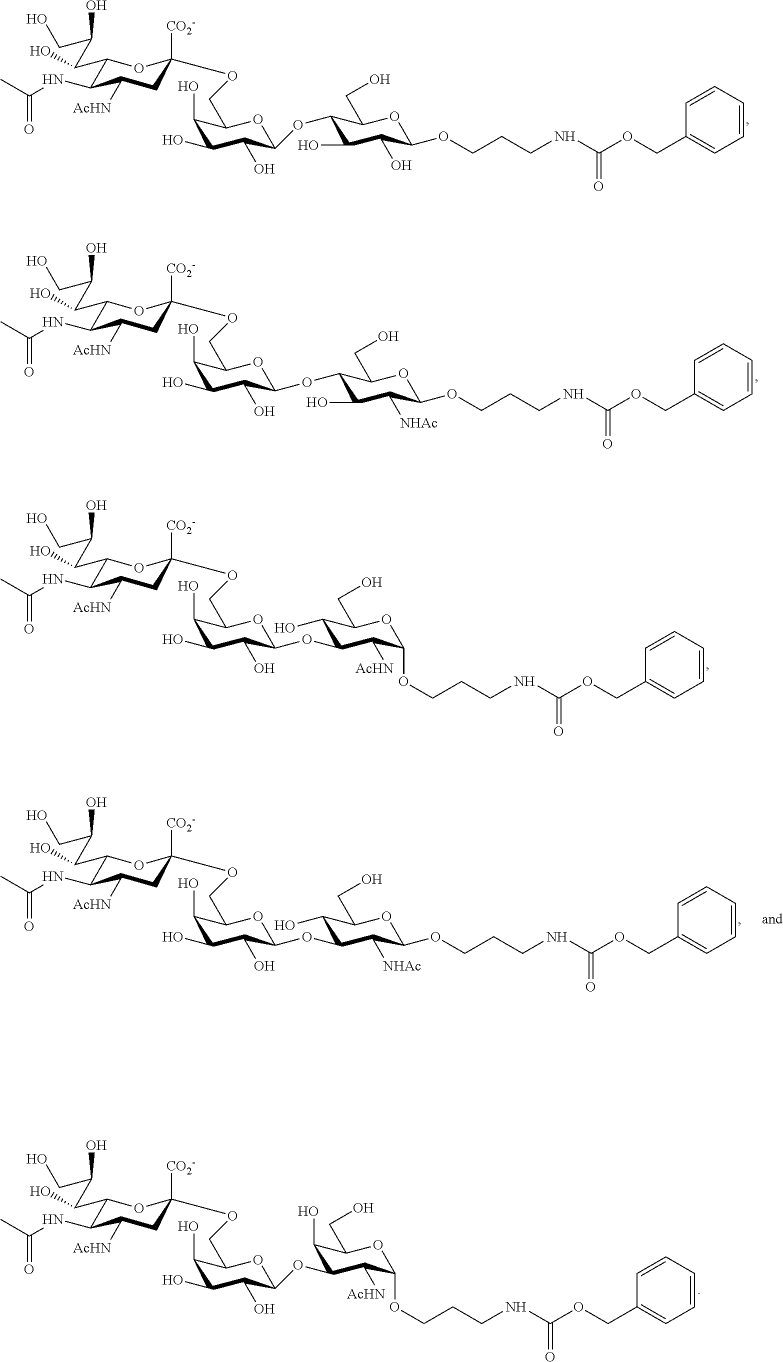

Also provided herein is a method of inhibiting a neuraminidase, including contacting the neuraminidase with 4-methylumbelliferyl (MU)-tagged sialoside, or a pharmaceutically acceptable salt thereof, selected from the group consisting of

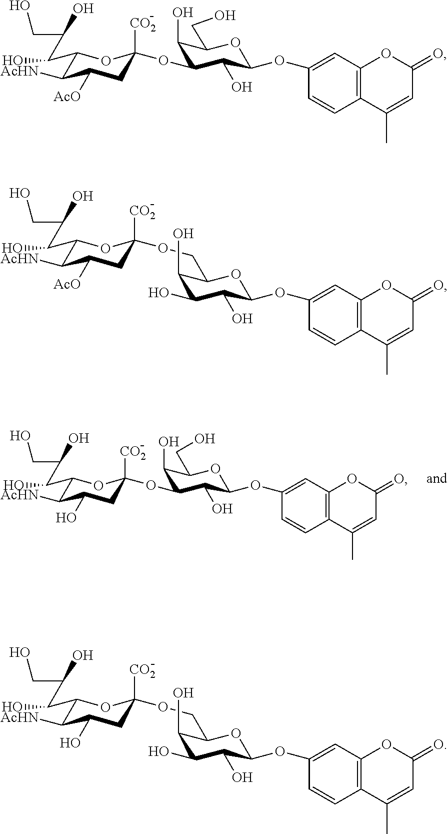

Also provided herein is a method of inhibiting a neuraminidase, including contacting a neuraminidase with a compound of Formula III,

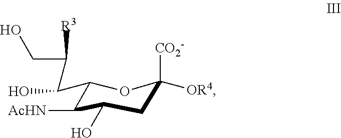

or a pharmaceutically acceptable salt thereof, wherein R³ is NH₂, NHAc, or N₃, and wherein R⁴ is H, C₁-C₆ alkyl, C₂-C₆ alkenyl, C₂-C₆ alkynyl, 4-methylumbelliferone (MU), para-nitrophenyl (pNP), indole, a monosaccharide, an oligosaccharide, a glycolipid, a glycopeptide, a glycoprotein, a galactoside, GalβMU, GalβpNP, or Galβ1-4GlcNAcβOR. In some examples, R³ is NH₂.

The compounds of the methods described herein can inhibit neuraminidases including, but not limited to, human neuraminidases, bacteria neuraminidases, and influenza virus neuraminidases. In some examples, the compounds of the methods described herein have inhibitory activities from at least 30 percent to greater than 90 percent for one or more neuraminidases (e.g., at least 30 percent, at least 40 percent, at least 50 percent, at least 60 percent, at least 70 percent, at least 80 percent, at least 90 percent, or greater than 90 percent). In some examples, the compounds of the methods described herein inhibit influenza A virus neuraminidases and/or influenza B virus neuraminidases. In some examples, the compounds of the methods described herein selectively inhibit influenza A virus neuraminidases and/or influenza B neuraminidases (e.g., the compounds have inhibitory activity for the neuraminidase from at least 50 percent to greater than 90 percent), but do not selectively inhibit human or bacteria neuraminidases (e.g., the compounds have inhibitory activities for the neuraminidase from less than 50 percent, less than 40 percent, less than 30 percent, less than 20 percent, less than 10 percent, less than 5 percent, or less than 1 percent). In some examples, the compounds of the methods described herein selectively inhibit influenza A virus neuraminidases. In some examples, compounds of the methods described herein selectively inhibit influenza B virus neuraminidases.

### VIII. Kits for Determining the Presence of Neuraminidase

Also described herein is a kit for determining the presence of neuraminidase, including a compound, or pharmaceutically acceptable salts thereof, according to Formula I:

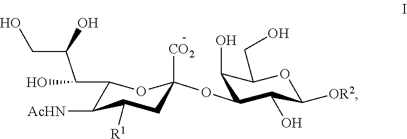

wherein R¹ is NH₂, NHAc, guanidine, or N₃, and wherein R² is 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, or ProNH-PEG4-biotin. In some examples, R¹ is N₃.

In some examples, the compound of the kit described herein is

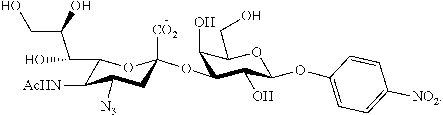

In some examples, the compound of the kit described herein is

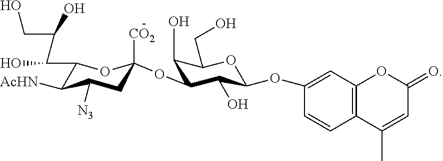

Also provided herein is a kit for determining the presence of neuraminidase, including a compound, or a pharmaceutically acceptable salt thereof, according to Formula II:

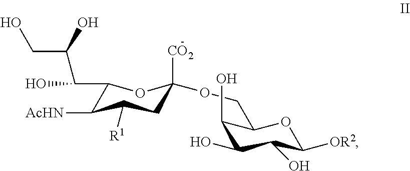

wherein R¹ is NH₂, NHAc, guanidine, or N₃, and wherein R² is 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GalNAcβProNHCbz, ProNH-Biotin, or ProNH-PEG4-biotin. In some examples, R¹ is N₃.

In some examples, the compound of the kit described herein is

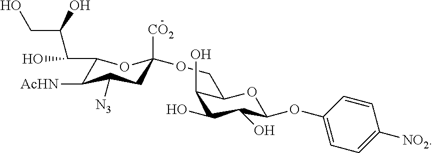

In some examples, the compound of the kit described herein is

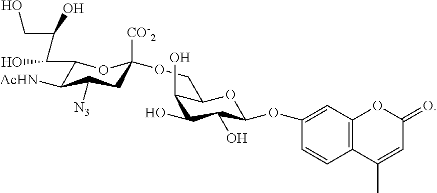

Also provided herein is a kit for determining the presence of neuraminidase, including a biotinylated compound, or pharmaceutically acceptable salt thereof, selected from the group consisting of

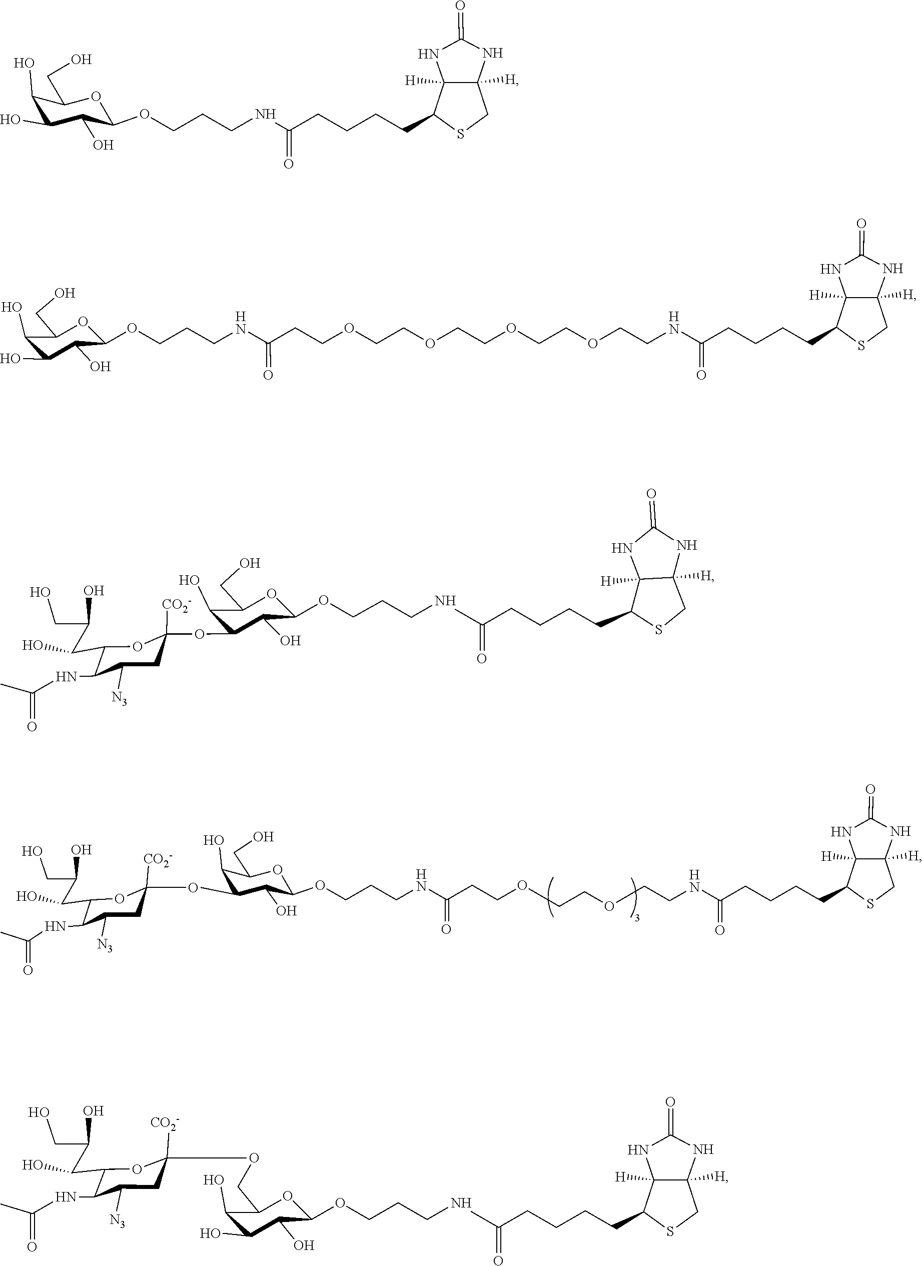

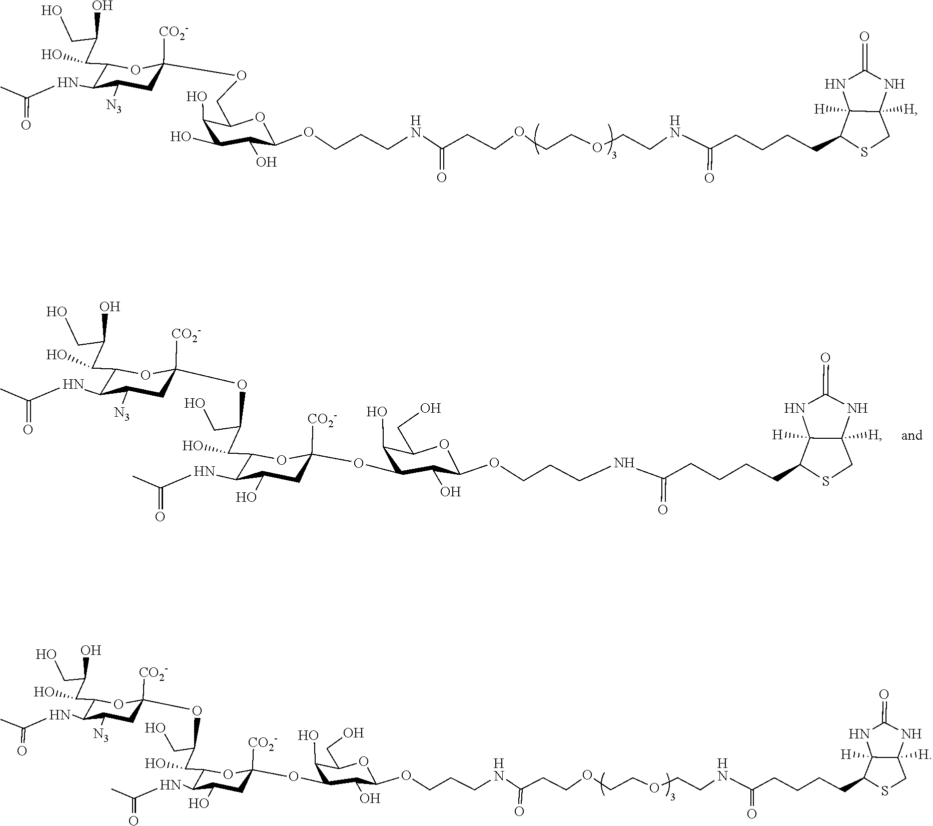

Also provided herein is a kit for determining the presence of neuraminidase, including an α2-3-linked 4NH₂Neu5Ac-sialoside, or pharmaceutically acceptable salt thereof, selected from the group consisting of

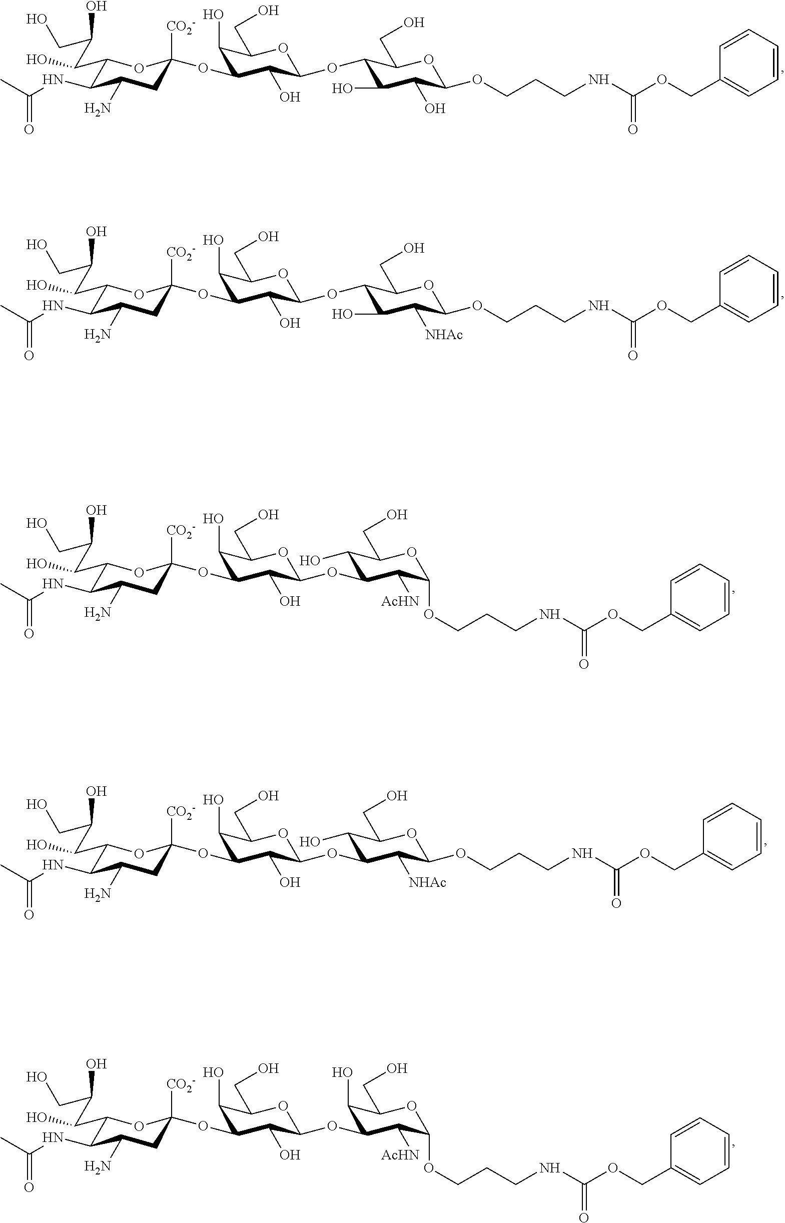

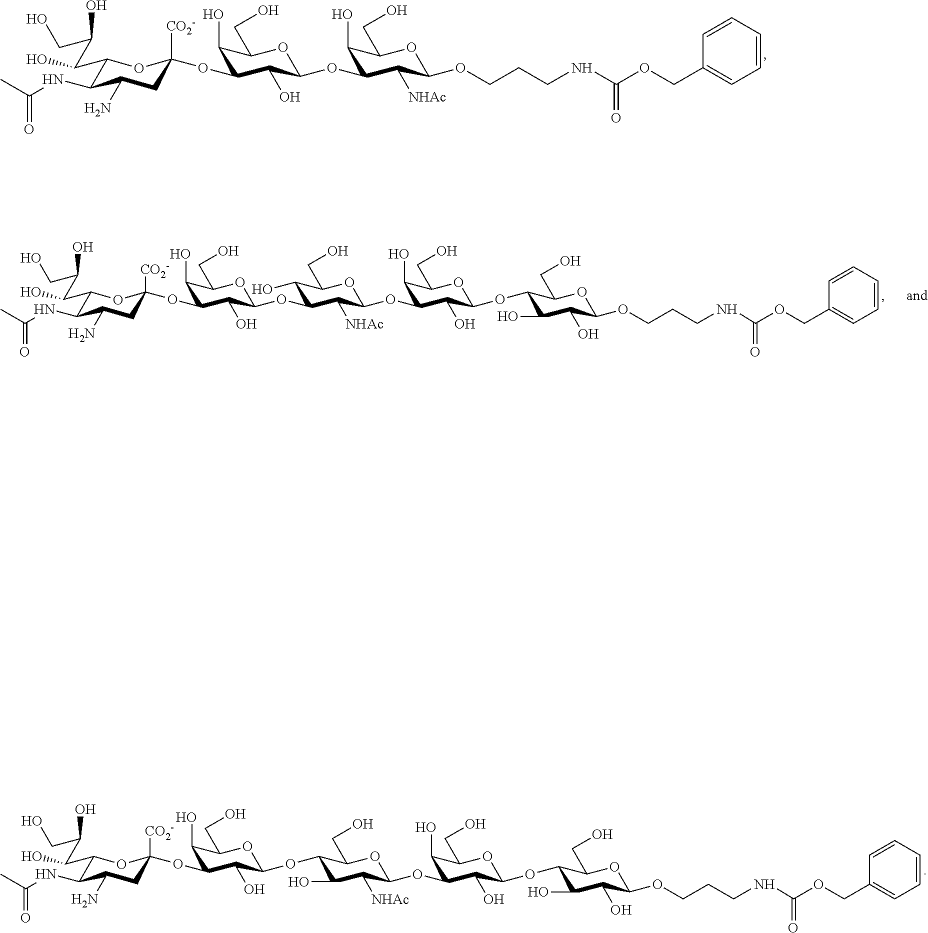

Also provided herein is a kit for determining the presence of neuraminidase, including an α2-3-linked 4NAcNeu5Ac-sialoside, or pharmaceutically acceptable salt thereof, selected from the 5 group consisting of

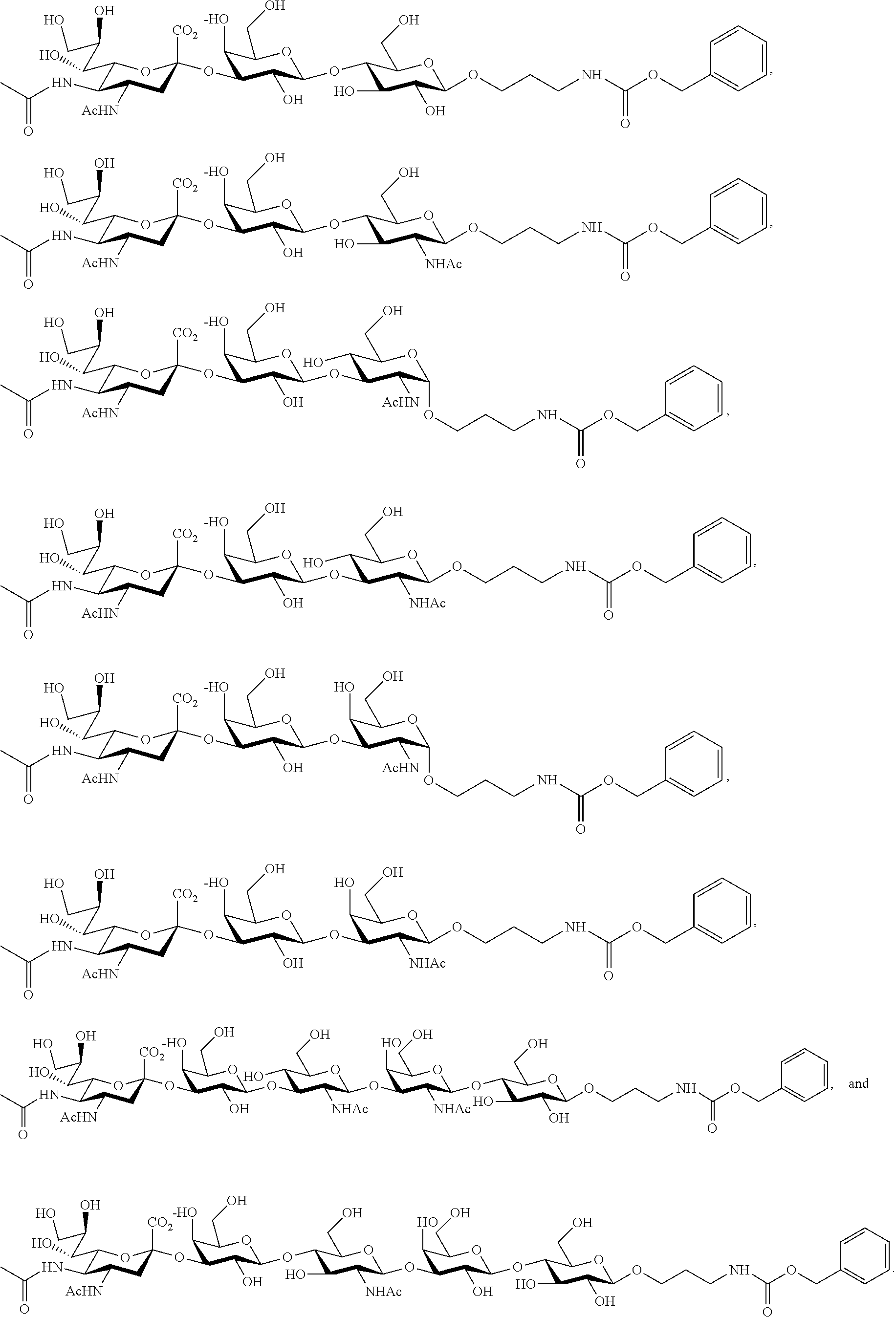

Also provided herein is a kit for determining the presence of neuraminidase, including an α2-6-linked 4NH₂Neu5Ac-sialoside, or pharmaceutically acceptable salt thereof, selected from the group consisting of

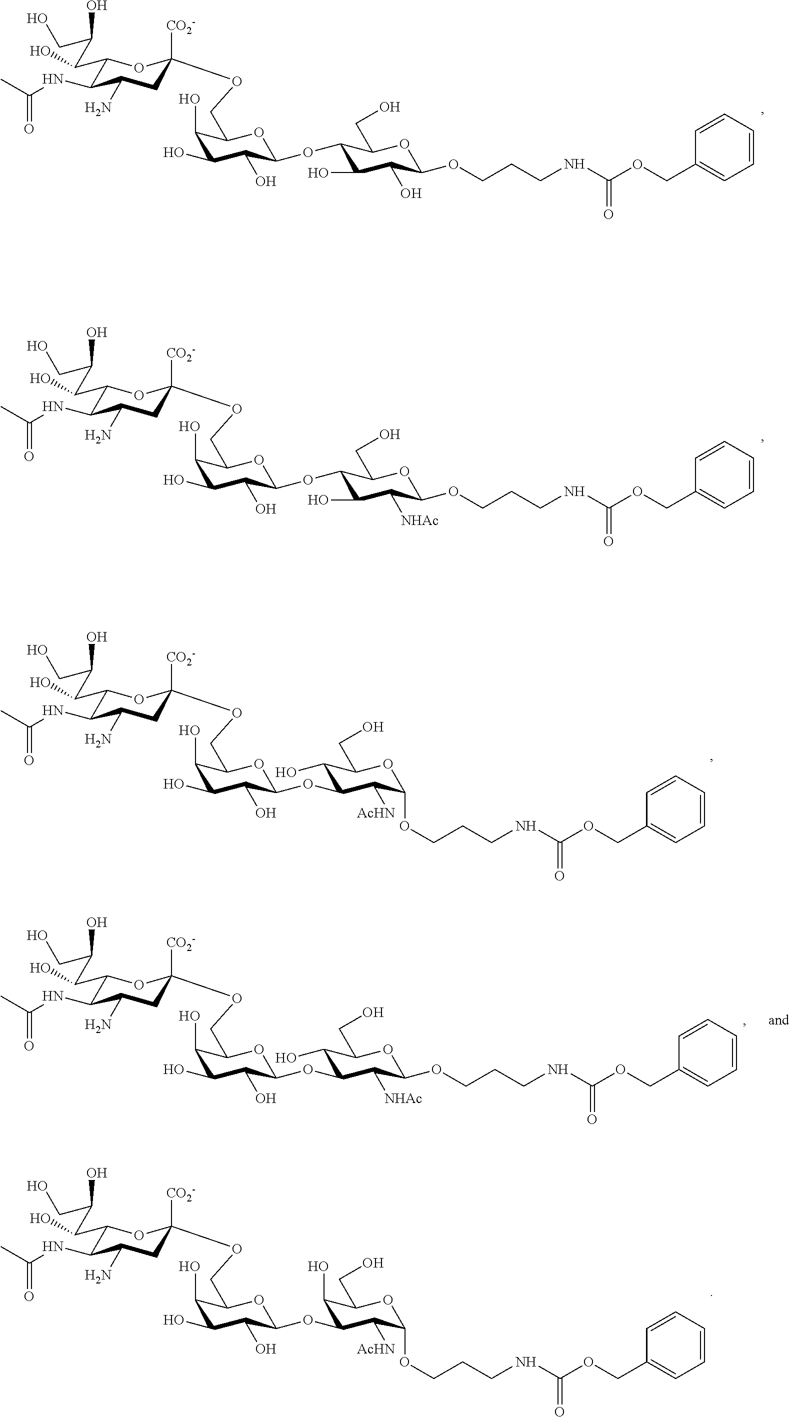

Also provided herein is a kit for determining the presence of neuraminidase, including an α2-6-linked 4NAcNeu5Ac-sialoside, or pharmaceutically acceptable salt thereof, selected from the group consisting of

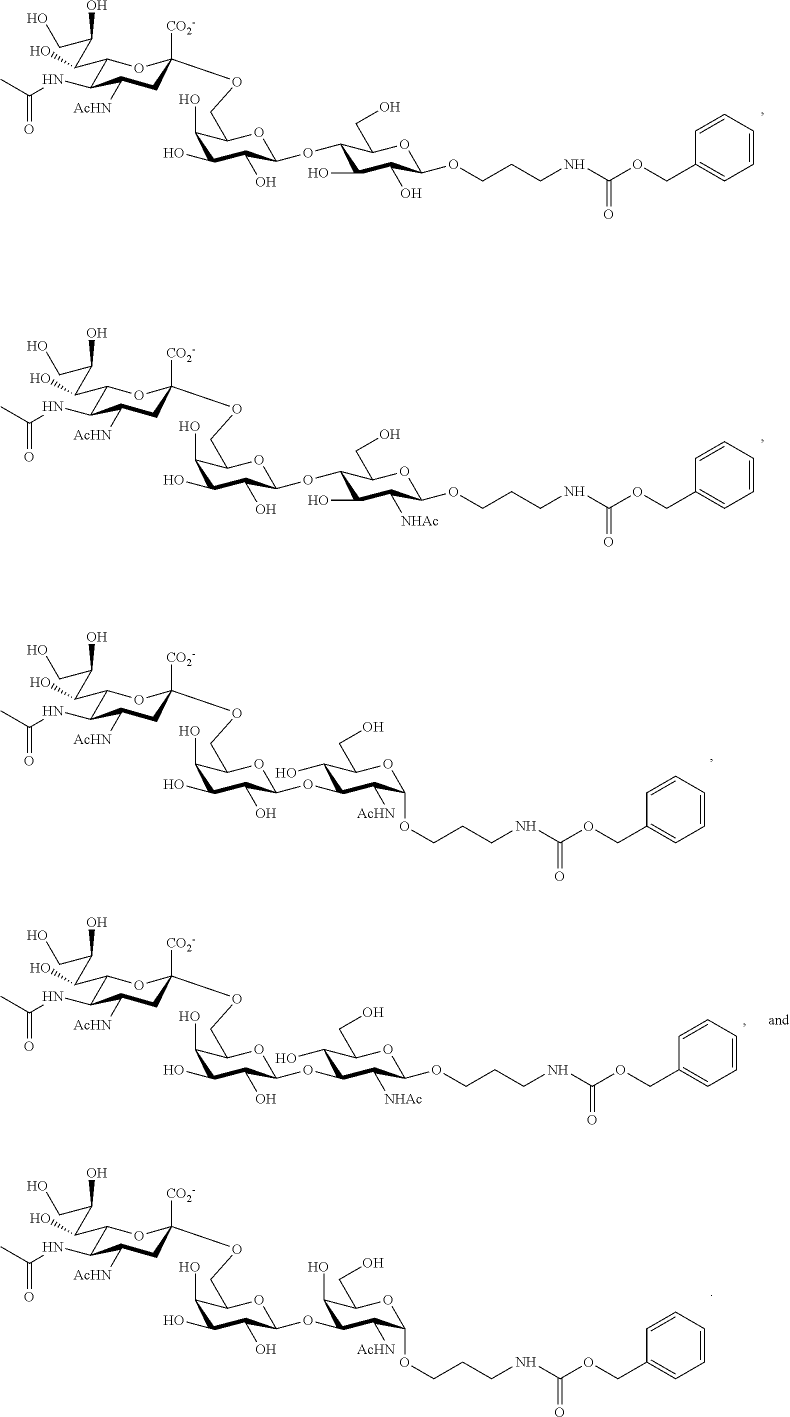

Also provided herein is a kit for determining the presence of neuraminidase, including a 4-methylumbelliferyl (MU)-tagged sialoside, or pharmaceutically acceptable salt thereof, selected from the group consisting of

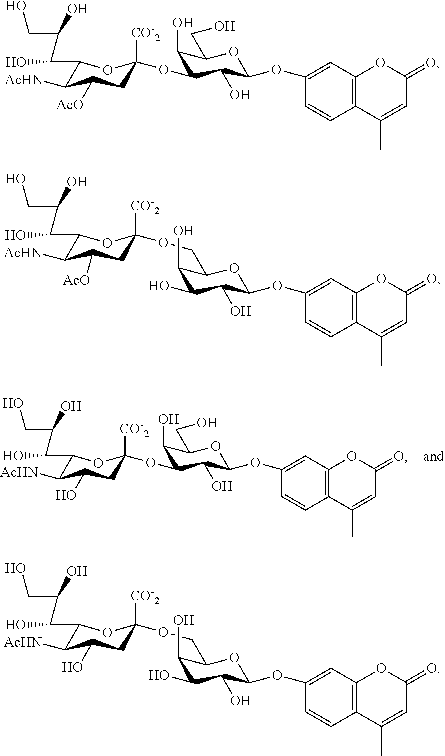

Also provided herein is a kit for determining the presence of neuraminidase, including a compound of Formula III:

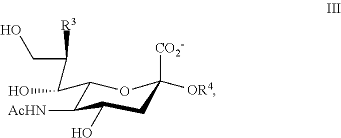

or a pharmaceutically acceptable salt thereof, wherein R³ is NH₂, NHAc, or N₃, and wherein R⁴ is H, C₁-C₆ alkyl, C₂-C₆ alkenyl, C₂-C₆ alkynyl, 4-methylumbelliferone (MU), para-nitrophenyl (pNP), indole, a monosaccharide, an oligosaccharide, a glycolipid, a glycopeptide, a glycoprotein, a galactoside, GalβMU, GalβpNP, or Galβ1-4GlcNAcβOR. In some examples, R³ is NH₂.

In some examples, the compounds of the kits described herein are substrates for neuraminidases, including, but not limited to, human neuraminidases, bacteria neuraminidases, influenza A virus neuraminidases, and/or influenza B virus neuraminidases. In some examples, the compound of the kits described herein is a selective substrate for influenza A neuraminidases. In some examples, the compound of the kit described herein is a selective substrate for influenza B neuraminidases.

### IX. Methods for Isolating and/or Purifying Neuraminidases

Also provided herein is a method of isolating a neuraminidase, including contacting the neuraminidase with a compound of Formula I:

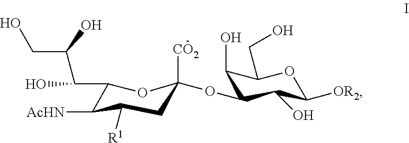

or a pharmaceutically acceptable salt thereof, wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, and ProNH-PEG4-biotin. In some examples, R¹ is NH₂. In some examples, R¹ is NH₂ or guanidine. In some examples, R¹ is N₃.

In some examples, the compound, or pharmaceutically acceptable salt thereof, for use in the methods described herein is selected from the group consisting of:

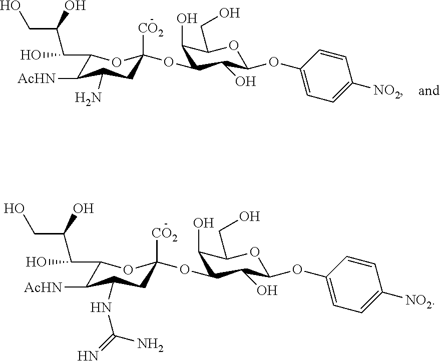

Also provided herein is a method of isolating a neuraminidase, including contacting the neuraminidase with a compound of Formula II:

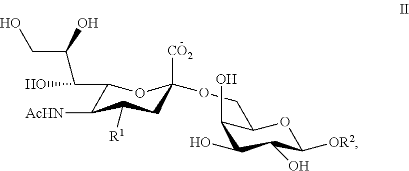

or a pharmaceutically acceptable salt thereof, wherein R¹ is selected from the group consisting of NH₂, NHAc, guanidine, and N₃, and R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, ProNH-Biotin, and ProNH-PEG4-biotin. In some examples, R¹ is NH₂ or guanidine.

In some examples, the compound for use in the methods described herein includes a compound selected from the group consisting of:

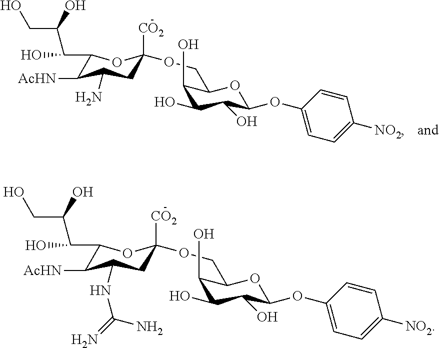

Also provided herein is a method of isolating a neuraminidase, including contacting the neuraminidase with a biotinylated compound, or pharmaceutically acceptable salt thereof, selected from the group consisting of

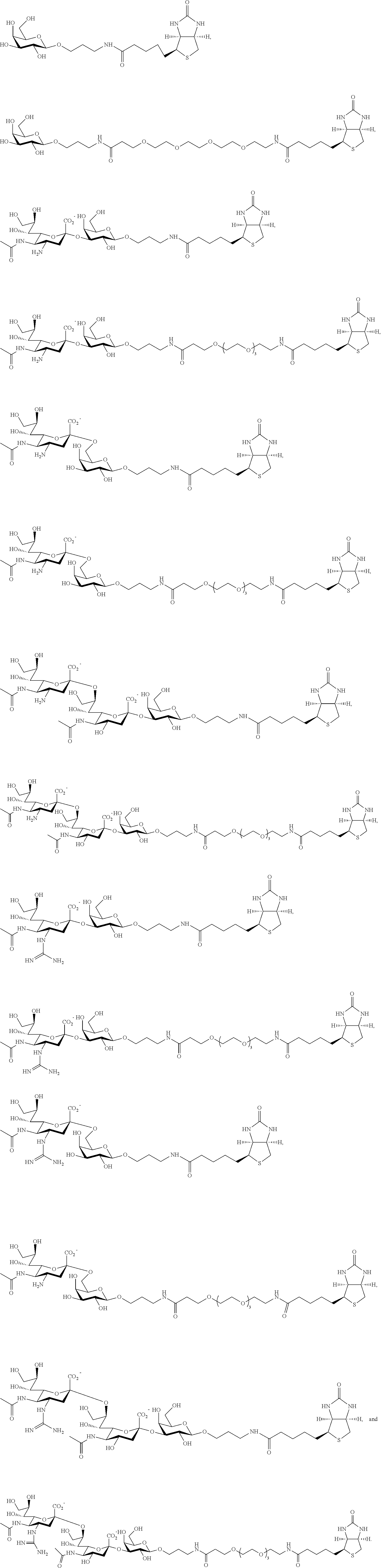

Also provided herein is a method of isolating a neuraminidase, including contacting the neuraminidase with a biotinylated compound, or pharmaceutically acceptable salt thereof, selected from the group consisting of Neu5 Acα2-3GalβMU, GalβProNH-PEG4-Biotin, Neu5Acα2-3 GalβProNH-PEG4-Biotin, Neu5Acα2-6GalβProNH-PEG4-Biotin, 4NH₂Neu5 Acα2-3 GalβProNH-PEG4-Biotin, 4-Guanidino-Neu5 Acα2-3GalβProNH-PEG4-Biotin, 4N₃Neu5Acα2-3GalβProNH-PEG4-Biotin, 4NAcNeu5Acα2-3GalβProNH-PEG4-Biotin, LacNAcProNH-PEG4-Biotin, Neu5Acα2-3LacNAcProNH-PEG4-Biotin, Neu5Acα2-6LacNAcProNH-PEG4-Biotin, 4NH₂Neu5Acα2-3LacNAcProNH-PEG4-Biotin, 4-Guanidino-Neu5Acα2-3LacNAcProNH-PEG4-Biotin, 4N₃Neu5Acα2-3LacNAcProNH-PEG4-Biotin, and 4NAcNeu5Acα2-3LacNAcProNH-PEG4-Biotin. In some examples, the biotinylated compound, or pharmaceutically acceptable salt thereof, selected from the group consisting of 4NH₂Neu5Acα2-3GalβProNH-PEG4-Biotin, 4-Guanidino-Neu5 Acα2-3GalβProNH-PEG4-Biotin, 4N₃Neu5 Acα2-3GalβProNH-PEG4-Biotin, 4NAcNeu5 Acα2-3GalβProNH-PEG4-Biotin, 4NH₂Neu5Acα2-3LacNAcProNH-PEG4-Biotin, 4-Guanidino-Neu5Acα2-3LacNAcProNH-PEG4-Biotin, 4N₃Neu5Acα2-3LacNAcProNH-PEG4-Biotin, and 4NAcNeu5Acα2-3LacNAcProNH-PEG4-Biotin. In some examples, the biotinylated compound, or pharmaceutically acceptable salt thereof, selected from the group consisting of 4NH₂Neu5Acα2-3 GalβProNH-PEG4-Biotin, 4-Guanidino-Neu5Acα2-3GalβProNH-PEG4-Biotin, 4NH₂Neu5Acα2-3LacNAcProNH-PEG4-Biotin, and 4-Guanidino-Neu5Acα2-3LacNAcProNH-PEG4-Biotin.

Also provided herein is a method of isolating a neuraminidase, including contacting the neuraminidase with an α2-3-linked 4NH₂Neu5Ac-sialoside, or a pharmaceutically acceptable salt thereof, selected from the group consisting of

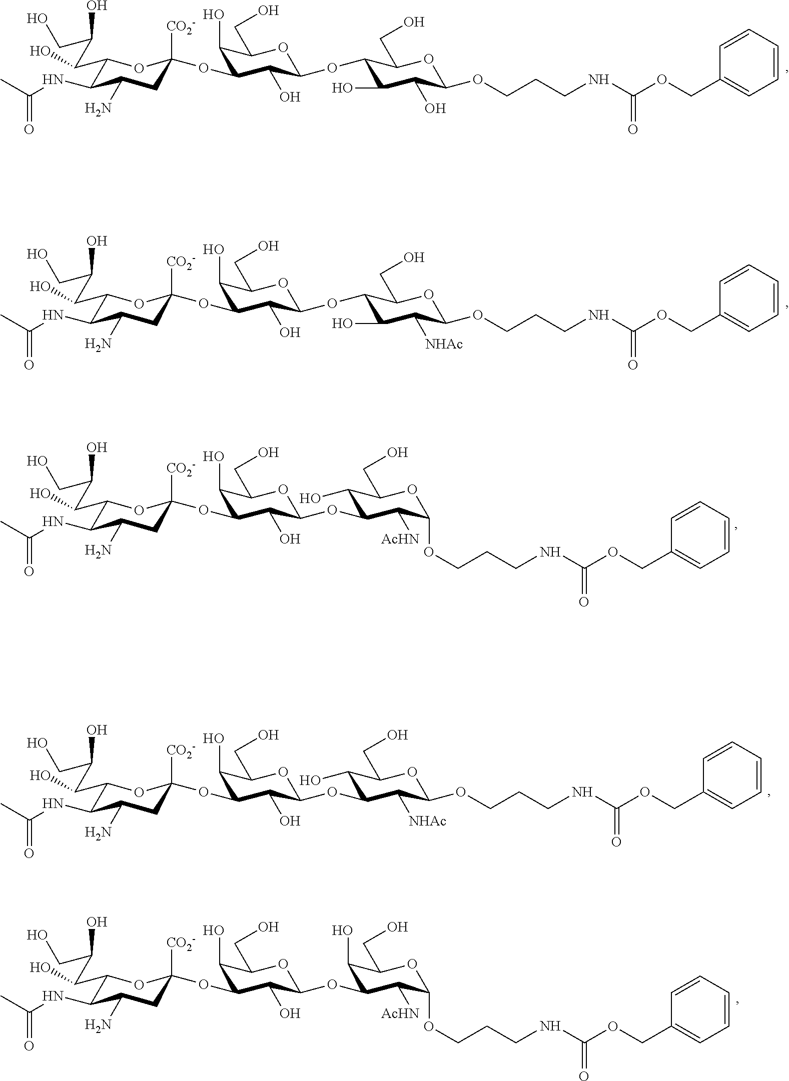

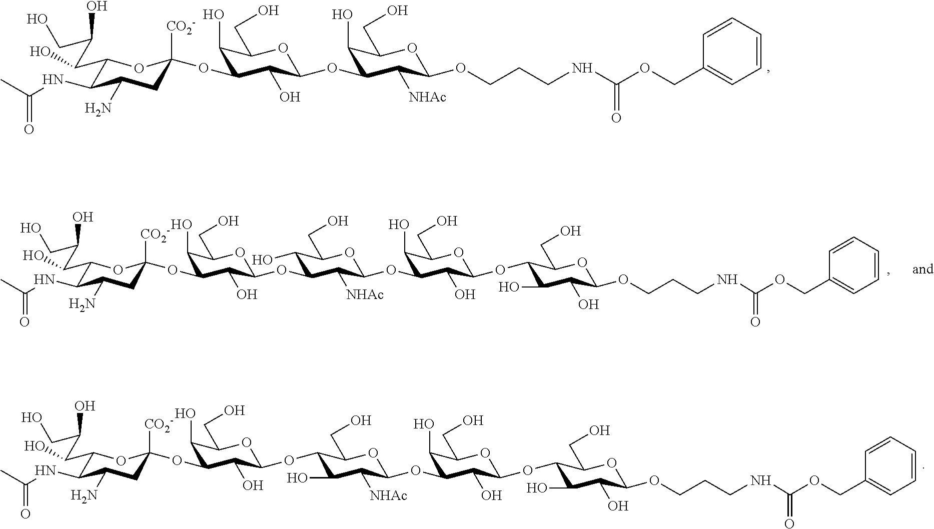

Also provided herein is a method of isolating a neuraminidase, including contacting the neuraminidase with an α2-3-linked 4NAcNeu5Ac-sialoside, or a pharmaceutically acceptable salt thereof, selected from the group consisting of

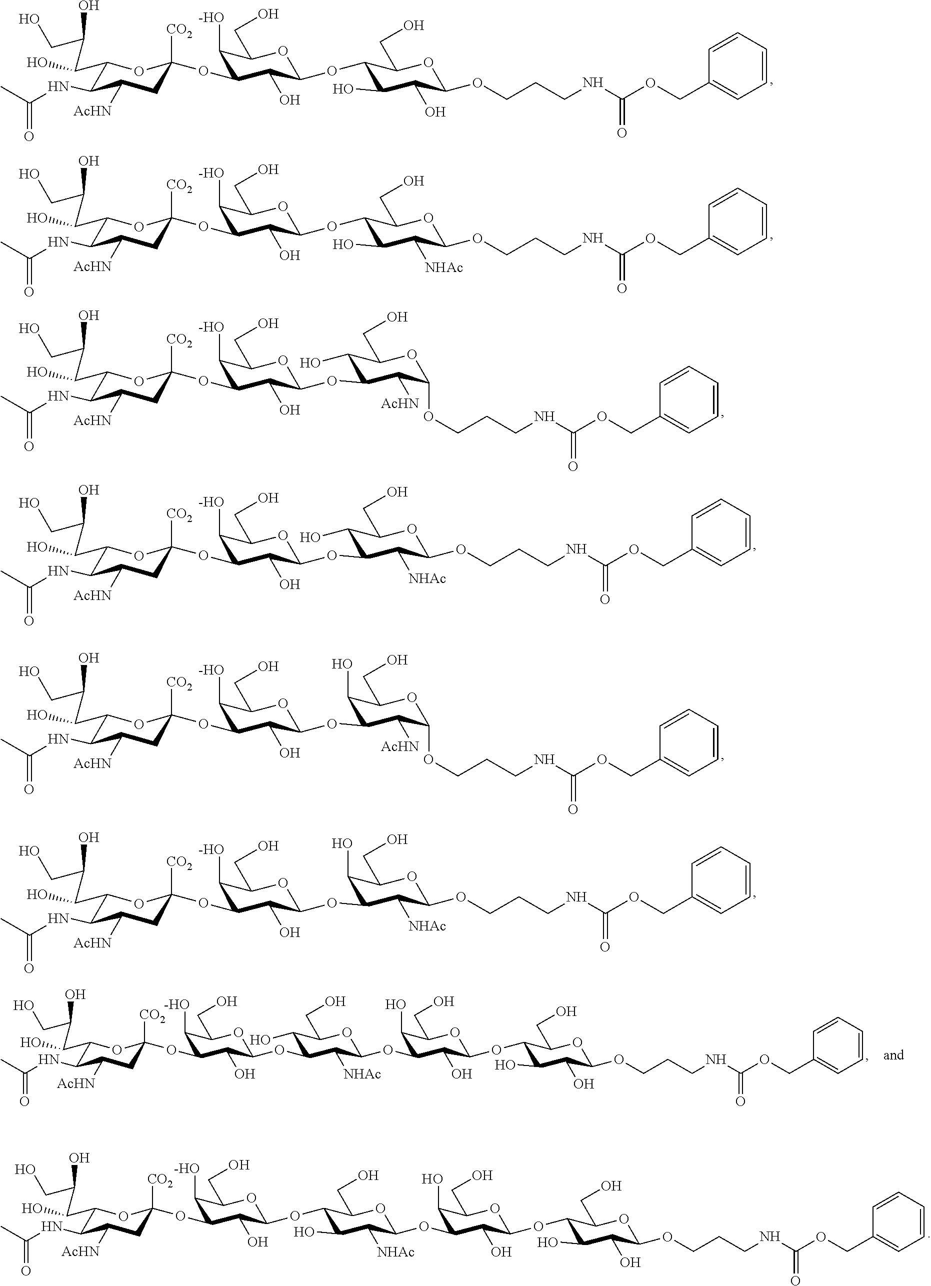

Also provided herein is a method of isolating a neuraminidase, including contacting the neuraminidase with an α2-6-linked 4NH₂Neu5Ac-sialoside, or a pharmaceutically acceptable salt thereof, selected from the group consisting of

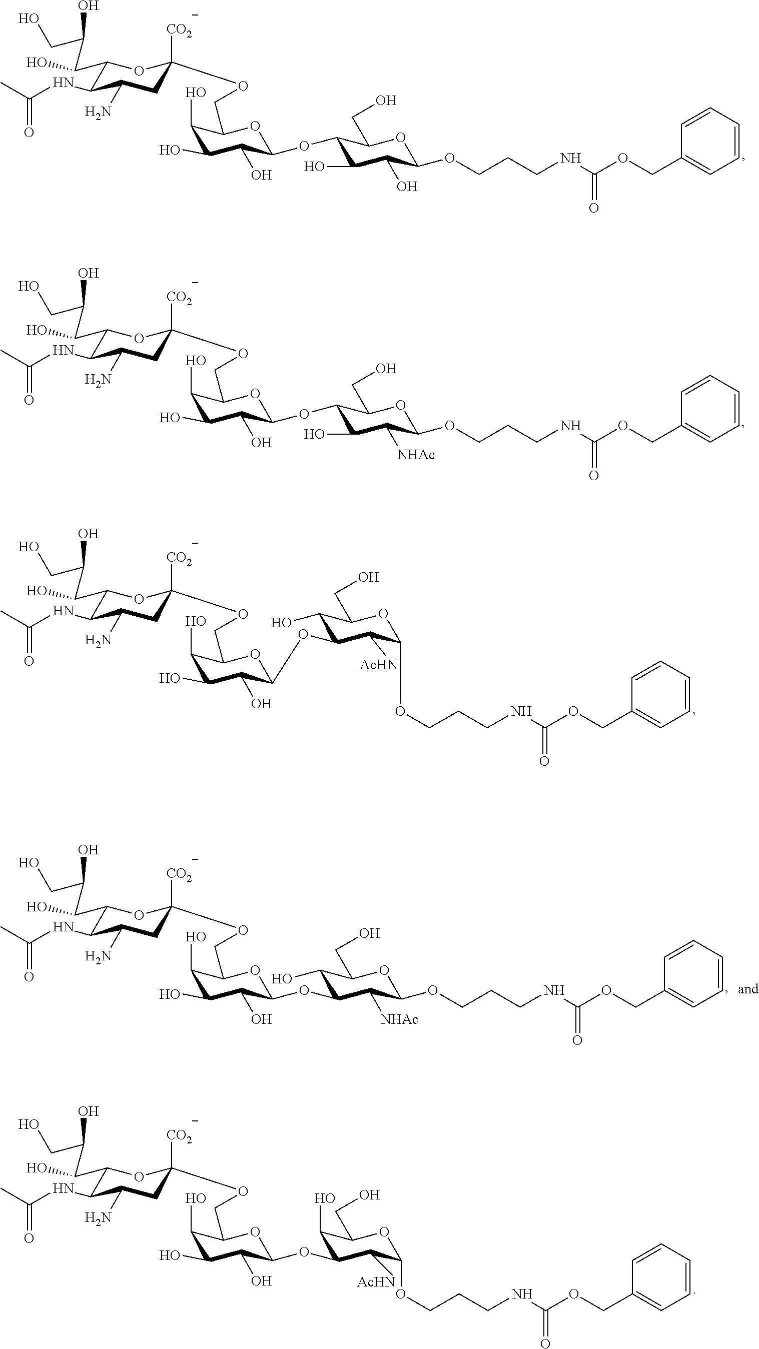

Also provided herein is a method of isolating a neuraminidase, including contacting the neuraminidase with an α2-6-linked 4NAcNeu5Ac-sialoside, or a pharmaceutically acceptable salt thereof, selected from the group consisting of

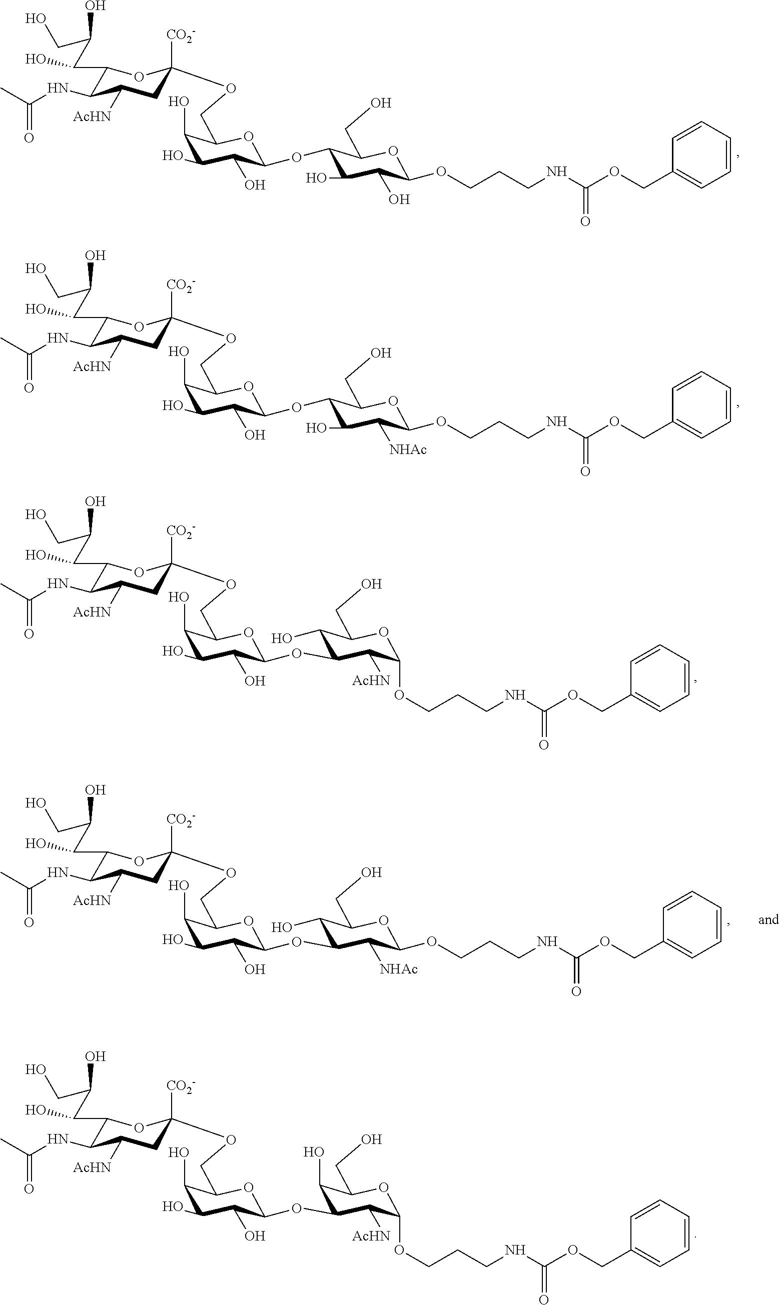

Also provided herein is a method of isolating a neuraminidase, including contacting the neuraminidase with 4-methylumbelliferyl (MU)-tagged sialoside, or a pharmaceutically acceptable salt thereof, selected from the group consisting of

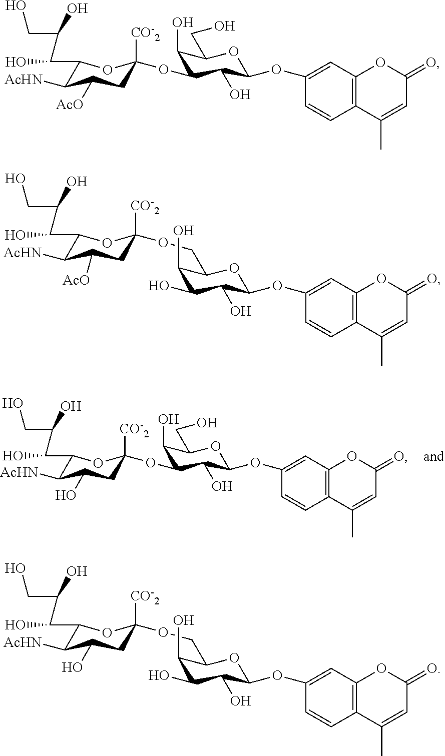

Also provided herein is a method of isolating a neuraminidase, including contacting a neuraminidase with a compound of Formula III:

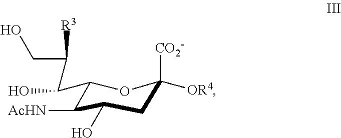

or a pharmaceutically acceptable salt thereof, wherein R³ is NH₂, NHAc, or N₃, and wherein R⁴ is H, C₁-C₆ alkyl, C₂-C₆ alkenyl, C₂-C₆ alkynyl, 4-methylumbelliferone (MU), para-nitrophenyl (pNP), indole, a monosaccharide, an oligosaccharide, a glycolipid, a glycopeptide, a glycoprotein, a galactoside, GalβMU, GalβpNP, or Galβ1-4GlcNAcβOR. In some examples, R³ is NH₂.

The compounds of the methods described herein can isolate neuraminidases including, but not limited to, human neuraminidases, bacteria neuraminidases, and influenza virus neuraminidases. In some examples, the compounds of the methods described herein can isolate one or more neuraminidases such that the one or more neuraminidases can be obtained in a purity from at least 30 percent to greater than 90 percent for one or more neuraminidases (e.g., at least 30 percent, at least 40 percent, at least 50 percent, at least 60 percent, at least 70 percent, at least 80 percent, at least 90 percent, or greater than 90 percent). In some examples, the compounds of the methods described herein can isolate influenza A virus neuraminidases and/or influenza B virus neuraminidases. In some examples, the compounds of the methods described herein selectively isolate influenza A virus neuraminidases and/or influenza B neuraminidases. In some examples, the compounds of the methods described herein selectively isolate influenza A virus neuraminidases. In some examples, compounds of the methods described herein selectively isolate influenza B virus neuraminidases.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1. 4—N-Sialoside Substrate Specificity for Influenza Virus Neuraminidases

The numbering of compounds, schemes, and tables presented in this Example 1 is specific only to this Example 1 and Example 2 below. Compounds as described herein were used together with Neu5Acα2-3GalβpNP, Neu5Acα2-6GalβpNP, and Neu4, 5Ac₂α2-3GalβpNP as substrates (Table 1) for high-throughput substrate specificity studies of human, bacterial, and viral NAs.

TABLE 1
Example compounds tested for substrate specificity for human, bacterial, and viral NAs.

| Compound | Compound Name |
|---|---|
| 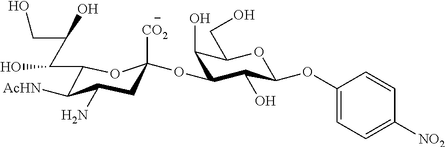 | 4NH₂Neu5Acα2-3GalβpNP |
| 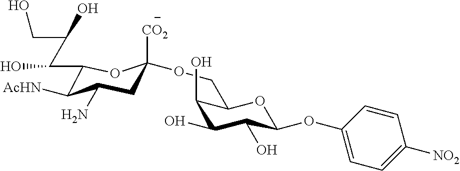 | 4NH₂Neu5Acα2-6GalβpNP |
| 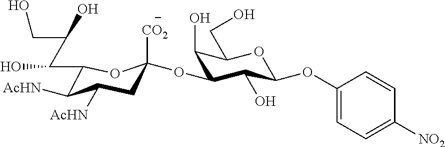 | 4NAcNeu5Acα2-3GalβpNP |
| 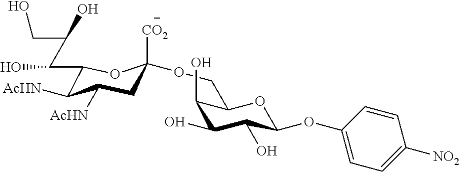 | 4NAcNeu5Acα2-6GalβpNP |
| 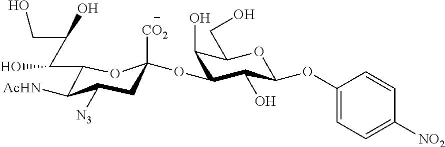 | 4N3Neu5Acα2-3GalβpNP |
| 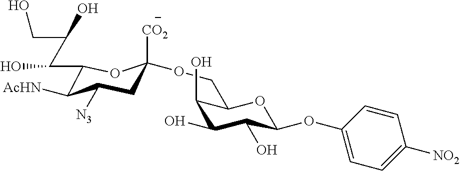 | 4N3Neu5Acα2-6GalβpNP |
| 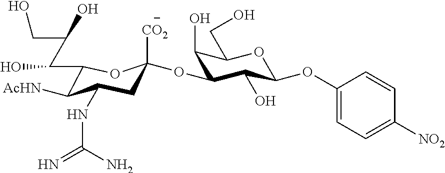 | 4-Guanidino-Neu5Acα2-3GalβpNP |
| 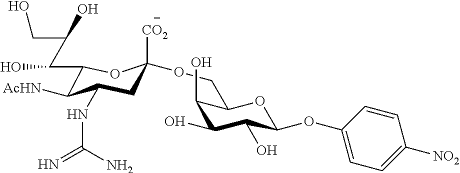 | 4-Guanidino-Neu5Acα2-6GalβpNP |
| 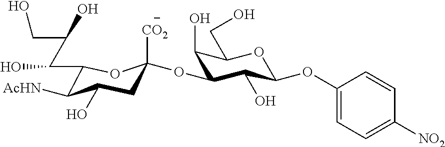 | Neu5Acα2-3GalβpNP |
| 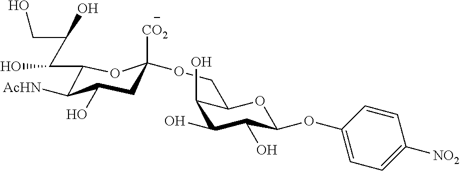 | Neu5Acα2-6GalβpNP |
| 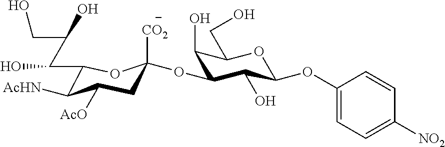 | Neu4,5Acα2-3GalβpNP |

To compare relative substrate preferences, the concentrations of NAs were determined so that the cleavage of Neu5 Acα2-3GalβpNP during 30 minutes of reaction time at 37° C. reached 40-80% to obtain the “low-concentration” condition of the enzymes. Each sialoside, in a 384-well plate, was incubated with an NA in the presence of an excess amount of β-galactosidase which catalyzed the GalβpNP that is released from a sialoside by an active NA to form p-nitrophenol. Upon the addition of N-cyclohexyl-3-aminopropanesulfonic acid (CAPS) buffer to adjust the pH to above 9.5 at the end of the reaction time, A_{405 nm} values were determined using a plate reader.

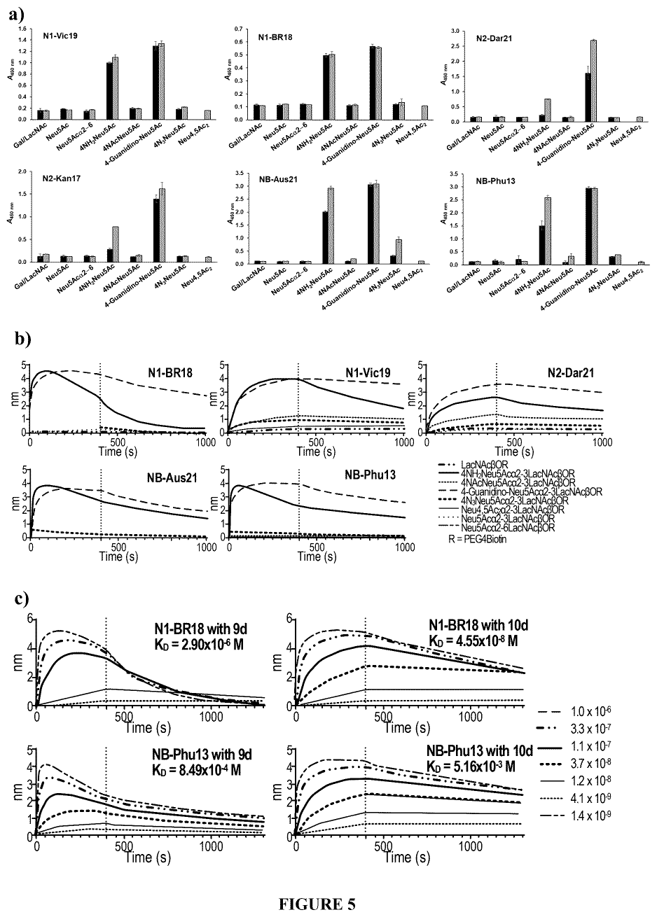

As shown in FIG. 4, under “low-concentration” enzyme conditions, 4N₃Neu5Acα2-3 GalβpNP and Neu4,5Ac₂α2-3GalβpNP were cleaved off selectively by influenza virus NAs only.

Sialidase substrate specificity assays were further carried out with a longer reaction time (1 hour) and with 4-fold (for Vc Sialidase) or 10-fold higher concentrations (for other NAs) of NAs (to obtain the “high-concentration” condition of the enzymes). As shown in FIG. 4, 4N₃Neu5Acα2-3GalβpNP and Neu4,5Ac₂α2-3GalβpNP were completely cleaved under these conditions, again selectively by influenza virus NAs only. A low percentage of 4N₃Neu5Acα2-6GalβpNP was also selectively cleaved off selectively by influenza virus neuraminidases. When the “high-concentration” enzyme reactions were carried out for an even longer reaction time (14 hours), the selective cleavages of 4N₃Neu5 Acα2-3/6GalβpNP, 4-guanidino-Neu5Acα2-3/6GalβpNP, and Neu4, 5Ac₂α2-3 GalβpNP selectively by influenza virus NAs were observed (FIG. 4). While 4NH₂Neu5Acα2-3/6GalβpNP were weakly cleaved off by both SpNanA and some influenza virus NAs, 4NAcNeu5 Acα2-3GalβpNP was weakly cleaved off by only influenza virus N1-Vic19.

### Example 2. Inhibition of Influenza Neuraminidase with 4-N-Sialosides

The numbering of compounds, schemes, and tables presented in this Example 2 is specific only to Example 1 above and this Example 2. Both 4NH₂Neu5Ac2en and 4-guanidino-Neu5Ac2en (or Zanamivir) bond to influenza virus NAs much stronger than Neu5Ac2en. Both 4NH₂Neu5Acα2-3/6GalβpNP and 4-guanidino-Neu5Acα2-3/6GalβpNP being poor substrates for the influenza virus NAs tested indicates that these sialosides could be efficient inhibitors.

Herein, a fluorescence-based inhibition assay platform is described to test for influenza virus NA inhibition. In this assay, a 4-methylumbelliferyl (MU)-tagged sialoside Neu5Acα2-3GalβMU (8 μM) was used as a sialidase substrate in a 96-well plate. In the absence of an inhibitor, sialidase catalyzes the cleavage of Neu5 Ac to form GalβMU which is cleaved by the excess amount of β-galactosidase added in the assay to release MU, of which the fluorescence is detected by a plate reader.

Indeed, when Neu5 Acα2-3GalβMU (8.0 μM) was used as the substrate and the same concentration of a pNP-tagged sialoside was used as an inhibitor, 4NH₂Neu5Acα2-3GalβpNP, 4NH₂Neu5Acα2-6GalβpNP, 4-guanidino-Neu5 Acα2-3GalβpNP, and 4-guanidino-Neu5Acα2-6GalβpNP showed 61%, 15%, 7%, and 90% inhibitory activities, respectively, against IAV neuraminidase N1-BR18 (Table 2). Compounds 4NH₂Neu5 Acα2-3GalβpNP, 4NH₂Neu5Acα2-6GalβpNP, 4-guanidino-Neu5Acα2-3GalβpNP, and 4-guanidino-Neu5Acα2-6GalβpNP showed 45%, 36%, 86%, and 69% inhibitory activities, respectively, against IBV neuraminidase Aus-21 (Table 2). In contrast, none of the pNP-tagged sialosides showed any inhibitory activity against hNEU2 under same assay conditions.

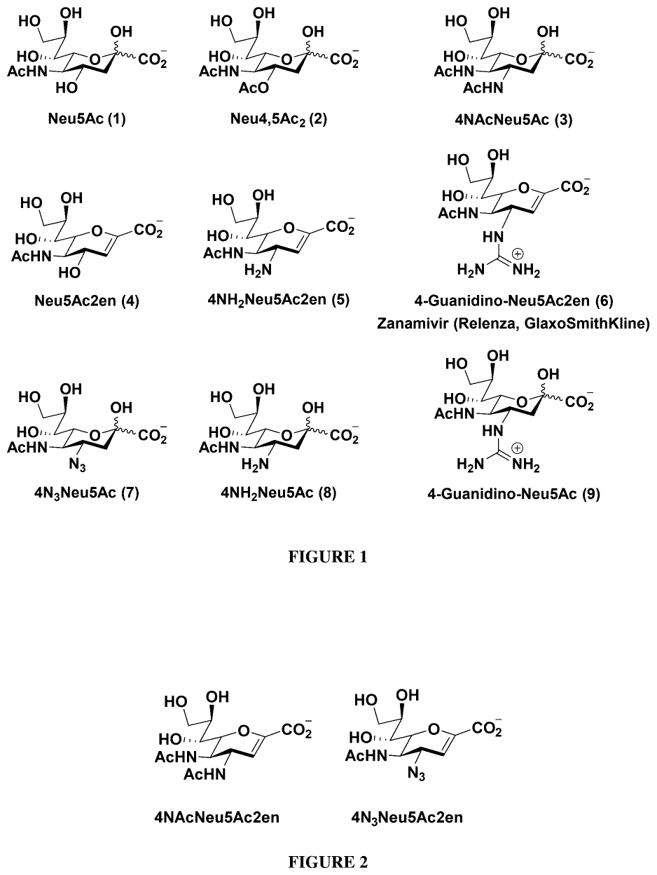

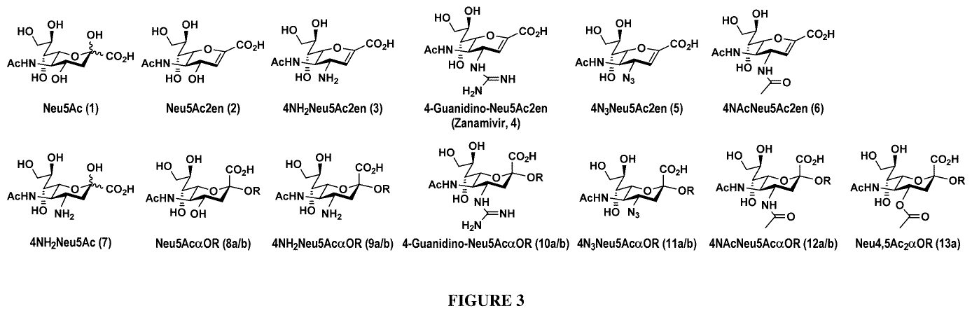

The inhibitory activities of several Sia2en compounds and sialic acid derivatives were also assayed and compared (Table 2). 4NH₂Neu5Ac2en and Zanamivir showed better inhibitory activities against N1-BR18 or NB-Aus21 compared to Neu5Ac2en. The inhibitory activities of 4N₃Neu5Ac2en (FIG. 2) and 4NH₂Neu5Ac, and 4-guanidino-Neu5Ac against N1-BR18 and NB-Aus21 were also observed. Both Neu5Ac2en and Zanamivir also showed inhibitory activities against hNEU2 but were weaker compared to those against N1-BR18 and NB-Aus21.

TABLE 2
Percentage inhibition of pNP-tagged sialosides containing
a 4-N-modified Neu5Ac, Sia2en compounds, and Sia derivatives
against IAV NI-BR18, IBV NB-Aus21, and hNEU2.

| | NA | | |
|---|---|---|---|
| | N1-BR18 | NB-Aus21 | |
| Inhibitor | (IAV) | (IBV) | hNEU2 |
| Neu5Acα2-3GalβpNP | 2.3 ± 1.2 | 25.6 ± 2.3 | 0.0 ± 5.0 |
| Neu5Acα2-6GalβpNP | 0.0 ± 4.0 | 25.8 ± 2.1 | 0.0 ± 6.7 |
| 4NH₂Neu5Acα2-3GalβpNP | 60.6 ± 1.7 | 45.3 ± 0.1 | 0.0 ± 2.0 |
| 4NH₂Neu5Acα2-6GalβpNP | 14.9 ± 13.4 | 35.5 ± 3.8 | 0.0 ± 2.5 |
| 4NAcNeu5Acα2-3GalβpNP | 3.2 ± 3.2 | 17.3 ± 1.2 | 0.0 ± 2.3 |
| 4NAcNeu5Acα2-6GalβpNP | 0.0 ± 0.9 | 21.5 ± 1.7 | 0.0 ± 1.7 |
| 4-Guanidino-Neu5Acα2-3GalβpNP | 97.0 ± 0.0 | 85.8 ± 0.7 | 0.0 ± 0.6 |
| 4-Guanidino-Neu5Acα2-6GalβpNP | 90.1 ± 1.1 | 69.4 ± 1.3 | 0.0 ± 3.2 |
| 4N₃Neu5Acα2-3GalβpNP | 0.0 ± 1.2 | 22.0 ± 3.7 | 0.0 ± 1.9 |
| 4N₃Neu5Acα2-6GalβpNP | 0 ± 3.4 | 23.0 ± 3.6 | 0.0 ± 3.6 |
| Neu4,5Ac₂α2-3GalβpNP | 0 ± 6.4 | 20.9 ± 4.2 | 0.0 ± 7.0 |
| Neu5Ac2en | 60.8 ± 16.4 | 82.6 ± 1.7 | 37.1 ± 1.1 |
| 4NH₂Neu5Ac2en | 93.6 ± 0.2 | 98.3 ± 0.1 | 0.0 ± 6.6 |
| Zanamivir | 98.5 ± 0.1 | 99.9 ± 0.0 | 26.0 ± 3.6 |
| 4N₃Neu5Ac2en | 40.7 ± 2.7 | 82.8 ± 1.7 | 3.3 ± 0.6 |
| 4NAcNeu5Ac2en | 0.0 ± 2.5 | 21.3 ± 0.2 | 2.1 ± 2.5 |
| Neu5Ac (1) | 5.2 ± 5.3 | 20.2 ± 2.6 | 1.7 ± 0.5 |
| 4NAcNeu5Ac (3) | 11.5 ± 22.7 | 27.9 ± 10.1 | 3.0 ± 1.0 |
| 4N₃Neu5Ac (7) | 0.0 ± 4.5 | 37.5 ± 4.1 | 3.0 ± 0.8 |
| 4NH₂Neu5Ac (8) | 28.6 ± 24.5 | 41.8 ± 2.8 | 2.2 ± 1.2 |
| 4-Guanidino-Neu5Ac (9) | 76.4 ± 4.9 | 51.0 ± 0.1 | 3.3 ± 0.6 |

IC₅₀ values (μM) were also determined for some of the inhibitors against N1-BR18 (Table 3). The IC₅₀ values of Neu5Ac2en and zanamivir obtained were comparable with the values reported previously. The IC₅₀ values of 4-guanidino-Neu5Acα2-3GalβpNP and 4-guanidino-Neu5Acα2-6GalβpNP against N1-BR18 (0.11 μM and 0.74 μM, respectively) were comparable to that of 4NH₂Neu5 Ac2en (0.49 μM).

TABLE 3
IC₅₀ values (μM) of inhibitors determined by the fluorescence-based inhibition assay.

| | | | | 4-Guanidino- | 4-Guanidino- |
|---|---|---|---|---|---|
| | | | | Neu5Acα2- | Neu5Acα2- |
| NAs | Neu5Ac2en | 4NH₂Neu5Ac2en | Zanamivir | 3GalβpNP | 6GalβpNP |
| N1-BR18 | 4.5 | (4.9 ± 2.2) × 10⁻¹ | 4.7 × 10⁻³ | (1.1 ± 0.5) × 10⁻¹ | (7.4 ± 5.8) × 10⁻¹ |

### Example 3. 4—N-Derivatized Sialyl Glycosides for Influenza Virus Neuraminidase Detection and Purification

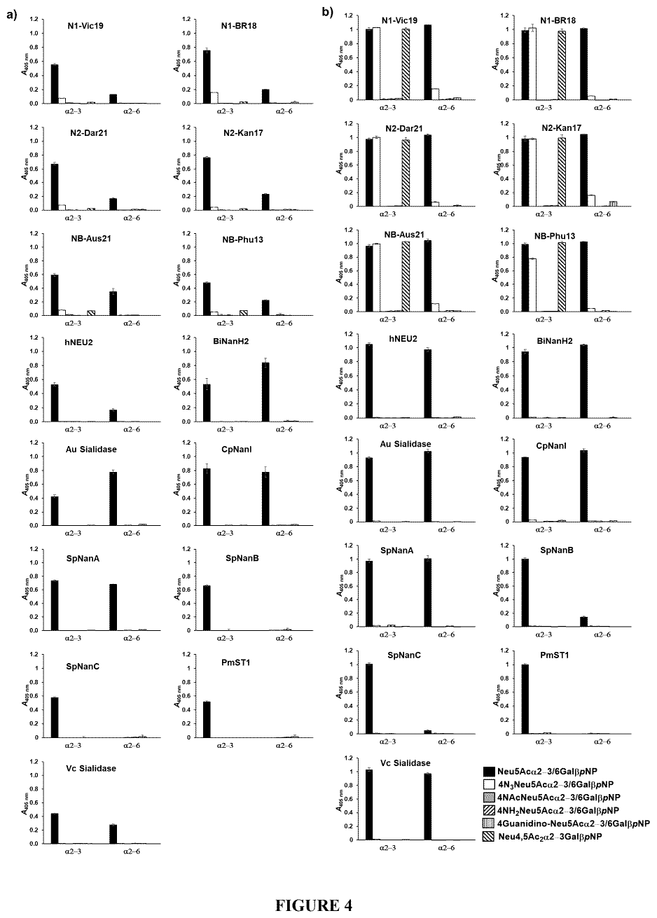

The numbering of compounds, schemes, and tables presented in this Example 3 is specific only to this Example 3. FIG. 3 shows the following compounds: N-acetylneuraminic acid (Neu5Ac, 1); 2,3-dehydro-3-deoxy-Neu5Ac (Neu5Ac2en, 2) and its 4-N-substituted derivatives (3-6); 4-amino-Neu5Ac (4NH₂Neu5Ac, 7), α2-3 and α2-6-linked Neu5Ac-terminated α-sialosides (Neu5AcαOR, 8a/b) (R=GalβpNP) and their derivatives containing a 4-N-substituted Neu5Ac (9a/b-12a/b); and α2-3-linked 4-O-acetyl Neu5 Ac-terminated α-sialoside (Neu4,5Ac₂αOR, 13a).

4NH₂Neu5Ac (7) was synthesized from Neu5Ac (1) in seven steps via a fully protected 4-azido-Neu5Ac2en intermediate (16) (Scheme 1a). Briefly, the carboxyl group in Neu5Ac (1) was methylated and its hydroxyl groups were acetylated to form per-O-acetylated methyl ester 14 in two steps in an overall 90% yield. Treatment of 14 with BF₃·Et₂O formed oxazoline 15, which reacted with trimethylsilyl azide (TMSN₃) to produce 4-azido methyl ester 16. Treatment of 16 with N-bromosuccinimide (NBS) followed by debromination and reduction using tri-n-butyltinhydride yielded 4-amino methyl ester 17 in 42% yield over two steps. Deprotection of 17 with sodium methoxide in methanol and water formed the desired 4NH₂Neu5 Ac (7).

The protected 4-azido methyl ester 16 was also used as an intermediate for synthesizing 4-N-derivatized Neu5 Ac2en including 4N₃Neu5 Ac2en (5), 4NH₂Neu5Ac2en (3), and 4NAcNeu5Ac2en (6). Briefly, deprotection of 16 in NaOH aqueous solution formed 4N₃Neu5Ac2en (5) in 95% yield. Treating 5 with PMe₃ under a basic condition produced 4NH₂Neu5Ac2en (3) in 86% yield. Incubating 16 with thioacetic acid (AcSH) in pyridine followed by deprotection with NaOH in MeOH produced 4NAcNeu5 Ac2en (6) in 94% yield.

After synthesizing 4NH₂Neu5Ac (7), para-nitrophenol (pNP)-tagged sialosides 4NH₂Neu5Acα2-3GalβpNP (9a) and 4NH₂Neu5Acα2-6GalβpNP (9b) (Scheme 1b) were prepared in 92% and 82% yields, respectively, using a one-pot two-enzyme (OP2E) sialylation systems containing Neisseria meningitidis cytidine 5′-monophosphate (CMP)-sialic acid synthetase (NmCSS) and Pasteurella multocida α2-3-sialyltransferase-1 (PmST1) or Photobacterium sp. «2-6-sialyltransferase A366G mutant (Psp2,6ST_A366G).

Scheme 1 (a) shows the chemical synthesis of 4NH₂Neu5Ac (7) and 4-N-substituted Neu5Ac2en including 4NH₂Neu5Ac2en (3), 4N₃Neu5Ac2en (5), and 4NAcNeu5 Ac2en (6) from commercially available Neu5Ac. Scheme 1 (b) shows the chemoenzymatic synthesis of 4NH₂Neu5Acα2-3GalβpNP (9a) and 4NH₂Neu5Acα2-6GalβpNP (9b) and other 4-N-derivatives including 4-guanidino-Neu5Acα2-3/6GalβpNP (10a/b), 4N₃Neu5Acα2-3/6GalβpNP (11a/b), and 4NAcNeu5Acα2-3/6GalβpNP (12a/b).

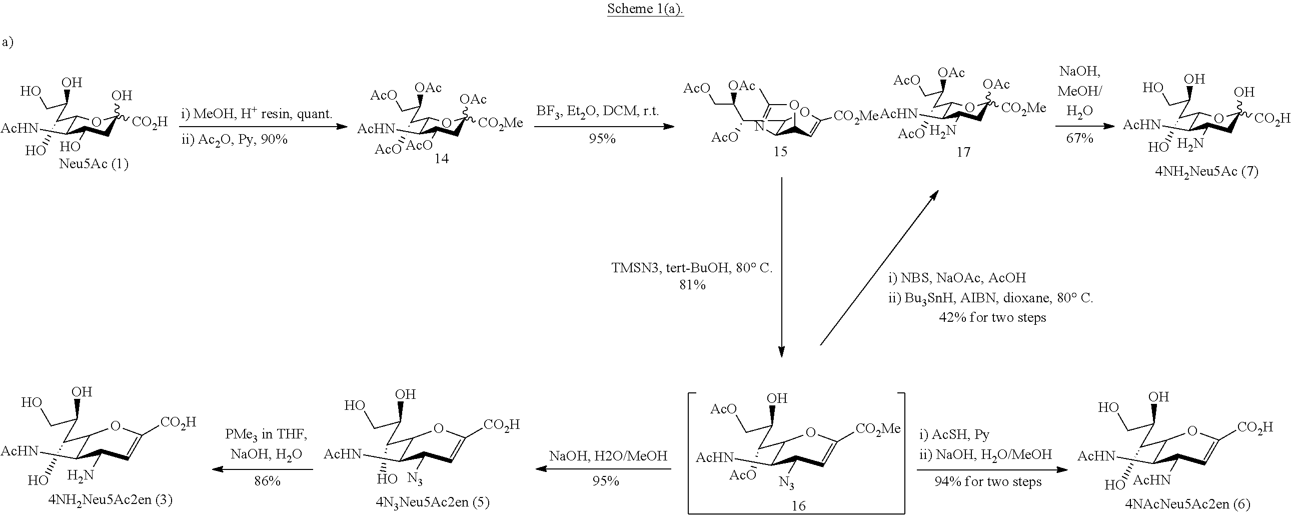

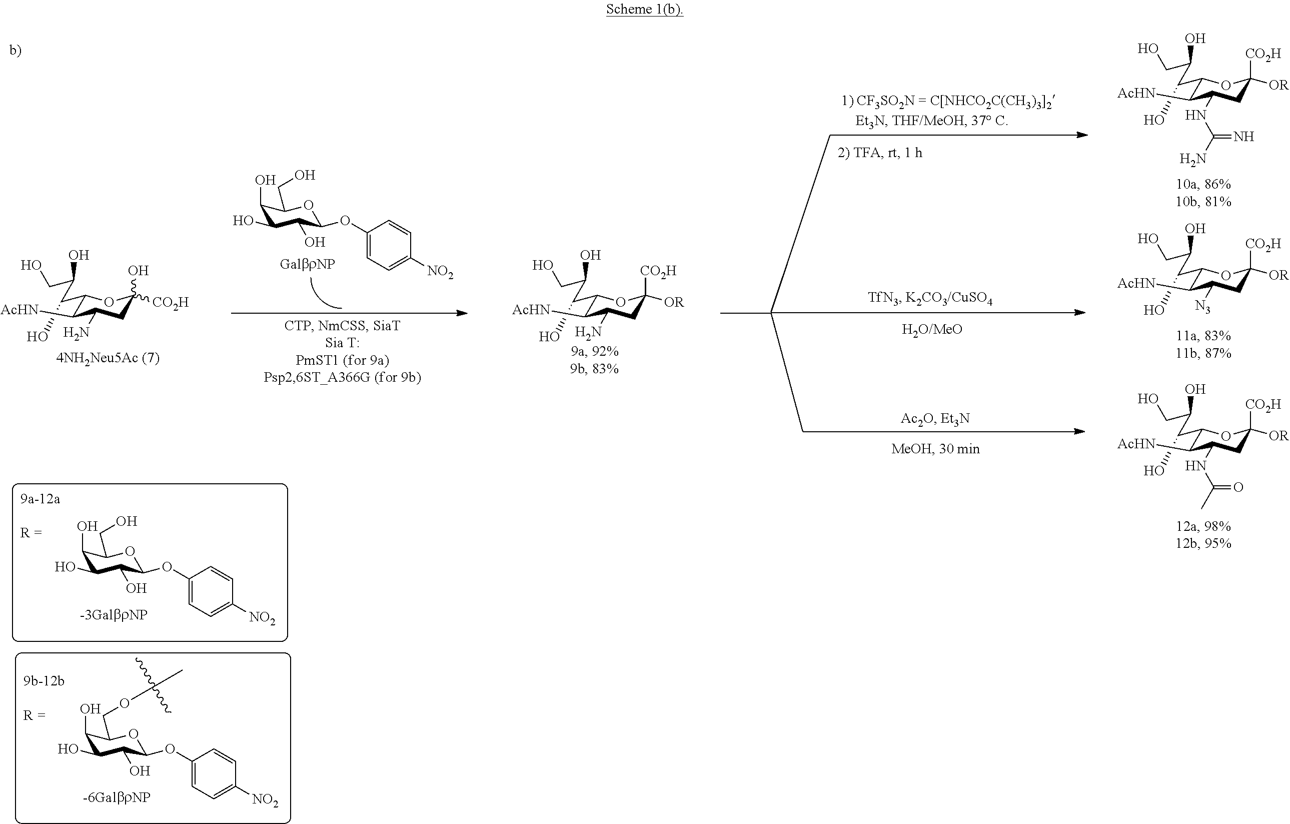

Derivatizing the 4—NH₂ group in 4NH₂Neu5Acα2-3/6GalβpNP (9a/b) formed other 4-N-modified sialosides (10a/b-12a/b) (Scheme 1b). Briefly, 4-guanidino-Neu5Acα2-3/6GalβpNP (10a/b) were obtained in 86% and 81% yields, respectively, by treating 9a/b with 1,3-di-Boc-2-(trifluoromethylsulfonyl) guanidine in tetrahydrofuran and methanol under basic reaction conditions followed by removing the Boc using trifluoroacetic acid (TFA). Treating 9a/b with freshly prepared triflate azide (TfN₃) in the presence of catalytic CuSO₄ and triethylamine produced 4N₃Neu5Acα2-3/6GalβpNP (11a/b) in 83% and 87% yields, respectively. Treating 9a/b with acetic anhydride in the presence of triethylamine produced 4NAcNeu5Acα2-3/6GalβpNP (12a/b) in 98% and 95% yields, respectively.

4N₃Neu5Acα2-3GalβpNP (11a), in addition to Neu4, 5Ac₂α2-3GalβpNP (13a), are Selective Substrates for Influenza NAs. The obtained sialosides containing 4-N-substituted Neu5Ac (9a/b-12a/b) were tested as potential substrates in a multiwell plate-based high-throughput assay for human, bacterial, and influenza sialidases. These include NAs from two recent H1N1 IAV strains A/Victoria/2570/2019 (N1-Vic19) and A/Brisbane/02/2018 (N1-BR18), and two H3N2 IAV strains A/Darwin/9/2021 (N2-Dar21) and A/Kansas/14/2017 (N2-Kan17); and NAs from two IBV strains B/Austria/1359417/2021 (NB-NB-Aus21) and B/Phuket/3073/2013 (NB-Phu13). Neu5Acα2-3/6GalβpNP (8a/b), and Neu4,5Ac₂α2-3GalβpNP (13a) containing a naturally occurring 4-O-acetylated Neu5 Ac selectively cleavable by influenza NAs were used as controls.

To compare relative substrate preferences, NA amounts in the low-enzyme-concentration assays were standardized so that Neu5 Acα2-3GalβpNP cleavage during a 30 minute reaction at 37° C. reached 40-80% but not higher. Each sialoside, in a 384-well plate, was incubated with an NA in the presence of an excess amount of β-galactosidase which catalyzes the cleavage of the NA product GalβpNP to produce p-nitrophenol. Following the addition of N-cyclohexyl-3-aminopropanesulfonic acid (CAPS) buffer (pH 11.5) to adjust the pH of the reaction mixture to above 9.5, A405 nm values (to quantify the formation of p-nitrophenolate) were determined using a plate reader.

FIG. 4 shows sialidase substrate specificity studies using sialyl GalβpNPs with panel (a) low (30 minutes) and panel (b) high (1 hour) enzyme-concentration assay conditions. Sialidase amounts for the low-enzyme-concentration assay conditions were standardized using the substrate Neu5Acα2-3GalβpNP. Sialidases in the high-enzyme-concentration assays were 10-fold higher than those in the low-enzyme-concentration assays except for Vc sialidase which was used at 4× of the low-enzyme-concentration assay conditions. The abbreviations in FIG. 4 are as follows: h, human; Bi, Bifidobacterium infantis; Au, Arthrobacter ureafaciens; Cp, Clostridium perfringens; Vc, Vibrio cholerae; Sp, Streptococcus pneumoniae; PmST1, the sialidase activity of Pasteurella multocida α2-3-sialyltransfearse 1 in the presence of CMP (0.4 mM).

As shown in FIG. 4, 4N₃Neu5 Acα2-3GalβpNP (11a) and Neu4, 5Ac₂α2-3 GalβpNP (13a) were poorer substrates than Neu5Acα2-3GalβpNP (8a) but were both selectively cleaved by all influenza NAs tested but not human NEU2 or bacterial sialidases. The selectivity for influenza NAs was observed under both assay conditions but was more pronounced with the high-enzyme-concentration assay conditions which used 10-fold higher enzyme concentrations (except for Vc sialidase which was used at a 4-fold higher concentration) and a reaction time of 1 hour. Unlike Neu4,5Ac₂α2-3GalβpNP (13a) which was a suitable substrate for influenza NAs but not human or bacterial sialidases, its more stable 4-N-acetyl derivative 4NAcNeu5Acα2-3GalβpNP (12a) was resistant to cleavage by all of the sialidases tested.

Despite the different preferences for α2-6-versus α2-3-sialyl linkages of sialic acid-receptors by hemagglutinins (HAs) of IAV isolates from human versus avian and equine species, respectively, NAs from different IAV and IBV strains are more active towards α2-3-linked sialoside substrates. The selective cleavage of 4N₃Neu5Acα2-6GalβpNP (11b) by influenza NAs tested were observed only under the high-enzyme-concentration assay conditions (1 hour reaction time) and were much less pronounced compared to its α2-3-linked counterpart 4N₃Neu5Acα2-3GalβpNP (11a). α2-6-sialylgalactosides containing 4N₃Neu5Ac were determined to be resistant to hydrolysis by an IAV NA.

4NH₂Neu5Ac- and 4-Guanidino-Neu5Ac-Sialosides Are Selective Inhibitors Against Influenza NAs. Similar to Neu5Ac2en, both 4NH₂Neu5Ac2en (1) and 4-guanidino-Neu5 Ac2en (or Zanamivir, 2) bind to the substrate-binding site of influenza NAs and are much stronger inhibitors. The observation that both 4NH₂Neu5Acα2-3/6GalβpNP (9a/b) and 4-guanidino-Neu5Acα2-3/6GalβpNP (10a/b) were very poor substrates for the influenza NAs tested under even high-enzyme-concentration assay conditions (FIG. 4, panel (b)) indicated that these sialosides could be effective inhibitors. In this a fluorescence-based inhibition assay platform, a 4-methylumbelliferyl (MU)-tagged sialoside Neu5Acα2-3GalβMU (18) (8.0 μM) was used as a sialidase substrate in a 96-well plate. In the absence of an inhibitor, a sialidase can catalyze the cleavage of Neu5 Ac to form GalβMU, which is then cleaved by the excess amount of β-galactosidase in the reaction mixture to release the fluorescent molecule MU that can be detected by a plate reader.

Indeed, when Neu5Acα2-3GalβMU (8.0 μM) was used as the substrate and the same concentration of a pNP-tagged sialoside was used as an inhibitor, 4-guanidino-Neu5 Acα2-3 GalβpNP (10a) and 4-guanidino-Neu5Acα2-6GalβpNP (10b) showed 80-98% and 67-93%, inhibitory activities, respectively, against the influenza NAs tested (Table 4). Inhibitory activity of 4NH₂Neu5Acα2-3GalβpNP (9a) was also significant against N₁-Vic19 (48.3±7.9%) and N1-BR18 (47.2±3.3%). In contrast, no significant inhibitory activity of the pNP-tagged sialosides against human cytosolic sialidase hNEU2, bacterial Streptococcus pneumoniae sialidase SpNanA, and the sialidase activity of a multifunctional Pasteurella multocida α2-3-sialyltransferase 1 (PmST1) in the presence of cytidine-5′-monophosphate (CMP) (Table 5) was observed under the same assay conditions. The inhibitory activity of 4NH₂Neu5Acα2-6GalβpNP (9b) was insignificant against the N1 or N2 NAs tested and was weaker against NB-NB-Aus21 and NB-Phu13 compared to 4NH₂Neu5Acα2-3GalβpNP (9a).

Neu5 Ac2en (2) displayed inhibitory activity against all sialidases tested except for the sialidase activity of PmST1 (Table 4 and Table 5). Its inhibitory activity against N₂s and hNEU2 was generally lower than other sialidases tested. In comparison, 4NH₂Neu5Ac2en (3) and Zanamivir (4) were selective inhibitors against influenza NAs. Selective inhibitory activity of 4N₃Neu5Ac2en (5) was observed for the IBV NAs NB-NB-Aus21 (78.8±3.7%) and NB-Phu13 (75.6±1.6%). 4NAcNeu5Ac2en (6) was not an effective inhibitor against any of the sialidases tested.

TABLE 4
shows percentage inhibition (%) of pNP-tagged sialosides containing a
4-N-modified Neu5Ac and Sia2en compounds (8.0 μM) against IAV NAs
(N1-Vic19, N1-BR18, N2-Dar21, N2-Kan17) and IBV NAs (NB-NB-Aus21, NB-
Phu13). Percentage inhibitions higher than 47% are highlighted in bold.

| | NA | | | | | |
|---|---|---|---|---|---|---|
| | N1- | N1- | N2- | N2- | NB-NB- | NB- |
| Inhibitor | Vic19 | BR18 | Dar21 | Kan17 | Aus21 | Phu13 |
| Neu5Acα2-3GalβpNP (8a) | 3.3 ± | 1.8 ± | 0.9 ± | 0.1 ± | 0.5 ± | 0.7 ± |
| | 5.6 | 1.4 | 2.6 | 4.2 | 2.4 | 1.3 |
| Neu5Acα2-6GalβpNP (8b) | 1.4 ± | 3.5 ± | 0.8 ± | 0.4 ± | 0.2 ± | 0.8 ± |
| | 2.7 | 1.5 | 2.3 | 6.5 | 2.2 | 9.5 |
| 4NH₂Neu5Acα2-3GalβpNP (9a) | 48.3 ± | 47.2 ± | 13.4 ± | 0.5 ± | 28.5 ± | 18.3 ± |
| | 7.9 | 3.3 | 7.3 | 7.2 | 1.6 | 0.6 |
| 4NH₂Neu5Acα2-6GalβpNP (9b) | 0.4 ± | 0.7 ± | 2.9 ± | 1.5 ± | 13.5 ± | 9.8 ± |
| | 3.8 | 2.9 | 2.1 | 9.2 | 4.4 | 2.2 |
| 4-Guanidino-Neu5Acα2- | 98.2 ± | 97.9 ± | 97.0 ± | 94.9 ± | 84.2 ± | 80.1 ± |
| 3GalβpNP (10a) | 0.0 | 0.0 | 0.0 | 0.8 | 0.8 | 1.5 |
| 4-Guanidino-Neu5Acα2- | 93.1 ± | 92.8 ± | 81.5 ± | 78.0 ± | 66.6 ± | 78.9 ± |
| 6GalβpNP (10b) | 0.4 | 0.8 | 1.0 | 1.1 | 1.8 | 0.8 |
| 4N₃Neu5Acα2-3GalβpNP (11a) | 1.2 ± | 2.6 ± | 0.8 ± | 0.1 ± | 3.8 ± | 0.9 ± |
| | 7.7 | 4.7 | 4.1 | 1.6 | 5.9 | 3.3 |
| 4N₃Neu5Acα2-6GalβpNP (11b) | 3.9 ± | 0.2 ± | 1.0 ± | 0.0 ± | 1.4 ± | 2.6 ± |
| | 1.9 | 5.1 | 8.7 | 2.4 | 2.5 | 1.2 |
| 4NAcNeu5Acα2-3GalβpNP | 1.5 ± | 3.0 ± | 3.2 ± | 2.1 ± | 0.1 ± | 0.9 ± |
| (12a) | 17.2 | 2.5 | 5.9 | 1.5 | 1.2 | 5.1 |
| 4NAcNeu5Acα2-6GalβpNP | 0.4 ± | 0.0 ± | 0.4 ± | 1.0 ± | 0.4 ± | 1.5 ± |
| (12b) | 4.2 | 1.9 | 4.4 | 1.3 | 1.2 | 8.1 |
| Neu4,5Ac₂α2-3GalβpNP (13a) | 1.5 ± | 2.0 ± | 7.6 ± | 1.6 ± | 0.8 ± | 1.3 ± |
| | 5.8 | 0.3 | 5.3 | 0.7 | 5.6 | 2.3 |
| Neu5Ac2en (2) | 76.2 ± | 74.3 ± | 49.9 ± | 39.6 ± | 78.7 ± | 76.2 ± |
| | 2.4 | 2.5 | 1.7 | 7.3 | 1.4 | 1.3 |
| 4NH₂Neu5Ac2en (3) | 95.9 ± | 94.9 ± | 94.6 ± | 91.3 ± | 97.4 ± | 91.2 ± |
| | 0.3 | 1.1 | 0.8 | 3.6 | 0.1 | 0.3 |
| Zanamivir (4) | 99.8 ± | 99.6 ± | 99.7 ± | 95.6 ± | 99.1 ± | 95.3 ± |
| | 0.0 | 0.0 | 0.2 | 0.8 | 0.4 | 0.5 |
| 4N₃Neu5Ac2en (5) | 39.6 ± | 39.2 ± | 22.1 ± | 18.3 ± | 78.8 ± | 75.6 ± |
| | 5.0 | 7.9 | 3.3 | 8.4 | 3.7 | 1.6 |
| 4NAcNeu5Ac2en (6) | 0.7 ± | 2.5 ± | 2.4 ± | 6.1 ± | 1.4 ± | 0.6 ± |
| | 2.3 | 5.4 | 1.5 | 5.2 | 4.1 | 1.4 |

TABLE 5
shows percent inhibition of pNP-tagged sialosides containing a 4-N-modified
Neu5Ac and Sia2en derivatives (8.0 μM) against PmST1, SpNanA, and
hNEU2. Inhibition levels higher than 47% are highlighted in bold.

| | NA | | |
|---|---|---|---|
| Inhibitor | PmST1 WT | SpNanA | hNEU2 |
| Neu5Acα2-3GalβpNP (8a) | 12.4 ± 2.2 | 17.1 ± 6.1 | 0.0 ± 5.0 |
| Neu5Acα2-6GalβpNP (8b) | 14.6 ± 6.4 | 5.8 ± 6.8 | 0.0 ± 6.7 |
| 4NH₂Neu5Acα2-3GalβpNP (9a) | 23.4 ± 1.2 | 9.0 ± 8.3 | 0.0 ± 2.0 |
| 4NH₂Neu5Acα2-6GalβpNP (9b) | 35.7 ± 19.9 | 4.4 ± 9.6 | 0.0 ± 2.5 |
| 4-Guanidino-Neu5Acα2-3GalβpNP (10a) | 26.1 ± 0.1 | 0.2 ± 0.7 | 0.0 ± 0.6 |
| 4-Guanidino-Neu5Acα2-6GalβpNP (10b) | 31.7 ± 13.7 | 6.4 ± 0.3 | 0.0 ± 3.2 |
| 4N₃Neu5Acα2-3GalβpNP (11a) | 24.0 ± 6.9 | 0.6 ± 2.7 | 0.0 ± 1.9 |
| 4N₃Neu5Acα2-6GalβpNP (11b) | 41.3 ± 20.3 | 11.6 ± 6.7 | 0.0 ± 3.6 |
| 4NAcNeu5Acα2-3GalβpNP (12a) | 27.9 ± 4.2 | 10.0 ± 2.3 | 0.0 ± 2.3 |
| 4NAcNeu5Acα2-6GalβpNP (12b) | 18.6 ± 4.0 | 4.2 ± 0.8 | 0.0 ± 1.7 |
| Neu4,5Ac₂α2-3GalβpNP | 38.7 ± 1.6 | 2.8 ± 5.4 | 0.0 ± 7.0 |
| Neu5Ac2en (2) | 0.9 ± 8.9 | 68.3 ± 2.7 | 37.1 ± 1.1 |
| 4NH₂Neu5Ac2en (3) | 9.8 ± 1.2 | 7.9 ± 2.6 | 0.0 ± 6.6 |
| Zanamivir (4) | 17.4 ± 9.3 | 0.5 ± 7.3 | 26.0 ± 3.6 |
| 4N₃Neu5Ac2en (5) | 15.4 ± 5.0 | 3.0 ± 1.3 | 3.3 ± 0.6 |
| 4NAcNeu5Ac2en (6) | 7.1 ± 0.7 | 0.5 ± 6.5 | 2.1 ± 2.5 |

IC₅₀ values (μM) were determined for the compounds with significant inhibitory activities against IAV and IBV NAs in the inhibition assays. As shown in Table 6 below, Neu5Ac2en was a micromolar inhibitor whereas Zanamivir (4) was a nanomolar inhibitor against influenza NAs which is consistent with previous reports. The inhibition efficiency of sialoside 4-guanidino-Neu5Acα2-3 GalβpNP (10a) was better than 4NH₂Neu5Ac2en (3) against N1-Vic19 and N₁-BR18 (about 4-fold better), as well as N2-Kan17 and N2-Dar21 (about 10-fold better), although it was still ~10-fold less efficient than Zanamivir (4) in inhibiting these IAV NAs. 4-Guanidino-Neu5Acα2-6GalβpNP (10b) with an α2-6-sialyl linkage was generally a weaker NA inhibitor than its α2-3-sialyl linked counterpart 4-guanidino-Neu5 Acα2-3 GalβpNP (10a), agreeing well with the data shown in Table 4. Therefore, α2-3-linked 4-guanidino-Neu5Ac-sialosides can be considered as a class of sialidase inhibitors that are based on substrate-analogs. Compared to sialidase transition state analog Neu5 Ac2en-derived inhibitors, these sialidase substrate-analog-based inhibitors better resemble the native NA substrates and thus are less prone to suffer from the development of resistant strains compared to the clinically used oseltamivir as demonstrated for the mechanism-based selective anti-influenza virus inhibitors which are α-sialyl fluorides containing 3-fluoro and 4-amino- or 4-guanodino-Neu5 Ac. In addition, the sialidase substrate-analog-based inhibitors have the advantage of allowing immobilization or conjugation of the inhibitor via a linker that can be easily installed at its reducing end as demonstrated below.

TABLE 6
shows IC₅₀ values (μM) of 4-guanidino-sialosides
(10a/b), Neu5Ac2en (2) and its 4-N-derivatives (3-5).

| | NA | | | | | |
|---|---|---|---|---|---|---|
| Inhibitor | N1-Vic19 | N1-BR18 | N2-Dar21 | N2-Kan17 | NB-NB-Aus21 | NB-Phu13 |
| 4-Guanidino- | (3.4 ± 0.2) × | (4.3 ± 0.4) × | (2.3 ± 0.2) × | (1.9 ± 0.2) × | 1.2 ± 0.1 | 1.4 ± 0.1 |
| Neu5Acα2- | 10⁻² | 10⁻² | 10⁻¹ | 10⁻¹ | | |
| 3GalβpNP (10a) | | | | | | |
| 4-Guanidino- | (3.1 ± 0.3) × | (3.5 ± 0.3) × | 2.0 ± 0.2 | 1.7 ± 0.1 | 3.2 ± 0.3 | (8.3 ± 1.0) × |
| Neu5Acα2- | 10⁻¹ | 10⁻¹ | | | | 10⁻¹ |
| 6GalβpNP (10b) | | | | | | |
| Neu5Ac2en (2) | 1.1 ± 0.1 | 1.9 ± 0.1 | 6.8 ± 0.7 | 8.4 ± 0.6 | 1.5 ± 0.2 | 1.6 ± 0.1 |
| 4NH₂Neu5Ac2en | (1.3 ± 0.1) × | (1.8 ± 0.2) × | (2.1 ± 0.1) × | (2.0 ± 0.1) × | (9.1 ± 0.7) × | (1.3 ± 0.1) × |
| (3) | 10⁻¹ | 10⁻¹ | 10⁻¹ | 10⁻¹ | 10⁻² | 10⁻¹ |
| Zanamivir (4) | (3.4 ± 0.2) × | (2.3 ± 0.2) × | (9.0 ± 0.9) × | (1.1 ± 0.1) × | (1.0 ± 0.1) × | (1.1 ± 0.1) × |
| | 10⁻³ | 10⁻³ | 10⁻³ | 10⁻² | 10⁻² | 10⁻² |
| 4N₃Neu5Ac2en | 6.2 ± 0.4 | 4.4 ± 0.2 | (1.7 ± 0.3) × | (2.6 ± .7) × | 1.5 ± 0.1 | 1.4 ± 0.1 |
| (5) | | | 10¹ | 10¹ | | |

### Chemoenzymatic Synthesis of Biotinylated Sialosides as Affinity Ligands for Influenza

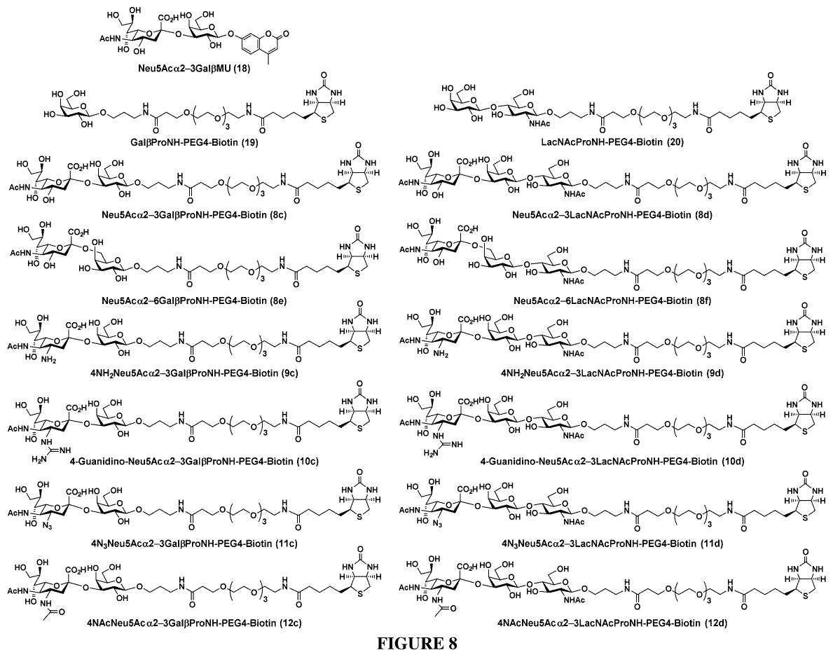

NAs. The strong inhibitory activity of 4-guanidino-Neu5Acα2-3GalβpNP (10a) against the NAs from IAVs (N1 and N₂) indicated that this compound could be used as an affinity ligand to selectively purify NAs and IAVs for vaccine development. Biotinylated polyethylene oligomer (PEG4)-linked galactosides GalβProNH-PEG4-Biotin (19) and LacNAcβProNH-PEG4-Biotin (20) were synthesized (ESI) and used as sialyltransferase acceptors and negative controls for binding studies. α2-3-Linked sialosides 4NH₂Neu5Acα2-3GalβpProNH-PEG4-Biotin (9c) (FIG. 8) and 4NH₂Neu5Acα2-3LacNAcβpProNH-PEG4-Biotin (9d) were chemoenzymatically synthesized from 4NH₂Neu5Ac (7) and the corresponding biotinylated galactoside (19) and biotinylated LacNAc-glycoside (20), respectively, using the OP2E sialylation strategy similar to that described above and illustrated in Scheme 1 (b). They were chemically derivatized at the 4-amino group of the 4NH₂Neu5 Ac residue to form sialosides containing 4-guanidino-Neu5Ac (10c and 10d) (FIG. 8), 4N₃Neu5Ac (11c and 11d), and 4NAcNeu5Ac (12c and 12d), respectively. The corresponding Neu5 Acα2-3Gal/LacNAcβProNH-PEG4-Biotin (8c/d) and Neu5 Acα2-6Gal/LacNAcβProNH-PEG4-Biotin (8e/f) were synthesized from Neu5Ac using a similar OP2E sialylation strategy and used as controls.

Enzyme-linked immunosorbent assays (ELISA) using neutravidin-coated 384-well plates showed (FIG. 5, panel (a)) that both 4NH₂Neu5Acα2-3Gal/LacNAcβProNH-PEG4-Biotin (9c/d) and 4-guanidino-Neu5Acα2-3Gal/LacNAcβProNH-PEG4-Biotin (10c/d) were indeed suitable ligands for immobilizing the NAs from both IAVs and IBVs as detected by a horseradish peroxidase (HRP)-conjugated anti-histidine/strep antibody followed by adding an HRP substrate 3,3′,5,5′-tetramethylbenzidine (TMB). In comparison, galactoside (19) and LacNAc-glycoside (20) and the corresponding Neu5 Ac-sialosides (8c-8f), 4N₃Neu5Ac-sialosides (11c/d), and 4NAcNeu5Ac-sialosides (12c/d) were not suitable affinity ligands. The lack of the binding can be due to the absence of or the weak interactions between the NAs and the glycosides (e.g. galactoside 19, LacNAc-glycoside 20, and 4NAcNeu5 Ac-sialosides 12c/d), or cleavage of the terminal sialic acids (e.g., Neu5 Ac-sialosides 8c-f, 4N₃Neu5Ac-sialosides 11c/d). Noteworthy, 4NH₂Neu5Ac-sialosides (9c/d) showed weaker binding to N2-Dar21 and N2-Kan17, compared to other influenza NAs, suggesting NAs from these H3N2 IAVs generally have lower affinity for 4NH₂Neu5 Ac-sialosides (9c/d).

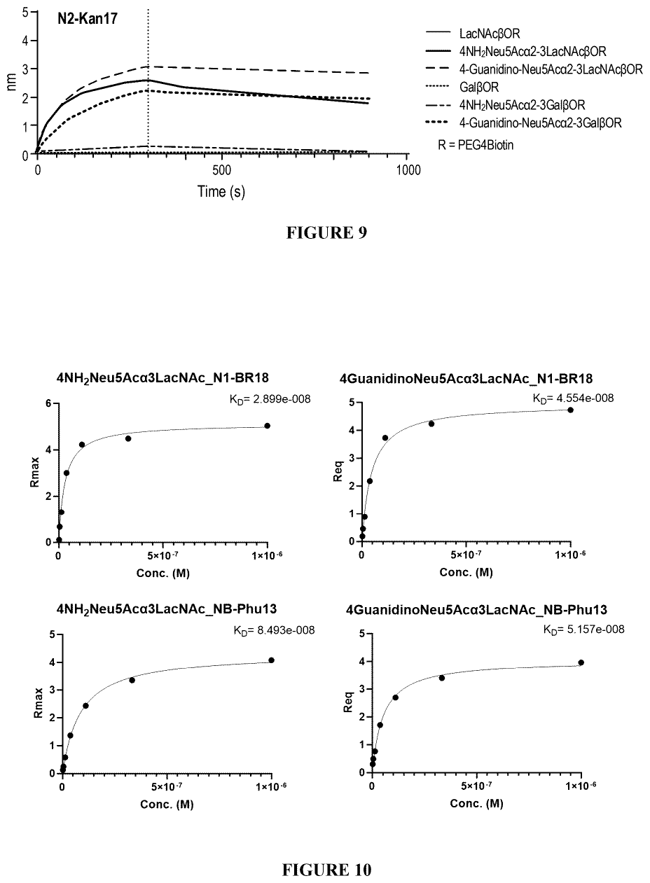

Bio-layer interferometry (BLI) experiments were carried out to investigate the binding affinity between biotinylated sialosides immobilized on streptavidin-coated biosensors and influenza NAs as analytes. Real-time BLI assays for N2-Kan17 with sialyl LacNAc-glycosides and sialyl Gal-glycosides containing 4NH₂- or 4-guanidino-derivative of Neu5Ac (FIG. 9) showed that sialyl LacNAc-glycosides were better binders than their sialyl Gal-glycoside counterparts, aligning well with the ELISA results (FIG. 5, panel (a)). Therefore, the α2-3-linked sialyl LacNAc-glycosides were chosen for more detailed BLI assays. As shown in FIG. 5, panel (b), 4-guanidino-(10d) and 4NH₂-(9d) derivatives are significantly stronger binders than other sialosides. Weak binding of N1-Vic19, N2-Dar21, and the IBV NAs to the 4N₃-derivative (11d) and weak binding of N1-Vic19 and N2-Dar21 to the 4NAc-derviative (12d) were also observed.

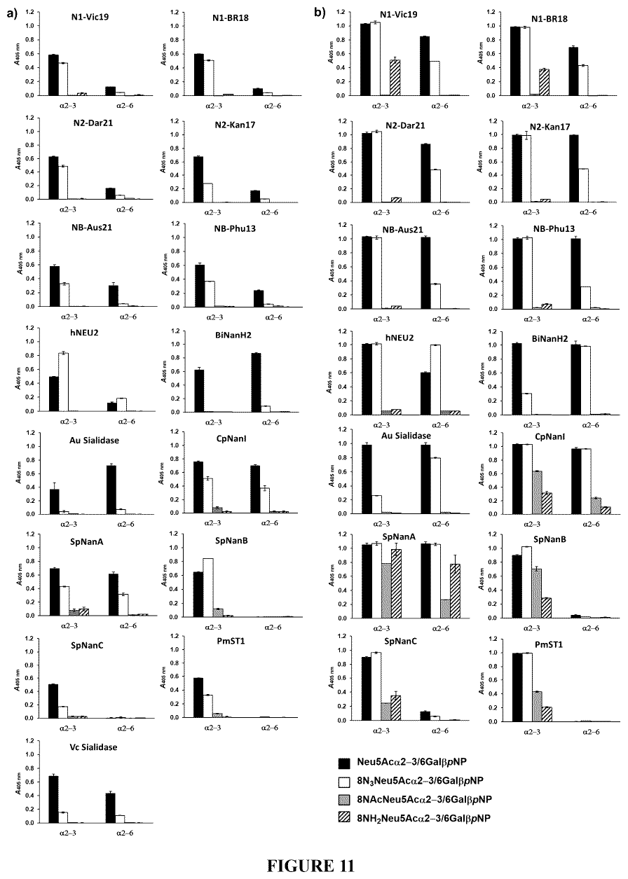

To obtain binding kinetic data of 4NH₂- or 4-guanidino-Neu5Acα2-3LacNAcβProNH-PEG4-Biotin (9d or 10d), influenza NAs in serial dilutions were used as analytes in a steady-state analysis (FIG. 5, panel (c)). The results showed that 4NH₂-modified sialoside (9d) bound to the NAs but was partially cleaved off during the incubation. In comparison, the 4-guanidino-modified sialoside (10d) was resistant to the NA-catalyzed cleavage. By fitting equilibrium response curves during the association phase and the NA concentration to the dissociation rate constant equation (FIG. 10), the K_{D} values (2.90-8.49×10⁻⁸ M) were in the nanomolar range. The K_{D} value (4.55×10⁻⁸ M) obtained for the interaction of N1-BR18 to 4-guanidino-Neu5Acα2-3LacNAcβProNH-PEG4-Biotin (10d) was comparable to the IC₅₀ value, (4.3±0.4)×10⁻⁸ M, determined by the inhibition studies for the counterpart 4-guanidino-Neu5Acα2-3GalβpNP (10a) (Table 3).

FIG. 5 shows in panel (a) 4NH₂Neu5Acα2-3Gal/LacNAcβpProNH-PEG4-Biotin (9c/d) and 4-guanidino-Neu5Acα2-3Gal/LacNAcβpProNH-PEG4-Biotin (10c/d) are suitable affinity ligands for recombinant NAs from both IAVs and IBVs as demonstrated by ELISA. Black bars, GalβProNH-PEG4-Biotin (19) and the corresponding α2-3-linked (by default, not labeled on the x-axis) or α2-6-linked (Neu5Ac only, labeled on the x-axis) sialosides; gray bars, LacNAcβProNH-PEG4-Biotin (20), and the corresponding sialosides. FIG. 5, panel (b) shows BLI assay results for IAV and IBV NAs binding to sialyl LacNAc-glycosides. FIG. 5, panel (c) shows Kp values determined by real-time BLI assays for N1-BR18 and NB-Phul34 with NH₂Neu5Acα2-3LacNAcβProNH-PEG4-Biotin (9d) or 4-guanidino-Neu5Acα2-3LacNAcβProNH-PEG4-Biotin (10d).

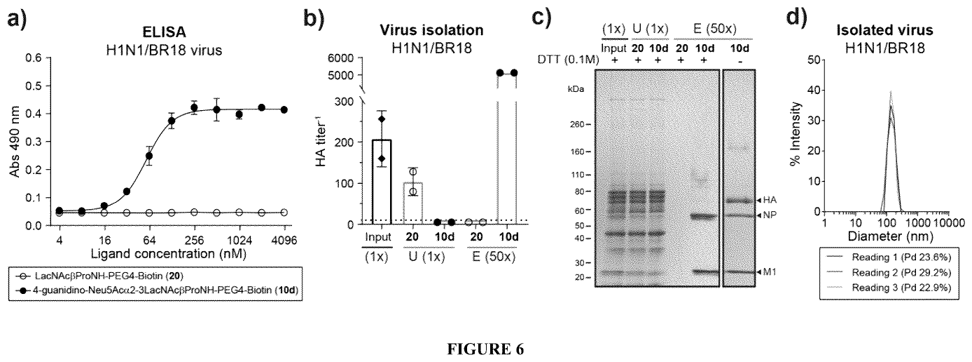

FIG. 6 shows 4-Guanidino-Neu5Acα2-3LacNAcβProNH-PEG4-Biotin (10d) is an effective affinity ligand for detecting and purifying H1N1 virus as demonstrated by (a) ELISA using streptavidin-coated 96-well plates and (b-d) streptavidin-Dynabeads with LacNAcβProNH-PEG4-Biotin (20) as a negative control ligand. FIG. 6, panel (a) shows binding of H1N1/BR18 virus in egg allantoic fluid (input) to streptavidin-coated wells incubated with the indicated concentration of 10d or 20 was detected with a monoclonal HA antibody. Data are the mean±SD of an assay run in duplicate; FIG. 6, panel (b) shows isolation of H1N1/BR18 virus by streptavidin-Dynabeads containing 10d or 20 was monitored by measuring HA titers in the input, unbound (U), and elution (E) fractions. FIG. 6, panel (c) shows purity of the isolated H1N1/BR18 virus was analyzed by SDS-PAGE and Coomassie staining of the input, U and E fractions obtained with 10d and the negative control 20. E fraction from 10d was resolved without dithiothreitol (DTT) and bands corresponding to the viral hemagglutinin (HA), nucleoprotein (NP) and matrix 1 (M1) proteins are shown. FIG. 6, panel (d) shows diameter and polydispersity (Pd) of the H1N1/BR18 virus isolated with 10d was measured by dynamic light scattering.

Biotinylated 4-Guanidino-Sialosides as Affinity Ligands for Purifying Influenza viruses. The experiments discussed herein above indicated that the immobilized 4-guanidino-Neu5Acα2-3LacNAcβProNH-PEG4-Biotin (10d) and related 4-guanidino-Neu5Ac-sialosides could be used as affinity ligands to isolate influenza viruses for downstream applications. Specific binding of the vaccine strain A/Brisbane/02/2018 (H1N1) in egg allantoic fluid by ELISA using streptavidin-coated 96-well plates (FIG. 6, panel (a)) was observed. In addition, 4-guanidino-Neu5Acα2-3LacNAcβProNH-PEG4-Biotin (10d)-immobilized streptavidin beads were efficient in retaining H1N1/BR18 virions (FIG. 6, panel (b)) which were readily eluted using 50 mM of Neu5 Ac2en. In comparison, streptavidin beads immobilized with the control glycan LacNAcβProNH-PEG4-Biotin (20) did not retain influenza virions. SDS-PAGE analysis (FIG. 6, panel (c)) indicated that the elution fraction contained the expected viral protein bands with almost no contaminating allantoic fluid proteins. Dynamic light scattering analysis showed that eluted virus was homogeneous with reasonable polydispersity (Pd<30%) and a mean diameter of ~125 nm (FIG. 6, panel (d)), confirming that the approach had indeed isolated virus efficiently.

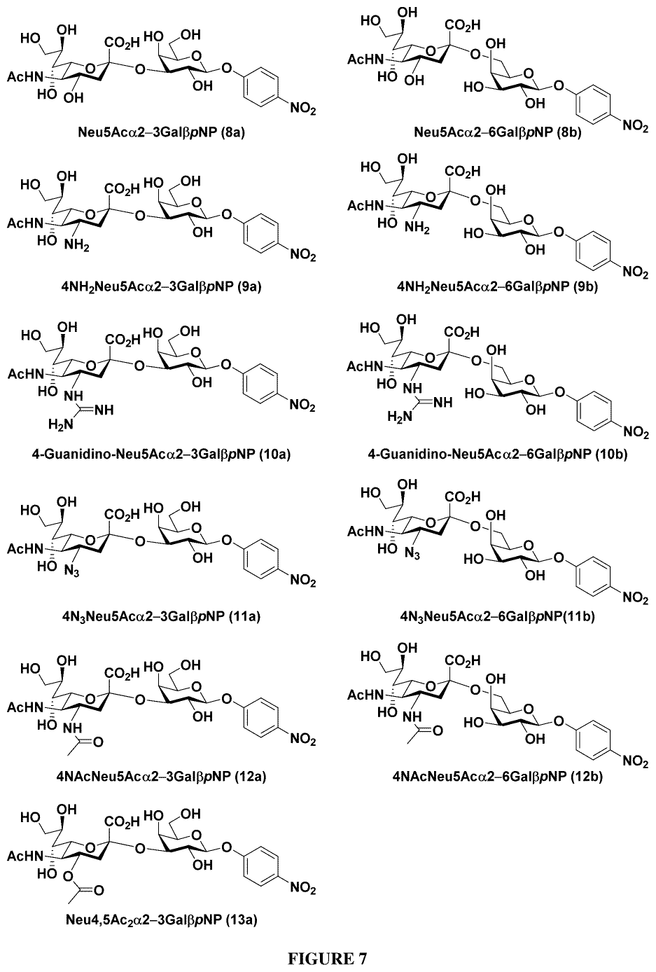

Synthesis of 4-N-Sialosides. FIG. 7 shows structures of sialosides synthesized and used for sialidase substrate specificity and inhibition assays. FIG. 8 shows structures of Neu5Acα2-3GalβMU (18) and biotinylated glycans (19-20, 8c-f, 9c-d, 10c-d, 11c-d, and 12c-d). FIG. 9 shows Bio-Layer Interferometry (BLI) assays using IAV NA N2-Kan17 showed better binding of sialylated LacNAc-glycosides compared to sialylated Gal-glycosides. FIG. 10 shows steady state analysis of sialoside binding to (a) N1-BR18 and (b) NB-Phu13 with NH₂Neu5Acα2-3LacNAcβProNH-PEG4-Biotin (9d) or 4-guanidino-Neu5Acα2-3LacNAcβProNH-PEG4-Biotin (10d) by real-time BLI assays.

Materials and general methods. Chemicals were purchased and used without further purification. GalβpNP and GalβMU were from Fisher Scientific. Nuclear magnetic resonance (NMR) spectra were recorded in the NMR facility of the University of California, Davis on 600 MHz and 800 MHz Bruker Avance III-NMR spectrometers. Chemical shifts are reported in parts per million (ppm) on the 8 scale. High resolution electrospray ionization (ESI) mass spectra were obtained using a Thermo Electron LTQ-Orbitrap Hybrid mass spectrometer at the mass spectrometry facility at the University of California, Davis. Column chromatography was performed using a CombiFlash® Rf 200i system with an ODS-SM (C18) column (14 g, 50 μm, 120 Å, Yamazen) or manually using columns packed with silica gel 60 Å (230-400 mesh, Sorbent Technologies). Gel filtration chromatography was performed with a column (100 cm×2.5 cm) packed with Bio-Gel P-2 Fine resins (Bio-Rad). Thin layer chromatography (TLC) was performed on silica gel plates (Sorbent Technologies) using anisaldehyde sugar stain or 5% sulfuric acid in ethanol stain for detection. Arthrobacter ureafaciens sialidase was purchased from EY laboratories, Inc. (San Mateo, CA, USA). Vibrio cholerae sialidase, Clostridium perfringens sialidase (CpNanI), and β-galactosidase from Aspergillus oryzae (≥8 units/mg solid) were purchased from Sigma-Aldrich. Recombinant Neisseria meningitidis CMP-sialic acid synthetase (NmCSS), Pasteurella multocida α2-3-sialyltransferase 1 (PmST1) and its M144D mutant (PmST1 M144D), Campylobacter jejuni Cst-I (CjCst-I), Photobacterium sp. α2-6-sialyltransferase mutant (Psp2,6ST_A366G), human cytoplasmic sialidase hNEU2, Streptococcus pneumoniae sialidases SpNanA, SpNanB, and SpNanC, and Bifidobacterium infantis sialidase (BiNanH2) were expressed and purified as described in Yu, H. et al., Bioorg. Med. Chem. 2004, 12, 6427-6435; Yu, H. et al. J. Am. Chem. Soc. 2005, 127, 17618-17619; Sugiarto, G. et al., ACS Chem. Biol. 2012, 7, 1232-1240; Zhang, L. et al., Molecules 2023, 28, 2753; Li, Y. et al., Mol. Biosyst. 2011, 7, 1060-1072, Tasnima, N. et al., Org. Biomol. Chem. 2016, 15, 160-167; Xiao, A. et al., J. Org. Chem. 2018, 83, 10798-10804; Xiao, A. et al., ACS Catal. 2018, 8, 43-47; and Sela, D. A. et al., J. Biol. Chem. 2011, 286, 11909-11918, which are incorporated herein by reference.

IAV and IBV NAs were expressed in Sf9 cells using baculovirus expression systems. His₆-tagged tetrabrachion tetramerization domain-containing N1-BR18 (82-469 aa) and N2-Kan17 (74-469 aa) were from the respective IAV strains A/Brisbane/02/2018 (H1N1) or A/Kansas/14/2017 (H3N2); Strep-tagged tetrabrachion tetramerization domain-containing N1-Vic19 (35-469 aa) and N2-Dar21 (74-469 aa) were from the respective strains A/Victoria/2570/2019 (H1N1) and A/Darwin/9/2021 (H3N2); Strep-tagged tetrabrachion tetramerization domain-containing NB-NB-Aus21 (39-466 aa) and NB-Phu13 (39-466 aa) were from the respective strains B/Austria/1359417/2021 and B/Phuket/3073/2013. Pierce™ streptavidin and NeutrAvidin-coated plates were purchased from Thermo Fisher Scientific. The binding was detected using a horseradish peroxidase (HRP)-conjugated anti-His₆ monoclonal antibody (ThermoFisher Scientific) or HRP-conjugated anti-StrepTag II monoclonal antibody (Sigma-Aldrich), and a colorimetric substrate solution 3,3′,5,5′-tetramethylbenzidine (TMB) (ThermoFisher Scientific). Biolayer interferometry (BLI) assays were performed using a Octet® RED384 instrument in the Octet Real-Time Drug and Protein Binding Kinetics Unit at the University of California Davis. Octet® high precision streptavidin (SAX) Biosensors, Octet® kinetics buffer 10×, and Octet® 384-well titted-bottom plates were purchased from Sartorius Corporation. Dynabeads MyOne Streptaidin T1 were from Invitrogen. No unexpected or unusually high safety hazards were encountered.

Synthesis of Neu5Acα2-3GalβMU (18). GalβMU (100 mg, 0.30 mmol), CTP (234 mg, 0.44 mmol), and Neu5Ac (119 mg, 0.38 mmol) were dissolved in water in a 50 mL centrifuge tube containing Tris-HCl buffer (100 mM, pH 8.5) and MgCl₂ (20 mM). NmCSS (3 mg) and CjCst-I (2 mg) were then added. The reaction mixture (20 mL) was incubated at 30° C. with agitation at 180 rpm. The product formation was monitored by high-resolution mass spectrometry (HRMS). After 16 hours (h), the reaction mixture was incubated in a boiling water bath for 5 minutes (min) to denature the enzymes, cooled down to room temperature, then centrifuged at 8000 rpm for 30 min at 4° C. The supernatant was concentrated and purified by a preconditioned DSC-18 SPE cartridge (bed wt. 10 g). After washing the cartridge with water (30 mL), a mixture solvent of methanol in water (40%) was used to elute the product. The solvent was removed by evaporation in vacuo and the pure product was obtained as a white powder (171 mg, 89% yield) and characterized by nuclear magnetic resonance (NMR) spectroscopy. ¹H NMR (800 MHz, D₂O) δ 7.45 (d, J=8.9 Hz, 1H), 6.97 (dd, J=8.8, 2.5 Hz, 1H), 6.84 (d, J=2.5 Hz, 1H), 5.98 (t, J=1.2 Hz, 1H), 5.15 (d, J=7.8 Hz, 1H), 4.27 (dd, J=9.8, 3.3 Hz, 1H), 4.08 (d, J=3.6 Hz, 1H), 3.95-3.86 (m, 4H), 3.85-3.77 (m, 3H), 3.74-3.68 (m, 2H), 3.67 (dd, J=10.4, 2.1 Hz, 1H), 3.65-3.60 (m, 2H), 3.17 (q, J=7.4 Hz, 1H), 2.81 (dd, J=12.5, 4.8 Hz, 1H), 2.23 (d, J=1.1 Hz, 3H), 2.03 (s, 3H), 1.86 (t, J=12.2 Hz, 1H), 1.25 (t, J=7.4 Hz, 2H). ¹³C NMR (200 MHz, D₂O) δ 174.98, 173.84, 164.03, 159.39, 155.83, 153.43, 126.42, 114.66, 113.93, 110.94, 103.27, 99.95, 99.84, 75.51, 75.26, 72.90, 71.74, 68.78, 68.37, 68.01, 67.24, 62.46, 60.68, 59.30, 51.69, 46.63, 39.73, 22.04, 17.86, 8.21. HRMS (ESI-Orbitrap) m/z: [M−H]⁻ calculated for C₂₇H₃₄NO₁₆ 628.1883; found 628.1884.

General procedures for synthesizing biotinylated glycan acceptors 19 and 20 GalβProN₃ or LacNAcβProN₃ (100-150 mg) was dissolved in water-methanol solution (12 mL, 1:2 by volume), a catalytic amount of 10% palladium on charcoal was added to a 100 mL round bottom flask. The mixture was stirred under a hydrogen environment for 2 hours. The solution was passed through a syringe filter and concentrated. After lyophilization, the obtained glycosyl propylamine was dissolved in anhydrous DMF (10 mL) and was added to a solution of biotin-PEG4—CO₂H (1.05 equiv.), HBTU (1.1 equiv.), and DIPEA (3.5 equiv.) in anhydrous DMF (5 mL). The reaction mixture was stirred at room temperature for 24 hours. The solvent was removed by evaporation in vacuo and the residue was purified by Bio-gel P2 to obtain the desired pure product.

GalβProNH-PEG4-Biotin (19). 261 mg, 87% yield. ¹H NMR (600 MHz, D₂O) δ 4.62 (dd, J=7.8, 4.8 Hz, 1H), 4.43 (dd, J=7.8, 4.2 Hz, 1H), 4.39 (d, J=7.8 Hz, 1H), 3.97 (dt, J=10.2, 6.0 Hz, 1H), 3.93 (d, J=3.6 Hz, 1H), 3.86-3.58 (m, 23H), 3.52 (dd, J=9.6, 7.8 Hz, 1H), 3.40 (t, J=5.4 Hz, 2H), 3.36-3.28 (m, 3H), 3.01 (dd, J=13.2, 5.4 Hz, 1H), 2.79 (d, J=13.2 Hz, 1H), 2.52 (t, J=6.0 Hz, 2H), 2.29 (t, J=7.2 Hz, 2H), 1.85 (p, J=6.6 Hz, 2H), 1.79-1.53 (m, 4H), 1.48-1.38 (m, 2H). ¹³C NMR (150 MHz, D₂O) δ 176.87, 173.88, 165.29, 102.82, 75.13, 72.77, 70.77, 69.65, 69.62, 69.60, 69.56, 69.54, 69.50, 69.41, 69.33, 68.88, 68.63, 67.66, 67.62, 66.80, 62.07, 60.97, 60.24, 55.35, 39.69, 38.89, 36.31, 36.08, 35.46, 28.45, 27.89, 27.87, 27.70, 25.14. HRMS (ESI-Orbitrap) m/z: [M+Na]⁺ calculated for C₃₀H₅₄N₄NaO₁₃S 733.3306; found 733.3306.

LacNAcβProNH-PEG4-Biotin (20). 222 mg, 89% yield. ¹H NMR (800 MHz, D₂O) δ 4.60 (dd, J=8.0, 4.8 Hz, 1H), 4.50 (d, J=8.0 Hz, 1H), 4.46 (d, J=8.0 Hz, 1H), 4.41 (dd, J=8.0, 4.8 Hz, 1H), 3.97 (dd, J=12.8, 2.4 Hz, 1H), 3.94-3.88 (m, 2H), 3.82 (dd, J=12.8, 5.6 Hz, 1H), 3.80-3.55 (m, 23H), 3.53 (dd, J=9.6, 8.0 Hz, 1H), 3.38 (t, J=6.4 Hz, 2H), 3.35-3.30 (m, 1H), 3.27 (dt, J=14.4, 7.2 Hz, 1H), 3.18 (dt, J=13.6, 7.2 Hz, 1H), 2.98 (dd, J=12.8, 4.8 Hz, 1H), 2.77 (d, J=13.6 Hz, 1H), 2.51 (t, J=6.4 Hz, 2H), 2.27 (t, J=7.2 Hz, 2H), 2.03 (s, 3H), 1.85-1.53 (m, 6H), 1.50-1.36 (m, 2H). ¹³C NMR (200 MHz, D₂O) δ 176.88, 174.44, 173.83, 165.29, 102.86, 101.03, 78.46, 75.32, 74.74, 72.48, 72.40, 70.93, 69.62, 69.60, 69.58, 69.54, 69.48, 69.39, 68.85, 68.51, 67.69, 66.78, 62.05, 60.98, 60.22, 60.05, 55.33, 55.06, 39.67, 38.87, 36.22, 36.02, 35.43, 28.35, 27.86, 27.67, 25.11, 22.13. HRMS (ESI-Orbitrap) m/z: [M+H]⁺ calculated for C₃₈H₆₈N₅O₁₈S 914.4280; found 914.4279.

Methyl 5-acetamido-2,4,7,8,9-penta-O-acetyl-3,5-dideoxy-D-glycero-β-D-galacto-2-nonulopyranosonate (14). To a stirred solution of Neu5Ac (1) (10.0 g, 32.4 mmol) in MeOH (100 mL), 1.5 g of H resin was added, and the mixture was stirred at room temperature for 16 hours. The resin was filtered, and the reaction mixture was concentrated by evaporation in vacuo. Methylated Neu5Ac was dissolved in a stirred solution in pyridine (60 mL) at 0° C. Acetic anhydride (60 mL) was added, and the resulting mixture was stirred at room temperature for 18 hours. The solvent was then removed by evaporation in vacuo, and the residue was purified by silica gel column chromatography (hexane:EtOAc=1:9, by volume) to produce the protected methyl ester (14) (15.0 g, 87% yield) as a white solid. ¹H NMR (800 MHz, CDCl₃) δ 5.39-5.37 (m, 0.8H), 5.36-5.35 (m, 0.2H), 5.32-5.21 (m, 1.8H), 5.19 (ddd, J=7.2, 6.4, 3.2 Hz, 0.2H), 5.07 (ddd, J=6.4, 5.6, 2.4 Hz, 0.8H), 5.01 (ddd, J=12.0, 10.4, 4.8 Hz, 0.2H), 4.69 (dd, J=10.4, 2.4 Hz, 0.2H), 4.49 (dd, J=12.8, 2.4 Hz, 0.8H), 4.35 (dd, J=12.8, 3.2 Hz, 0.2H), 4.14-4.09 (m, 3H), 4.05 (dd, J=12.0, 5.6 Hz, 0.2H), 3.79 (s, 2.4H), 3.76 (s, 0.6H), 2.55 (dt, J=12.8, 4.8 Hz, 1H), 2.14 (d, J=3.2 Hz, 4H), 2.09 (d, J=3.2 Hz, 1H), 2.06 (s, 2.2H), 2.05-2.02 (m, 6.4H), 1.89 (s, 3H), 1.26 (t, J=7.2 Hz, 1H). ¹³C NMR (200 MHz, CDCl₃) δ 170.6, 170.4, 170.1, 161.5, 145.1, 107.5, 75.6, 70.6, 67.7, 61.9, 57.4, 52.6, 48.8, 23.4, 20.9, 20.8, 20.7. HRMS (ESI-Orbitrap) m/z calculated for C₂₃H₃₂NO₁₆⁻[M+FA−H]⁻578.1727, found 578.1714.

Methyl 7,8,9-tri-O-acetyl-2,3,4,5-tetradeoxy-2,3-didehydro-2,3-trideoxy-4′,5′-dihydro-2′-methyloxazolo[5,4-D]-D-glycero-D-talo-non-2-enonate (15). To the stirred solution of the protected methyl ester (14) (15.0 g, 28.1 mmol) in DCM (150 mL), BF₃·Et₂O (20.8 mL, 168.6 mmol) was added drop-wisely, and the mixture was stirred at room temperature under monitored with thin layer chromatography (TLC) for 14 hours. The reaction mixture was then diluted with ethyl acetate and washed gradually with saturated NaHCO₃, water, and brine. The solvent was concentrated in vacuo and the residue was purified with a flash silica gel column chromatography (hexane:EtOAc=1:4, by volume) to form the 4,5-oxazoline product 15 (10.5 g, 90% yield) as a white foam. ¹H NMR (800 MHz, CDCl₃) δ 6.37 (dd, J=4.0, 0.8 Hz, 1H), 5.67-5.60 (m, 1H), 5.49-5.40 (m, 1H), 4.81 (dd, J=8.8, 4.0, 1H), 4.58 (dd, J=12.0, 2.4 Hz, 1H), 4.21 (dd, J=12.8, 6.4 Hz, 1H), 3.94 (t, J=9.6 Hz, 1H), 3.80 (s, 3H), δ 3.42 (dd, J=10.4, 2.4 Hz, 1H), 2.14 (s, 3H), 2.05 (s, 3H), 2.04 (s, 3H), 1.99 (s, 3H). ¹³C NMR (200 MHz, CDCl₃) δ 170.6, 169.8, 169.6, 167.2, 161.8, 147.1, 107.5, 76.7, 72.2, 70.2, 68.8, 62.0, 61.9, 52.5, 20.8, 20.8, 20.6, 14.1.

Methyl 5-acetamido-7,8,9-tri-O-acetyl-4-azido-2,6-anhydro-3,4,5-trideoxy-D-glycero-D-galacto-non-2-enonate (16). The 4,5-oxazoline intermediate (15) (10.0 g, 24.2 mmol) was directly dissolved in dry tert-butanol (100 mL), TMSN₃ (6.4 mL, 48.4 mmol) was added, and the mixture was stirred at 80° C. for 14 hours. After the reaction finished and cooled down, aqueous NaNO₂ (1.0 g in 10 mL water) was added. HCl (1 M) was then added dropwise to the mixture until effervescence had ceased. The mixture was diluted with ethyl acetate and washed gradually with water, saturated NaHCO₃, and brine. The solvent was concentrated by evaporation in vacuo, and the residue was purified by silica gel column chromatography (hexane:EtOAc=2:4, by volume) to form the 4-azide derivative 16 (8.9 g, 81% yield) as a white solid. ¹H NMR (800 MHz, CDCl₃) δ 5.98 (d, J=2.4 Hz, 1H), 5.79 (d, J=8.0 Hz, 1H), 5.45 (dd, J=5.6, 2.4 Hz, 1H), 5.35-5.31 (m, 1H), 4.61 (dd, J=12.8, 3.2 Hz, 1H), 4.52 (dd, J=9.6, 2.4 Hz, 1H), 4.48 (dd, J=8.8, 2.4 Hz, 1H), 4.19 (dd, J=12.0, 6.4 Hz, 1H), 3.85-3.81 (m, 1H), 3.80 (s, 3H), 2.13 (s, 3H), 2.07 (s, 3H), 2.05 (s, 3H), 2.00 (s, 3H). ¹³C NMR (200 MHz, CDCl₃) δ 170.6, 170.4, 170.1, 161.5, 145.1, 107.5, 75.6, 70.6, 67.7, 61.9, 57.4, 52.6, 48.8, 23.4, 20.9, 20.8, 20.7. HRMS (ESI-Orbitrap) m/z calculated for C₁₉H₂₅N₄O₁₂⁻[M+FA−H]⁻501.1474, found 501.1463.

Synthesis of 4N₃Neu5Ac2en (5). The protected 4N₃Neu5 Ac2en methyl ester 16 (80 mg, 1 equiv.) was dissolved in MeOH and water (1:1) and treated with NaOH (pH >9), the reaction was monitored until completion by TLC (EtOAc:MeOH:H₂O=5:3:1) and confirmed with HRMS. The reaction mixture was concentrated, and the product 5 was purified by a Bio-gel P-2 column to form a light yellow solid (52.6 mg, 95% yield). ¹H NMR (800 MHz, D₂O) δ 5.70 (d, J=1.6 Hz, 1H), 4.32 (dd, J=9.6, 2.4 Hz, 1H), 4.29 (d, J=11.2 Hz, 1H), 4.23-4.19 (m, 1H), 3.94 (ddd, J=8.8, 6.4, 2.4 Hz, 1H), 3.89 (dd, J=12.0, 3.2 Hz, 1H), 3.66-3.62 (m, 2H), 2.07 (s, 3H). ¹³C NMR (200 MHz, D₂O) δ 174.5, 169.1, 149.3, 103.3, 75.2, 69.7, 68.0, 63.1, 59.2, 47.7, 22.1. HRMS (ESI-Orbitrap) m/z calculated for C₁₁H₁₅N₄O₇⁻[M−H]⁻ 315.0946, found 315.0940.

Synthesis of 4NH₂Neu5Ac2en (3). The 4N₃Neu5 Ac2en (5) (30 mg, 1 equiv.) obtained above was dissolved in water and treated with 1M of trimethylphosphine (PMe₃) in THF (0.2 mL, 2.0 equiv.). After 4 M NaOH solution was added to adjust the pH of the reaction to over 8.0. The reaction was stirred at room temperature for 3 hours and was monitored until completion by TLC (EtOAc:MeOH:H₂O=5:4:1) and confirmed with HRMS. The solvent was removed and purified using bio-gel P-2 column to form the product 3 as a white solid (23.6 mg, 86% yield). ¹H NMR (800 MHz, D₂O) δ 5.96 (d, J=2.4 Hz, 1H), 4.43 (d, J=10.4 Hz, 1H), 4.39 (t, J=9.6 Hz, 1H), 4.26 (dd, J=8.8, 2.4 Hz, 1H), 3.95 (ddd, J=8.8, 5.6, 2.4 Hz, 1H), 3.88 (dd, J=12.0, 3.2 Hz, 1H), 3.72 (d, J=9.6, 1H), 3.67 (dd, J=11.2, 5.6 Hz, 1H), 2.08 (s, 3H). ¹³C NMR (200 MHz, D₂O) δ 174.8, 165.3, 103.6, 103.5, 75.5, 69.7, 67.6, 62.9, 49.8, 45.6, 22.1. HRMS (ESI-Orbitrap) m/z calculated for C₁₁H₁₇N₂O₇⁻ [M−H]⁻ 289.1041, found 289.1034.

Synthesis of 4NAcNeu5Ac2en (6). The conversion from 4-azido group to the 4-acetamido group was achieved by dissolving the protected 4N₃Neu5Ac2en methyl ester 16 (40 mg, 1 equiv.) in pyridine (0.5 mL) with thioacetic acid (0.5 mL, 40 equiv.). Then the compound was de-protected using NaOH (reaction's pH >9) and a mixture solvent of MeOH and water (1:1). The reaction was monitored until completion by TLC (EtOAc:MeOH:H₂O=5:3:1) and confirmed with HRMS. The reaction mixture was concentrated upon completion and purified using a Bio-gel P-2 column to form compound 6 as a white syrup (27.3 mg, 94% yield). ¹H NMR (600 MHz, D₂O) δ 5.56 (d, J=1.8 Hz, 1H), 4.77 (d, J=2.4 Hz, 1H), 4.34 (dd, J=10.8, 1.2 Hz, 1H), 4.12 (t, J=10.2 Hz, 1H), 3.97 (ddd, J=9.0, 6.6, 2.4 Hz, 1H), 3.91 (dd, J=12.0, 3.0 Hz, 1H), 3.68-3.64 (m, 2H), 2.01 (s, 3H), 2.00 (s, 3H), 1.92 (s, 2H). ¹³C NMR (150 MHz, D₂O) δ 174.4, 174.2, 169.5, 148.8, 105.5, 75.4, 69.8, 68.2, 63.1, 48.2, 47.9, 22.0, 21.9. HRMS (ESI-Orbitrap) m/z calculated for C₁₃H₁₉N₂O₈⁻ [M−H]⁻ 331.1147, found 331.1137.

Methyl 5-acetamido-2,7,8,9-tetra-O-acetyl-4-amino-3,4,5-trideoxy-D-glycero-β-D-galacto-2-nonulopyranosonate (17). To the stirred solution of 4-azide derivative 16 (7.32 g, 16.1 mmol) in AcOH (36 mL) containing NaOAc (3.60 g), N-bromosuccinimide (3.40 g, 19.2 mmol) was added and the mixture was stirred at room temperature for 20 min. The reaction mixture was then diluted with DCM and washed gradually with brine, saturated NaHCO₃, and brine. The solvent was concentrated in vacuo to give the crude product as a white solid, which contains two 3-Br isomers. The crude product was then directly dissolved in 1,6-dioxane (100 mL), Bu₃SnH (18.1 mL, 67.2 mmol) was added and AIBN (279.2 mg, 1.7 mmol) in dioxane was added drop-wisely, and the mixture was heated at 80° C. for 3 hours. The solvent was concentrated in vacuo and extracted with acetonitrile and hexane. The residue obtained was purified by silica gel column chromatography (EtOAc:MeOH=2:1, by volume) to form the protected 4-amine derivative 17 (3.3 g, 42%) as a white solid. ¹H NMR (800 MHz, CDCl₃) δ 5.96 (d, J=9.6 Hz, 1H), 5.34 (dd, J=4.8, 2.4 Hz, 1H), 5.00 (ddd, J=7.2, 4.8, 2.4 Hz, 1H), 4.47 (dd, J=12.8, 2.4 Hz, 1H), 4.08 (dd, J=12.4, 6.9 Hz, 1H), 4.02 (dd, J=10.4, 2.4 Hz, 1H), 3.73 (s, 3H), 3.63 (dd, J=10.4, 20.8 Hz, 1H), 3.15-3.06 (m, 1H), 2.40 (dd, J=13.6, 4.0 Hz, 1H), 2.07 (s, 3H), 2.07 (s, 3H), 2.00 (s, 3H), 1.98 (s, 3H), 1.93 (s, 3H), 1.72 (dd, J=13.6, 12.0 Hz, 1H), 0.85 (t, J=7.2 Hz, 1H). ¹³C NMR (200 MHz, CDCl₃) δ 171.2, 170.6, 170.4, 170.4, 168.4, 167.0, 97.6, 72.7, 71.7, 68.3, 62.2, 53.0, 52.6, 48.6, 39.2, 23.3, 20.9, 20.8, 20.8, 20.7. HRMS (ESI-Orbitrap) m/z calculated for C₂₁H₃₁N₂O₁₄⁻ [M+FA−H]⁻535.1781, found 535.1769.

4NH₂Neu5Ac (7). To the stirred solution of protected 4-amine derivative 17 (3.0 g, 6.1 mmol) in MeOH (10 mL) and H₂O (10 mL), aqueous NaOH (4 M) was added drop-wisely until the pH reached to 10. The reaction was neutralized after 30 minutes by aqueous HCl (1 M). The crude product was purified by a bio-gel P-2 column to form 1.3 g of compound 7 with 67% yield. ¹H NMR (800 MHz, D₂O) δ 4.06 (d, J=10.4 Hz, 1H), 3.98 (t, J=10.4 Hz, 1H), 3.84 (dd, J=12.0, 3.2 Hz, 1H), 3.76 (ddd, J=9.6, 6.4, 3.2 Hz, 1H), 3.64-3.59 (m, 1H), 3.52 (d, J=9.6 Hz, 1H), 3.43-3.39 (m, 1H), 2.19 (dd, J=12.8, 4.0 Hz, 1H), 2.06 (s, 3H), 1.87 (t, J=12.8 Hz, 1H). ¹³C NMR (200 MHz, D₂O) δ 176.48, 174.90, 95.50, 70.16, 70.01, 68.43, 63.21, 50.32, 48.50, 37.37, 22.08. HRMS (ESI-Orbitrap) m/z calculated for [M−H]⁻ 307.1147, found 307.1139.

Synthesis of Neu5Acα2-3Gal/LacNAcβProNH-PEG4-Biotin (8c-d) and Neu5Acα2-6Gal/LacNAcβProNH-PEG4-Biotin (8e-f). The acceptor (GalβProNH-PEG4-Biotin 18 or LacNAcβProNH-PEG4-Biotin 19) (20-30 mg), Neu5Ac (1.5 equiv.), and CTP (1.5 equiv.) were dissolved in water in a 50 mL centrifuge tube containing Tris-HCl buffer (100 mM, pH 8.5) and MgCl₂ (20 mM). After adding NmCSS (0.5 mg), and PmST1_M144D (1 mg, for producing α2-3-linked sialosides) or Psp2,6ST_A366G (1 mg, for producing α2-6linked sialosides), water was added to bring the final concentration of acceptor to 10 mM. The reaction mixture was incubated at 30° C. for 16 h. The reaction progress was monitored using mass spectrometry. After 16 to 20 h, the reaction mixture was incubated in a boiling water bath for 5 min to denature the enzymes, cooled down to room temperature, then centrifuged at 8000 rpm for 30 min at 4° C. The supernatant was concentrated and purified by a preconditioned DSC-18 SPE cartridge (bed wt. 10 g) eluting with MeOH in water (from 0 to 100%) to form the desired product.

Neu5Acα2-3GalβProNH-PEG4-Biotin (8c). 39 mg, 91% yield. ¹H NMR (800 MHz, D₂O) δ 4.60 (dd, J=8.0, 4.8 Hz, 1H), 4.45 (d, J=8.0 Hz, 1H), 4.42 (dd, J=8.0, 4.0 Hz, 1H), 4.08 (dd, J=9.6, 3.2 Hz, 1H), 3.98-3.52 (m, 30H), 3.38 (t, J=5.4 Hz, 2H), 3.35-3.26 (m, 3H), 3.19 (d, J=7.0 Hz, 1H), 2.99 (dd, J=12.8, 4.8 Hz, 1H), 2.79-2.73 (m, 2H), 2.51 (t, J=6.4 Hz, 2H), 2.27 (t, J=7.2 Hz, 2H), 2.02 (s, 3H), 1.86-1.77 (m, 3H), 1.75-1.56 (m, 5H), 1.44-1.38 (m, 2H), 1.27 (d, J=6.4 Hz, 1H). ¹³C NMR (200 MHz, D₂O) δ 176.91, 174.98, 173.83, 165.31, 102.51, 99.81, 75.82, 74.88, 72.82, 71.74, 69.62, 69.60, 69.58, 69.54, 69.48, 69.39, 69.15, 68.85, 68.33, 68.03, 67.64, 67.48, 66.78, 62.53, 62.05, 60.95, 60.22, 55.32, 51.65, 39.66, 39.62, 38.87, 36.42, 36.04, 35.43, 28.36, 27.84, 27.66, 25.10, 22.00, 21.98. HRMS (ESI-Orbitrap) m/z: [M−H]⁻ calculated for C₄₁H₇₀N₅O₂₁S 1000.4289; found 1000.4308.

Neu5Acα2-3LacNAcβProNH-PEG4-Biotin (8d). 31 mg, 92% yield. ¹H NMR (800 MHz, D₂O) δ 4.62-4.57 (m, 2H), 4.54 (d, J=8.0 Hz, 1H), 4.49 (d, J=8.0 Hz, 1H), 4.45-4.40 (m, 2H), 4.11-3.53 (m, 44H), 3.38 (t, J=5.6 Hz, 2H), 3.36-3.14 (m, 7H), 3.01-2.95 (m, 2H), 2.76-2.46 (m, 5H), 2.27 (t, J=7.4 Hz, 2H), 2.03 (s, 3H), 2.02 (s, 3H), 1.83-1.52 (m, 6H), 1.41-1.38 (m, 3H). 13C NMR (200 MHz, D₂O) δ 183.77, 179.37, 176.89, 174.97, 174.44, 173.85, 165.37, 165.30, 102.54, 101.08, 99.78, 78.31, 75.44, 75.14, 74.75, 72.85, 72.37, 71.73, 69.62, 69.61, 69.60, 69.58, 69.55, 69.48, 69.39, 69.36, 69.27, 68.85, 68.32, 68.05, 67.92, 67.67, 67.44, 66.78, 62.54, 62.05, 61.95, 61.00, 60.22, 60.21, 60.17, 60.03, 55.33, 55.31, 55.29, 55.05, 51.65, 46.63, 39.67, 39.66, 39.59, 38.87, 37.59, 37.28, 36.21, 36.02, 35.43, 28.32, 28.22, 27.85, 27.82, 27.67, 27.65, 27.63, 25.62, 25.11, 25.09, 24.85, 22.13, 22.00. HRMS (ESI-Orbitrap) m/z: [M−H]⁻ calculated for C₄₉H₈₃N₆O₂₆S 1203.5083; found 1203.5105.

Neu5Acα2-6GalβProNH-PEG4-Biotin (8e). 38 mg, 89% yield. ¹H NMR (800 MHz, D₂O) δ 4.60 (dd, J=8.0, 4.0 Hz, 1H), 4.42 (dd, J=8.0, 4.8 Hz, 1H), 4.36 (d, J=8.0 Hz, 1H), 3.98-3.73 (m, 10H), 3.71-3.54 (m, 16H), 3.49 (dd, J=10.4, 8.0 Hz, 1H), 3.41-3.23 (m, 6H), 2.98 (dd, J=12.8, 4.8 Hz, 1H), 2.77 (d, J=13.6 Hz, 1H), 2.72 (dd, J=12.8, 4.8 Hz, 1H), 2.52 (t, J=5.6 Hz, 2H), 2.27 (t, J=7.2 Hz, 2H), 2.02 (s, 3H), 1.85-1.80 (m, 2H), 1.76-1.55 (m, 6H), 1.44-1.38 (m, 2H). ¹³C NMR (200 MHz, D₂O) δ 176.91, 175.03, 173.87, 173.37, 165.31, 102.81, 100.41, 73.41, 72.61, 72.54, 71.71, 70.66, 69.62, 69.60, 69.58, 69.55, 69.48, 69.39, 68.85, 68.56, 68.16, 67.73, 66.78, 63.35, 62.59, 62.05, 60.22, 55.31, 51.83, 40.20, 39.66, 38.87, 36.27, 36.06, 35.42, 29.55, 28.38, 27.84, 27.65, 25.10, 21.98. HRMS (ESI-Orbitrap) m/z: [M−H]⁻ calculated for C₄₁H₇₀N₅O₂₁S 1000.4289; found 1000.4310.

Neu5Acα2-6LacNAcβProNH-PEG4-Biotin (8f). 30 mg, 90% yield. ¹H NMR (800 MHz, D₂O) δ 4.61-4.58 (m, 1H), 4.53 (d, J=8.0 Hz, 1H), 4.45-4.40 (m, 2H), 4.00-3.50 (m, 41H), 3.42-3.15 (m, 7H), 3.02-2.95 (m, 1H), 2.79-2.62 (m, 3H), 2.51 (t, J=6.4 Hz, 2H), 2.27 (t, J=7.2 Hz, 1H), 2.05 (s, 3H), 2.02 (s, 3H), 1.80-1.54 (m, 8H), 1.41 (q, J=7.2 Hz, 2H). ¹³C NMR (200 MHz, D₂O) δ 183.75, 176.89, 174.88, 174.46, 173.84, 173.50, 165.30, 103.47, 100.88, 100.11, 80.77, 74.46, 73.66, 72.53, 72.45, 72.39, 71.67, 70.70, 69.62, 69.60, 69.58, 69.57, 69.55, 69.53, 69.48, 69.39, 68.85, 68.37, 68.33, 68.18, 67.67, 66.78, 63.30, 62.62, 62.05, 61.95, 60.36, 60.22, 60.21, 59.53, 55.33, 55.29, 54.83, 51.86, 46.64, 40.06, 39.67, 38.88, 37.27, 36.24, 36.02, 35.43, 28.35, 28.21, 27.85, 27.67, 27.63, 25.62, 25.11, 22.24, 22.00. HRMS (ESI-Orbitrap) m/z: [M−H]⁻ calculated for C₄₉H₈₃N₆O₂₆S 1203.5083; found 1203.5107.

One-pot two-enzyme (OP2E) synthesis of 4NH₂Neu5Ac-containing sialosides (9a-d). An acceptor (1 equiv., 10 mM), sialic acid 4NH₂Neu5Ac (5) (21-87 mg, 1.5 equiv.), and CTP (56-254 mg, 2.5 equiv.) were dissolved in water in a 50 mL centrifuge tube containing Tris-HCl buffer (100 mM, pH 8.5), MgCl₂ (20 mM), NmCSS (0.5 mg), and a sialyltransferase PmST1 (2 mg) or Psp2,6ST_A366G (3 mg). The reaction mixture was incubated at 30° C. for 4 h for α2-3-sialylation or 36 h for α2-6-sialylation. The reaction progress was monitored using TLC (EtOAc/MeOH/H₂O=3:5:1, by volume) and ultra-high-performance liquid chromatography (UHPLC). After the reaction reached completion, the reaction was cooked in a boiling water bath for 5 minutes. The resulting mixture was centrifuged, and the supernatant was concentrated by evaporation in vacuo. The final compounds (9a-9f) were purified using a reverse phase C₁₈ column as white foams.

4NH₂Neu5Acα2-3GalβpNP (9a). 108.0 mg, 92% yield. ¹H NMR (800 MHz, D₂O) δ 8.27-8.25 (m, 2H), 7.37-7.12 (m, 2H), 5.30 (d, J=8.0 Hz, 1H), 4.29 (dd, J=9.6, 3.2 Hz, 1H), 4.17 (t, J=10.4 Hz, 1H), 4.06 (d, J=3.2 Hz, 1H), 3.96-3.89 (m, 3H), 3.87-3.81 (m, 2H), 3.77 (d, J=6.4 Hz, 2H), 3.66-3.62 (m, 2H), 3.39 (dd, J=12.0, 4.8 Hz, 1H), 2.86 (dd, J=12.8, 4.4 Hz, 1H), 2.04 (s, 3H), 2.02 (t, J=12.8 Hz, 1H). ¹³C NMR (200 MHz, D₂O) δ 175.0, 173.0, 161.7, 142.5, 126.1, 126.1, 116.4, 116.4, 99.7, 99.3, 75.4, 75.4, 73.1, 71.6, 68.8, 67.8, 67.1, 62.5, 60.6, 50.5, 47.8, 35.6, 22.1. HRMS (ESI-Orbitrap) m/z calculated for C₂₃H₃₂N₃O₁₅⁻ [M−H]⁻ 590.1839, found 590.1837.

4NH₂Neu5Acα2-6GalβpNP (9b). 83.0 mg, 90% yield. ¹H NMR (800 MHz, D₂O) δ 8.35-8.22 (m, 2H), 7.35-7.17 (m, 2H), 5.20 (d, J=8.0 Hz, 1H), 4.04 (d, J=4.0 Hz, 1H), 4.03 (d, J=4.8 Hz, 1H), 3.98-3.96 (m, 1H), 3.88-3.83 (m, 3H), 3.82-3.76 (m, 3H), 3.70 (dd, J=10.4, 4.0 Hz, 1H), 3.61 (dd, J=12.0, 6.4 Hz, 1H), 3.56 (dd, J=8.8, 1.6 Hz, 1H), 3.06-2.97 (m, 1H), 2.72 (dd, J=12.8, 4.0 Hz, 1H), 2.03 (s, 3H), 1.65 (t, J=12.8 Hz, 1H). ¹³C NMR (200 MHz, D₂O) δ 175.1, 173.3, 161.8, 142.5, 126.1, 126.1, 116.4, 116.4, 100.1, 99.8, 74.0, 73.2, 72.3, 71.7, 70.2, 68.4, 68.1, 63.1, 62.6, 50.4, 49.8, 38.4, 22.0. HRMS (ESI-Orbitrap) m/z calculated for C₂₃H₃₂N₃O₁₅⁻ [M−H]⁻590.1839, found 590.1840.

4NH₂Neu5Acα2-3GalβPorNH-PEG4-Biotin (9c). 40.1 mg, 95% yield. ¹H NMR (600 MHz, D₂O) δ 4.62 (dd, J=8.4, 5.4 Hz, 1H), 4.47 (d, J=7.8 Hz, 1H), 4.44 (dd, J=7.8, 4.2 Hz, 1H), 4.16-4.08 (m, 2H), 3.99-3.94 (m, 2H), 3.94-3.86 (m, 2H), 3.83-3.78 (m, 3H), 3.77-3.62 (m, 20H), 3.57 (dd, J=9.6, 7.8 Hz, 1H), 3.41 (t, J=5.4 Hz, 2H), 3.38-3.27 (m, 4H), 3.01 (dd, J=12.6, 4.8 Hz, 1H), 2.84-2.77 (m, 2H), 2.54 (t, J=6.0 Hz, 2H), 2.29 (t, J=7.2 Hz, 2H), 2.06 (s, 3H), 1.97 (t, J=12.6 Hz, 1H), 1.85 (p, J=6.6 Hz, 2H), 1.78-1.72 (m, 1H), 1.70-1.57 (m, 3H), 1.46-1.41 (m, 2H). ¹³C NMR (150 MHz, D₂O) δ 176.9, 175.0, 173.9, 173.2, 165.3, 102.5, 99.3, 75.8, 74.9, 73.1, 71.6, 69.6, 69.6, 69.6, 69.6, 69.5, 69.4, 69.2, 68.9, 67.8, 67.6, 67.3, 66.8, 62.5, 62.1, 60.9, 60.2, 55.4, 50.4, 48.2, 39.7, 38.9, 36.4, 36.1, 35.9, 35.5, 28.4, 27.9, 27.7, 25.1, 22.1. HRMS (ESI-Orbitrap) m/z calculated for C₄₁H₇₁N₆O₂₀S⁻ [M−H]⁻ 999.4449, found 999.4447.

4NH₂Neu5Acα2-3LacNAcβPorNH-PEG4-Biotin (9d). 30.3 mg, 92% yield. ¹H NMR (800 MHz, D₂O) δ 4.61 (dd, J=8.0, 4.8 Hz, 1H), 4.55 (d, J=8.0 Hz, 1H), 4.50 (d, J=8.0 Hz, 1H), 4.43 (dd, J=8.0, 4.0 Hz, 1H), 4.11 (dd, J=9.6, 3.2 Hz, 1H), 3.99 (dd, J=12.0, 2.4 Hz, 1H), 3.95 (d, J=3.2 Hz, 1H), 3.94-3.88 (m, 2H), 3.88-3.83 (m, 2H), 3.79-3.72 (m, 6H), 3.71-3.66 (m, 16H), 3.66-3.63 (m, 1H), 3.63-3.61 (m, 3H), 3.60-3.55 (m, 3H), 3.39 (dd, J=6.4, 4.8 Hz, 2H), 3.35-3.32 (m, 1H), 3.27 (dt, J=13.6, 7.2 Hz, 1H), 3.18 (dt, J=13.6, 7.2 Hz, 1H), 2.99 (dd, J=12.8, 4.8 Hz, 1H), 2.90-2.81 (m, 1H), 2.78 (d, J=12.8 Hz, 1H), 2.66 (dd, J=12.8, 4.0 Hz, 1H), 2.52 (t, J=6.4 Hz, 2H), 2.28 (t, J=7.2 Hz, 2H), 2.06-2.01 (m, 6H), 1.80-1.75 (m, 2H), 1.75-1.55 (m, 5H), 1.46-1.38 (m, 2H). ¹³C NMR (200 MHz, D₂O) δ 176.9, 175.1, 174.4, 173.9, 165.3, 102.6, 101.1, 99.8, 78.3, 75.4, 75.2, 74.8, 73.8, 72.4, 71.7, 69.6, 69.6, 69.6, 69.5, 69.5, 69.4, 69.4, 69.4, 68.9, 68.1, 67.7, 67.4, 66.8, 62.5, 62.1, 61.0, 60.2, 60.0, 55.3, 55.0, 49.6, 39.7, 38.9, 36.2, 36.0, 35.4, 28.3, 27.8, 27.7, 25.1, 22.1, 22.0. HRMS (ESI-Orbitrap) m/z calculated for C₄₉H₈₄N₇O₂₅S⁻ [M−H]⁻ 1202.5243, found 1202.5223.

General methods for converting 4NH₂Neu5Ac-containing sialosides (9a-d) to 4-guanidino-Neu5Ac-containing sialosides (10a-d). The 4NH₂Neu5Ac-containing sialoside (9a-d) (12 mg, 1 equiv.) was dissolved in 1 mL of anhydrous THF and 2 mL of MeOH. 1,3-Di-Boc-2-(trifluoromethylsulfonyl) guanidine (15-23 mg, 3 equiv.) was added to the mixture and triethylamine was added to adjust the pH to around 9. The reaction was stirred at 37° C. for 24 h and was monitored by TLC (iPrOH:H₂O:NH₄OH=7:2:1, by volume). After the reaction was completed, the solvent was removed and 1.5 mL of trifluoroacetic acid was added. The reaction was stirred vigorously for 30-60 min at room temperature. After the reaction was completed, the solvent was removed by air dry and purified by a reverse phase C18 column. The sialosides were eluted at 20-60% acetonitrile in water, followed by lyophilization to obtain pure compounds (10a-d) as white foams.

4-Guanidino-Neu5Acα2-3GalβpNP (10a). 11.0 mg, 86% yield. ¹H NMR (600 MHz, D₂O) δ 8.35-8.23 (m, 2H), 7.46-7.16 (m, 2H), 5.32 (d, J=7.8 Hz, 1H), 4.30 (dd, J=9.6, 3.0 Hz, 1H), 4.08-4.03 (m, 2H), 3.97-3.90 (m, 3H), 3.89-3.82 (m, 2H), 3.78 (d, J=6.0 Hz, 2H), 3.70-3.61 (m, 3H), 2.76 (dd, J=12.6, 4.2 Hz, 1H), 2.01 (s, 3H), 1.98 (dd, J=13.2, 12.0 Hz, 1H). ¹³C NMR (150 MHz, D₂O) δ 174.6, 173.6, 161.7, 156.6, 142.6, 127.8, 126.1, 116.4, 115.9, 99.7, 99.4, 75.4, 75.4, 73.0, 71.7, 68.8, 68.0, 67.1, 62.5, 60.6, 51.7, 49.9, 37.0, 21.8. HRMS (ESI-Orbitrap) m/z calculated for C₂₄H₃₄N₅O₁₅⁻ [M−H]⁻ 632.2057, found 632.2059.

4-Guanidino-Neu5Acα2-6GalβpNP (10b). 10.3 mg, 81% yield. ¹H NMR (600 MHz, D₂O) δ 8.312-8.29 (m, 2H), 7.30-7.27 (m, 2H), 5.23 (d, J=7.8 Hz, 1H), 4.06-4.03 (m, 2H), 4.02-3.99 (m, 1H), 3.92 (t, J=9.6 Hz, 1H), 3.87 (m, 4H), 3.81 (dd, J=10.2, 3.2 Hz, 1H), 3.72 (dd, J=10.2, 3.6 Hz, 1H), 3.65-3.62 (m, 1H), 3.62-3.60 (m, 1H), 3.60-3.55 (m, 1H), 2.68 (dd, J=13.2, 4.2 Hz, 1H), 1.99 (s, 3H), 1.78 (dd, J=13.2, 12.0 Hz, 1H). ¹³C NMR (150 MHz, D₂O) δ 174.6, 173.3, 161.8, 156.5, 142.5, 126.1, 125.5, 116.4, 115.4, 100.0, 99.6, 74.1, 72.7, 72.3, 71.6, 70.3, 68.5, 68.1, 63.3, 62.5, 51.6, 50.0, 37.3, 21.8. HRMS (ESI-Orbitrap) m/z calculated for C₂₄H₃₄N₅O₁₅⁻ [M−H]⁻ 632.2057, found 632.2050.

4-Guanidino-Neu5Acα2-3Galβ PorNH-PEG4-Biotin (10c). 10.2 mg, 82% yield. ¹H NMR (800 MHz, D₂O) δ 4.61 (dd, J=8.0, 4.8 Hz, 1H), 4.46 (d, J=8.0 Hz, 1H), 4.42 (dd, J=8.0, 4.8 Hz, 1H), 4.12 (dd, J=9.6, 3.2 Hz, 1H), 4.02 (t, J=9.6 Hz, 1H), 3.98-3.93 (m, 2H), 3.91-3.89 (m, 1H), 3.87 (dd, J=12.0, 2.4 Hz, 1H), 3.81 (d, J=10.4 Hz, 1H), 3.78 (t, J=6.4 Hz, 2H), 3.76-3.70 (m, 3H), 3.69 (s, 8H), 3.68-3.67 (m, 4H), 3.66-3.61 (m, 6H), 3.57-3.52 (m, 1H), 3.39 (t, J=5.6 Hz, 2H), 3.35-3.32 (m, 1H), 3.32-3.27 (m, 2H), 2.99 (dd, J=12.8, 4.8 Hz, 1H), 2.78 (d, J=12.8.0 Hz, 1H), 2.70 (dd, J=13.6, 4.8 Hz, 1H), 2.52 (t, J=5.6 Hz, 2H), 2.27 (t, J=7.2 Hz, 2H), 1.99 (s, 3H), 1.94 (t, J=12.0 Hz, 1H), 1.84 (q, J=7.2 Hz, 2H), 1.71-1.75 (m, 1H), 1.68-1.56 (m, 3H), 1.45-1.38 (m, 2H). ¹³C NMR (200 MHz, D₂O) δ 176.9, 174.6, 173.9, 173.8, 165.3, 156.6, 102.5, 75.7, 74.9, 72.9, 71.6, 69.6, 69.6, 69.6, 69.5, 69.5, 69.4, 69.2, 68.9, 68.0, 67.6, 67.3, 66.8, 62.5, 62.1, 60.9, 60.2, 55.3, 51.6, 49.9, 39.7, 38.9, 36.7, 36.4, 36.0, 35.4, 28.4, 27.8, 27.7, 25.1, 23.2, 21.8. HRMS (ESI-Orbitrap) m/z calculated for C₄₂H₇₃N₈O₂₀S⁻ [M−H]⁻ 1041.4667, found 1041.4652.

4-Guanidino-Neu5Acα2-3LacNAcβPorNH-PEG4-Biotin (10d). 7.3 mg, 88% yield. ¹H NMR (800 MHz, D₂O) δ 4.61 (dd, J=8.0, 4.8 Hz, 1H), 4.55 (d, J=8.0 Hz, 1H), 4.51 (d, J=8.0 Hz, 1H), 4.42 (dd, J=8.0, 4.8 Hz, 1H), 4.14 (dd, J=10.4, 3.2 Hz, 1H), 4.02 (t, J=9.6 Hz, 1H), 3.99 (dd, J=12.8, 2.4 Hz, 1H), 3.96 (d, J=3.2 Hz, 1H), 3.93-3.89 (m, 2H), 3.87 (dd, J=12.0, 2.4 Hz, 1H), 3.85-3.80 (m, 2H), 3.78 (t, J=5.6 Hz, 2H), 3.75-3.71 (m, 4H), 3.71-3.70 (m, 3H), 3.69 (s, 8H), 3.67 (s, 3H), 3.65-3.61 (m, 6H), 3.59-3.55 (m, 2H), 3.41-3.37 (m, 2H), 3.36-3.32 (m, 1H), 3.27 (dt, J=13.6, 6.4 Hz, 1H), 3.18 (dt, J=13.6, 7.2 Hz, 1H), 2.99 (dd, J=13.6, 5.6 Hz, 1H), 2.78 (d, J=12.8 Hz, 1H), 2.69 (dd, J=13.6, 4.8 Hz, 1H), 2.51 (t, J=5.6 Hz, 2H), 2.27 (t, J=7.2 Hz, 2H), 2.03 (s, 3H), 1.99 (s, 3H), 1.97-1.90 (m, 1H), 1.79-1.75 (m, 2H), 1.75-1.70 (m, 1H), 1.69-1.56 (m, 3H), 1.46-1.37 (m, 2H). ¹³C NMR (200 MHz, D₂O) δ 176.9, 174.6, 174.5, 173.9, 173.7, 165.3, 161.0, 156.6, 102.5, 101.1, 99.4, 78.3, 75.3, 75.1, 74.7, 72.9, 72.4, 71.6, 69.6, 69.6, 69.6, 69.5, 69.5, 69.4, 68.9, 68.0, 67.7, 67.2, 66.8, 62.5, 62.1, 61.0, 60.2, 55.3, 55.1, 51.6, 49.9, 39.7, 38.9, 36.2, 36.0, 35.4, 28.3, 27.8, 27.7, 25.1, 22.1, 21.8. HRMS (ESI-Orbitrap) m/z calculated for C₅₀H₈₆N₉O₂₅S⁻ [M−H]⁻ 1244.5461, found 1244.5435.

General methods for converting 4NH₂Neu5Ac-containing sialosides (9a-d) to 4N₃Neu5Ac-containing sialosides (11a-d). A 4NH₂Neu5 Ac-containing sialoside (9a-d) (12 mg, 1 equiv.) was dissolved in 0.5 mL H₂O and 1 mL MeOH containing 15 mg of K₂CO₃ and 10 mg of CuSO₄·5H₂O, freshly prepared TfN₃ from NaN₃ (25 equiv.) was then added to the mixture and the reaction was stirring at room temperature for 16 h. The reaction was monitored by TLC (EtOAc:MeOH:H₂O=5:3:1, by volume). After the reaction reached completion, the solvent was removed by rotavapor, and the final products were purified by a reverse phase C18 column. The sialosides were eluted at 20-60% acetonitrile in water, followed by evaporation in vacuo and lyophilization to produce pure compounds (11a-d) as white foams.

4N₃Neu5Acα2-3GalβpNP (11a). 10.4 mg, 83% yield. ¹H NMR (600 MHz, D₂O) δ 8.37-8.23 (m, 2H), 7.32-7.16 (m, 2H), 5.32 (d, J=7.8 Hz, 1H), 4.27 (dd, J=10.2, 3.0 Hz, 1H), 4.07 (d, J=3.0 Hz, 1H), 4.02 (t, J=10.2 Hz, 1H), 3.96-3.88 (m, 3H), 3.86 (dd, J=12.0, 2.4 Hz, 1H), 3.81-3.76 (m, 3H), 3.68-3.59 (m, 3H), 2.82 (dd, J=12.6, 4.2 Hz, 1H), 2.06 (s, 3H), 1.85 (t, J=12.6 Hz, 1H); ¹³C NMR (150 MHz, D₂O) δ 174.8, 173.4, 161.7, 142.5, 127.0, 125.4, 116.4, 114.7, 99.7, 99.6, 75.5, 75.4, 73.2, 71.8, 68.8, 68.0, 67.2, 62.5, 60.7, 59.6, 49.8, 36.8, 22.0. HRMS (ESI-Orbitrap) m/z calculated for C₂₃H₃₀N₅O₁₅⁻ [M−H]⁻ 616.1744, found 616.1737.

4N₃Neu5Acα2-6GalβpNP (11b). 10.9 mg, 87% yield. ¹H NMR (600 MHz, D₂O) δ 8.38-8.23 (m, 2H), 7.31-7.24 (m, 2H), 5.21 (dd, J=7.8, 1.2 Hz, 1H), 4.06-4.04 (m, 1H), 4.05-4.02 (m, 2H), 3.92-3.84 (m, 4H), 3.83-3.80 (m, 2H), 3.72-3.69 (m, 1H), 3.64-3.57 (m, 3H), 2.77 (dd, J=12.6, 4.2 Hz, 1H), 2.04 (s, 3H), 1.67 (t, J=12.6 Hz, 1H). ¹³C NMR (150 MHz, D₂O) δ 174.8, 173.2, 161.8, 142.5, 126.1, 125.6, 116.4, 113.3, 100.0, 99.8, 74.0, 72.9, 72.3, 71.7, 70.3, 68.4, 68.0, 63.0, 62.6, 59.4, 49.9, 37.2, 22.0. HRMS (ESI-Orbitrap) m/z calculated for C₂₃H₃₀N₅O₁₅⁻ [M−H]⁻ 616.1744, found 616.1756.

4N₃Neu5Acα2-3GalβProNH-PEG4-Biotin (11c). 6.0 mg, 84% yield. ¹H NMR (800 MHz, D₂O) δ 4.61 (dd, J=8.0, 4.8 Hz, 1H), 4.45 (d, J=8.0 Hz, 1H), 4.43 (dd, J=8.0, 4.8 Hz, 1H), 4.09 (dd, J=10.4, 3.2 Hz, 1H), 4.01-3.93 (m, 3H), 3.90-3.84 (m, 2H), 3.78 (t, J=6.4 Hz, 2H), 3.76-3.66 (m, 18H), 3.62 (q, J=5.6, 4.0 Hz, 4H), 3.55 (t, J=8.8 Hz, 1H), 3.39 (t, J=5.6 Hz, 2H), 3.35-3.33 (m, 1H), 3.33-3.27 (m, 2H), 2.99 (dd, J=12.8, 4.8 Hz, 1H), 2.78 (d, J=12.8 Hz, 1H), 2.77-2.75 (m, 1H), 2.52 (t, J=5.6 Hz, 2H), 2.28 (t, J=7.2 Hz, 2H), 2.04 (s, 3H), 1.86-1.82 (m, 2H), 1.82-1.79 (m, 1H), 1.75-1.71 (m, 1H), 1.69-1.56 (m, 3H), 1.45-1.38 (m, 2H). ¹³C NMR (200 MHz, D₂O) δ 176.9, 174.8, 173.9, 173.4, 165.3, 102.5, 101.2, 75.8, 74.9, 73.2, 71.7, 69.6, 69.6, 69.6, 69.5, 69.5, 69.4, 69.1, 68.9, 67.9, 67.7, 67.5, 66.8, 62.5, 62.1, 60.9, 60.2, 59.6, 55.3, 49.8, 39.7, 38.9, 36.7, 36.4, 36.0, 35.4, 28.4, 27.8, 27.7, 25.1, 22.0. HRMS (ESI-Orbitrap) m/z calculated for C₄₁H₆₉N₈O₂₀S⁻ [M−H]⁻ 1025.4354, found 1025.4345.

4N₃Neu5Acα2-3LacNAcβProNH-PEG4-Biotin (11d). 6.2 mg, 87% yield. ¹H NMR (800 MHz, D₂O) δ 4.61 (dd, J=8.0, 4.8 Hz, 1H), 4.54 (d, J=7.2 Hz, 1H), 4.50 (d, J=8.8 Hz, 1H), 4.43 (dd, J=8.0, 4.8 Hz, 1H), 4.12 (dd, J=10.4, 3.2 Hz, 1H), 4.01-3.96 (m, 2H), 3.96 (d, J=3.2 Hz, 1H), 3.94-3.91 (m, 1H), 3.91-3.88 (m, 1H), 3.88-3.85 (dd, J=11.9, 2.5 Hz, 1H), 3.84 (dd, J=12.0, 4.8 Hz, 1H), 3.78 (t, J=5.6 Hz, 2H), 3.75-3.69 (m, 7H), 3.69-3.66 (m, 12H), 3.66-3.61 (m, 5H), 3.61-3.55 (m, 3H), 3.39 (t, J=4.8 Hz, 2H), 3.36-3.31 (m, 1H), 3.3-3.24 (m, 1H), 3.21-3.15 (m, 1H), 2.99 (dd, J=12.8, 4.8 Hz, 1H), 2.78 (d, J=12.8 Hz, 1H), 2.76 (dd, J=12.8, 4.8 Hz, 1H), 2.52 (t, J=5.6 Hz, 2H), 2.28 (t, J=7.2 Hz, 2H), 2.09-1.09 (m, 6H), 1.83-1.69 (m, 4H), 1.70-1.55 (m, 3H), 1.46-1.38 (m, 2H). ¹³C NMR (200 MHz, D₂O) δ 176.9, 174.8, 174.4, 173.9, 173.5, 165.3, 102.5, 101.1, 99.6, 78.3, 75.4, 75.1, 74.7, 73.2, 72.4, 71.7, 69.6, 69.6, 69.6, 69.5, 69.5, 69.4, 69.3, 68.9, 67.9, 67.7, 67.4, 66.8, 62.5, 62.1, 61.0, 60.2, 60.0, 59.6, 55.3, 55.0, 49.8, 39.7, 38.9, 36.7, 36.2, 36.0, 29.6, 28.3, 27.8, 27.7, 25.1, 22.1, 22.0. HRMS (ESI-Orbitrap) m/z calculated for C₄₉H₈₂N₉O₂₅S⁻ [M−H]⁻ 1228.5148, found 1228.5128.

General Methods for converting 4NH₂Neu5Ac-containing sialosides (9a-d) to 4NAcNeu5Ac-containing sialosides (12a-d). The 4NH₂Neu5Ac-containing sialoside (9a-d) (30 mg, 1 equiv.) was dissolved in 1 mL of methanol, acetic anhydride (10-50 μL, 10 equiv.) was added, 100 μL of triethylamine was then used to adjust the pH to around 9.0. The reaction was stirred at room temperature and was monitored by TLC (EtOAc:MeOH:H₂O=5:4:1, by volume). After the reaction was completed, the reaction mixture was dried, re-dissolved in 1 mL of H₂O, the pH was adjusted to 11.0 and the reaction container was put on vacuum to remove the triethylamine, followed by purification via a reverse phase C18 column. The final products were eluted with 20-60% acetonitrile in water. After evaporation in vacuo and lyophilization, the pure 4-N-acetyl sialosides (12a-d) were obtained as white foams.

4NAcNeu5Acα2-3GalβpNP (12a). 31.5 mg, 98% yield. ¹H NMR (800 MHz, D₂O) δ 8.28-8.26 (m, 2H), 7.27-7.25 (m, 2H), 5.31 (d, J=8.0 Hz, 1H), 4.27 (dd, J=9.6, 3.2 Hz, 1H), 4.06 (d, J=3.2 Hz, 1H), 4.0-3.96 (m, 1H), 3.95-3.88 (m, 4H), 3.85 (dd, J=12.0, 2.4 Hz, 1H), 3.82 (dd, J=9.6, 1.6 Hz, 1H), 3.77 (s, 1H), 3.77 (s, 1H), 3.63-3.61 (m, 2H), 2.66 (dd, J=12.8, 4.0 Hz, 1H), 1.97-1.96 (m, 6H), 1.86 (t, J=12.8 Hz, 1H). ¹³C NMR (200 MHz, D₂O) δ 174.6, 173.7, 173.6, 161.7, 142.5, 126.1, 126.1, 116.4, 116.4, 99.7, 99.7, 75.4, 75.4, 73.6, 71.8, 68.8, 68.1, 67.1, 62.5, 60.7, 49.7, 48.5, 37.1, 21.9, 21.9. HRMS (ESI-Orbitrap) m/z calculated for C₂₅H₃₄N₃O₁₆⁻ [M−H]⁻ 632.1945, found 632.1947.

4NAcNeu5Acα2-6GalβpNP (12b). 30.5 mg, 95% yield. ¹H NMR (800 MHz, D₂O) δ 8.32-8.28 (m, 2H), 7.29-7.25 (m, 2H), 5.21 (d, J=8.0 Hz, 1H), 4.03-4.02 (m, 2H), 4.0-3.97 (m, 1H), 3.95-3.92 (m, 1H), 3.88-3.80 (m, 6H), 3.68 (dd, J=10.4, 3.2 Hz, 1H), 3.61 (dd, J=12.8, 7.2 Hz, 1H), 3.57 (dd, J=8.8, 1.6 Hz, 1H), 2.61 (dd, J=12.8, 4.0 Hz, 1H), 1.95 (s, 3H), 1.94 (s, 3H), 1.66 (t, J=12.8 Hz, 1H). ¹³C NMR (200 MHz, D₂O) δ 174.6, 173.7, 173.3, 161.8, 142.5, 126.1, 126.1, 116.4, 116.4, 100.2, 99.7, 74.0, 73.2, 72.3, 71.7, 70.3, 68.4, 68.2, 63.1, 62.6, 49.8, 48.3, 37.6, 21.9, 21.8. HRMS (ESI-Orbitrap) m/z calculated for C₂₅H₃₄N₃O₁₆⁻ [M−H]⁻ 632.1945, found 632.1948.

4NAcNeu5Acα2-3GalβProNH-PEG4-Biotin (12c). 5.2 mg, 99% yield. ¹H NMR (800 MHz, D₂O) δ 4.61 (dd, J=8.0, 5.6 Hz, 1H), 4.46 (d, J=8.0 Hz, 1H), 4.42 (dd, J=8.0, 4.8 Hz, 1H), 4.10 (dd, J=9.6, 3.2 Hz, 1H), 3.97-3.94 (m, 3H), 3.913-3.90 (m, 1H), 3.90-3.84 (m, 2H), 3.79-3.77 (m, 3H), 3.75-3.70 (m, 3H), 3.69-3.66 (m, 13H), 3.65-3.61 (m, 4H), 3.54 (dd, J=9.6, 8.0 Hz, 1H), 3.39 (t, J=5.6 Hz, 2H), 3.36-3.32 (m, 1H), 3.32-3.27 (m, 2H), 2.99 (dd, J=13.6, 5.6 Hz, 1H), 2.78 (d, J=12.8 Hz, 1H), 2.61 (dd, J=12.8, 4.0 Hz, 1H), 2.52 (t, J=6.4 Hz, 2H), 2.28 (t, J=7.2 Hz, 2H), 1.96 (s, 6H), 1.86-1.80 (m, 3H), 1.76-1.70 (m, 1H), 1.69-1.56 (m, 3H), 1.45-1.38 (m, 2H). ¹³C NMR (200 MHz, D₂O) δ 176.9, 174.5, 173.9, 173.7, 173.6, 165.3, 102.5, 99.7, 75.8, 74.9, 73.5, 71.8, 69.6, 69.6, 69.6, 69.5, 69.5, 69.4, 69.2, 68.9, 68.0, 67.6, 67.4, 66.8, 62.5, 62.1, 61.0, 60.2, 55.3, 49.7, 48.5, 39.7, 38.9, 37.1, 36.4, 36.0, 35.4, 28.4, 27.8, 27.7, 25.1, 21.9, 21.9. HRMS (ESI-Orbitrap) m/z calculated for C₄₃H₇₃N₆O₂₁S⁻ [M−H]⁻ 1041.4555, found 1041.4552.

4NAcNeu5Acα2-3LacNAcβProNH-PEG4-Biotin (12d). 5.2 mg, 98% yield. ¹H NMR (800 MHz, D₂O) δ 4.61 (dd, J=8.0, 5.6 Hz, 1H), 4.55 (d, J=7.2 Hz, 1H), 4.50 (d, J=8.8 Hz, 1H), 4.42 (dd, J=8.0, 4.8 Hz, 1H), 4.12 (dd, J=9.6, 3.2 Hz, 1H), 3.99 (d, J=12.0 Hz, 1H), 3.98-3.88 (m, 5H), 3.87 (dd, J=12.0, 2.4 Hz, 1H), 3.84 (dd, J=12.0, 4.8 Hz, 1H), 3.80-3.76 (m, 3H), 3.75-3.70 (m, 6H), 3.69 (s, 8H), 3.67 (s, 3H), 3.64-3.59 (m, 5H), 3.59-3.55 (m, 2H), 3.39 (t, J=5.6 Hz, 2H), 3.33 (dt, J=9.6, 4.8 Hz, 1H), 3.27 (dt, J=13.6, 7.2 Hz, 1H), 3.18 (dt, J=13.6, 6.4 Hz, 1H), 2.99 (dd, J=12.8, 4.8 Hz, 1H), 2.78 (d, J=12.8 Hz, 1H), 2.61 (dd, J=12.8, 4.0 Hz, 1H), 2.51 (t, J=6.4 Hz, 2H), 2.27 (t, J=7.2 Hz, 2H), 2.03 (s, 3H), 1.96 (s, 6H), 1.92-1.89 (m, 1H), 1.82 (t, J=12.0 Hz, 1H), 1.78-1.75 (m, 2H), 1.75-1.70 (m, 1H), 1.69-1.56 (m, 3H), 1.47-1.38 (m, 2H). ¹³C NMR (200 MHz, D₂O) δ 176.9, 174.5, 174.5, 173.9, 173.7, 165.3, 161.6, 102.6, 101.1, 99.7, 78.3, 75.4, 75.1, 74.7, 73.6, 72.4, 71.7, 69.6, 69.6, 69.6, 69.5, 69.5, 69.4, 69.4, 68.9, 68.1, 67.7, 67.3, 66.8, 62.5, 62.1, 61.0, 60.2, 60.1, 55.3, 55.1, 48.5, 39.7, 38.9, 37.0, 36.2, 36.0, 35.4, 28.3, 27.8, 27.7, 25.1, 23.2, 22.1, 21.9, 21.9. HRMS (ESI-Orbitrap) m/z calculated for C₅₁H₈₆N₇O₂₆S⁻ [M−H]⁻ 1244.5349, found 1244.5329.

High-throughput substrate specificity assays. These assays were carried out in duplicate in a 384-well plate with a final volume of 20 μL. Each sialoside (0.3 mM) was incubated with a sialidase and an excess amount of β-galactosidase at 37° C. for 30 min. High-concentration sialidase assays were carried out using 4-fold (Vc Sialidase) or 10-fold (other sialidases) sialidases for 1 h and 14 h. Assays were stopped with 40 μL of 0.5 M CAPS buffer (pH 11.5), and A_{405 nm} readings were obtained by a microplate reader. For every sialoside tested, duplicate reactions without a sialidase were used as negative controls and for background readings. The sialidase amounts and buffers used were: hNEU2 (0.6 μg), MES buffer (100 mM, pH 5.0); BiNanH2 (0.1 μg), NaOAc buffer (100 mM, pH 5.0); A. ureafaciens sialidase (1.0 mU), NaOAc buffer (100 mM, pH 5.5); C. perfringens sialidase (0.6 mU), MES buffer (100 mM, pH 5.0); V. cholerae sialidase (2.8 mU), NaCl (150 mM), CaCl₂) (10 mM), NaOAc buffer (100 mM, pH 5.5); SpNanA (14 ng), NaOAc buffer (100 mM, pH 6.0); SpNanB (5 ng), NaOAc buffer (100 mM, pH 6.0); SpNanC (20 ng), MES buffer (100 mM, pH 6.5); PmST1 (0.5 μg), NaOAc buffer (100 mM, pH 5.5), CMP (0.4 mM);. Amounts 4-fold (Vc Sialidase) or 10-fold (other sialidases) higher were used for high-concentration sialidase assays. Buffer condition for IAV and IBV NAs: NaCl (150 mM), CaCl₂) (1 mM), MES buffer (25 mM, pH 6.0). Amounts for low concentration sialidase assays: N1-Vic19 (0.08 μg), N1-BR18 (0.06 μg), N2-Dar21 (0.06 μg), N2-Kan17 (0.10 μg), NB—NB—Aus21 (0.02 μg), NB-Phu13 (0.015 μg). Amounts of 10-fold higher were used for high-concentration sialidase assays.

Inhibition assays. These assays were performed in duplicates in a 96-well plate with a final volume of 80 μL. Each reaction contains Neu5Acα2-3GalβMU (8.0 μM), an excess amount of β-galactosidase (12 μg), with or without an inhibitor (8.0 μM). The assay conditions were: hNEU2 (2.8 μg), MES buffer (100 mM, pH 5.0); N1-BR18 (0.06 μg), NaCl (150 mM), CaCl₂) (1 mM), MES buffer (25 mM, pH 6.0). The reactions were carried out for 30 min and quenched with 80 μL of N-cyclohexyl-3-aminopropane sulfonic acid buffer (0.5 M, pH 11.5). The fluorescence of free MU formed was determined using a microplate reader with excitation at 360 nm and emission at 460 nm. Inhibition assays for obtaining IC₅₀ values were carried out in duplicates in a 96-well plate similarly as described above except that a series of different concentrations of inhibitors were used. IC₅₀ values were obtained by fitting the obtained fluorescence values to the inhibitor concentration-response plots using GraphPad Prism 9.

Enzyme-linked immunosorbent assays (ELISA). These assays were carried out in duplicates in 384-well NeutrAvidin-coated plates. The wells were washed with 3×80 μL of washing buffer (1× PBS buffer with 0.05% tween-20) and then were incubated with biotinylated glycans (10 μM, 20 μL) at 4° C. for overnight. Then the wells were washed with 3×80 μL of washing buffer and blocked with 60 μL of blocking buffer (1× PBS buffer with 0.1% BSA). The wells were then washed with 3×80 μL of washing buffer, and NA (10 μg/mL, 20 μL) was added to each well. The samples were incubated at 4° C. for 30 minutes. The wells were then washed with 3×80 μL of washing buffer, and HRP-conjugated anti-His or anti-Strep antibodies (0.1 μg/mL or with proper dilution using 1× PBS buffer with 0.1% BSA, 20 μL) were added. The samples were incubated at 4° C. for 30 minutes. After washing with 3×80 μL of the washing buffer, 20 μL of TMB substrate was added to each well and the plate was incubated at room temperature for 30 minutes. The reaction was then stopped with 20 μL of 2 M sulfuric acid and absorbance readings at 450 nm were measured by a plate reader.

Bio-lay interferometry (BLI) assays. The BLI assays were carried out at 26° C. by following a reported procedure¹² with modifications. The solutions containing biotinylated sialosides (100 nM, 200 μL each) were loaded to the wells of a 96-well plate. The analyte solutions of NAs (100 μL per well containing 1×Octet® kinetics buffer with or without 1 μM NA or its 3×serial diluted solutions) were loaded to the wells in a 384-well titled-bottom plate to minimize the materials required. Streptavidin (SAX) biosensor tips were hydrated in the kinetics buffer in a reservoir for 10 min before being mounted onto a sensor rack which was then placed in the Octet 384RED (Fortébio) sensor tray. The SAX biosensor tips were dipped into the biotinylated sialoside solutions (100 nM, 200 μL each in the 96-well plate) for 150 or 200 s or until the streptavidin biosensors reached coating saturation. After a baseline step of incubating the biosensor tips with the kinetics buffer (100 μL per well in the 384-well titled-bottom plate) for 60 s, the functionalized biosensor tips were dipped in the analyte solutions (100 μL per well in a titled bottom 384-well plate) containing various concentrations of NA (1 μM and 3×serial diluted solutions) for 400 s. The sensor tips were then switched to be dipped in the kinetics buffer (100 μL per well in the titled bottom 384-well plate) for 600 or 900 s to allow dissociation. The sensorgrams obtained were fitted to a 1:1 binding model using Octet Analysis software. The equilibrium responses at the end of the association phase (400 s) were used for steady state analysis by fitting to a “one site-specific binding” model on GraphPad Prism 9 to obtain the K_{D} values.

H1N1 virus detection by ELISA. HINI/BR18 virus was grown in 10-day old specific pathogen free embryonated eggs for 3 days at 33° C. and the allantoic fluid was harvested and clarified by sedimentation (4,000×g for 5 min). ELISA assays were carried out in duplicate using 96-well 2HB plates pre-coated with 25 μg/well streptavidin and blocked at 37° C. for 1 h with 200 μL of 1% BSA in PBS pH 7.4. Wells were washed with 3×200 μL of PBS pH 7.4 containing 0.1% Tween 20 (PBST) and incubated with 100 μL of the biotinylated glycans diluted to the indicated concentrations in PBST containing 0.1% BSA at 37° C. for 1 h. Wells were washed with 3×200 μL PBST and 100 μL of HINI/BR18 virus in allantoic fluid diluted 1:4 in PBST was added to the wells at 37° C. for 1 h. Wells were washed 6×200 μL with PBST and incubated with 100 μL of a rabbit polyclonal antibody against H1 (1 μg/mL) at 37° C. for 1 h. Wells were washed with 3×200 μL PBST and incubated with a HRP-conjugated goat-anti-rabbit secondary at a 1:20000 dilution at 37° C. for 1 h. Wells were washed again with 3×200 μL PBST and developed using 100 μL of OPD substrate at 37° C. for 10 min and stopped by adding 50 μL/well of 2 N sulfuric acid and absorbance readings at 490 nm were measured with a plate reader.

H1N1 virus isolation with streptavidin beads. Dynabeads MyOne streptavidin T1 (10 mg) were washed with 2×10 mL virus buffer (25 mM MES pH 7, 150 mM NaCl and 1 mM CaCl₂)), resuspended with 1 mL of virus buffer containing 20 μM of the biotinylated glycan and incubated at 37° C. for 1 h. Beads were washed with 2×10 mL virus buffer and resuspended in 1 mL virus buffer. Clarified H1N1/BR18 virus in allantoic fluid (~9 mL) was added to the beads and incubated at 37° C. for 1 h. Beads were sedimented (4,000×g for 5 min) and the supernatant containing the unbound virus was retained. Beads were washed with 2×10 mL virus buffer and bound virus was eluted by incubating the beads with 200 μL virus buffer containing 50 mM Neu5 Ac2en at 4° C. for overnight. A magnet was then used to pulldown the beads and the supernatant was removed.

HA titer determination, SDS-PAGE and DLS analysis. HA titers were determined using 0.5% turkey red blood cells (TRBCs). Briefly, 50 μL samples were diluted two-fold in 96-well round bottom plates using PBS. Following the dilution, 50 μL of 0.5% TRBCs were added to each well, the plate was incubated at room temperature for 30 min, and HA titers corresponded to the last well where agglutination was observed were recorded. Equal samples volumes were mixed with sample buffer with or without 0.1 M dithiotreitol (DTT), heated at 50° C. for 10 min and resolved using 4-12% SDS-PAGE gels. Gels were stained with SimpleBlue and imaged using an Azure C600 imaging system. Dynamic light scattering measurements were performed on the isolated virus diluted 1:10 in 0.2 μm filtered virus buffer (25 mM MES pH 7, 150 mM NaCl and 1 mM CaCl₂)) using a DynaPro NanoStar II. Data from three independent samples were collected using 10×5 sec intervals and each reading set was analyzed with the regularization setting to obtain the particle diameter and polydispersity measurements.

### Example 4. 8-N-Sialoside Substrate Specificity for Influenza Virus Neuraminidases

The numbering of compounds, schemes, and tables presented in this Example 4 is specific only to this Example 4. The chemoenzymatically synthesized 8N-sialosides (20-25) described above were used as probes with Neu5 Ac-sialosides including Neu5Acα2-3GalβpNP and Neu5Acα2-6GalβpNP as positive controls to investigate the substrate specificity of recombinant NAs from four (H1N1 and H3N2) IAV strains and two IBV strains, a recombinant human cytoplasmic sialidase hNEU2, and eight bacterial sialidases (FIG. 11). FIG. 11 shows sialidase substrate specificity studies using sialyl GalβpNPs with low (a) and high (b) enzyme concentrations.

The high-throughput colorimetric assays were carried out in a 384-well plate under two enzyme-concentration conditions. The enzyme concentrations in the low-enzyme-concentration conditions (FIG. 11a) were determined so that the reactions of producing para-nitrophenolate from Neu5Acα2-3GalβpNP as the control substrate reached only 30-80% completion indicated by an average A405 nm reading in the range of 0.3-0.8. The high-enzyme-concentration conditions (FIG. 11b) used ten-fold higher enzyme concentrations (FIG. 11b). Due to the limitation of the stock solution concentration of the commercially available Vibrio cholerae (Vc) sialidase, only its low-enzyme-concentration assays were carried out. In the assays, each sialidase was incubated with a sialoside in the presence of an excess amount of B-galactosidase at 37° C. for 30 min. The reaction was then stopped by adding two reaction volumes of cold N-cyclohexyl-3-aminopropanesulfonic acid (CAPS) buffer (pH 11.5, 0.5 M) to adjust the pH of the solution to above 9.5 to convert most of the para-nitrophenol formed to para-nitrophenolate and the A_{405 nm} of the resulting sample was recorded. In this assay, para-nitrophenol is only released by β-galactosidase when GalβpNP was produced by sialidase cleavage of the sialoside probe.

Results with both low (FIG. 11a) and high (FIG. 11b) sialidase concentration assay conditions showed that sialosides containing a C₈-azido modification on Neu5Ac were tolerated by all sialidases tested although significantly reduced catalytic efficiency was observed for a recombinant Bifidobacterium infantis sialidase (BiNanH2) and a commercially available Arthrobacter ureafaciens (Au) sialidase. 8N3Neu5Ac-sialosides were better substrates than their unmodified natural counterparts for hNEU2 and Streptococcus pneumoniae sialidase SpNanB.

Sialosides containing Neu5Ac with C8-NHAc or C8—NH₂ modification were not tolerated by most sialidases tested under the low-enzyme-concentration assay conditions (FIG. 11a). However, under the high-enzyme-concentration assay conditions (FIG. 11b) both were cleaved by several bacterial sialidases including commercially available Clostridium perfringens (Cp) sialidase, the α2-3-sialidase activity of the multifunctional sialyltransferase PmST1, and recombinant Streptococcus pneumoniae sialidases SpNanA, SpNanB, and SpNanC. Compared to the sialosides containing a C8-NHAc-modified Neu5Ac, those with a C8—NH₂-modified Neu5Ac had a broader tolerance and could be additionally cleaved by the NAs from two recent HINI IAV strains A/Victoria/2570/2019 (N1-Vic19) and A/Brisbane/02/2018 (N1-BR18) under the high-enzyme-concentration assay conditions (FIG. 11b). Under both assay conditions, we observed minimal or no activity when we tested the 8NAcNeu5Ac- and 8NH₂Neu5Ac-sialosides with hNEU2, BiNanH2, and Au Sialidase, as well as the NAs from recent H3N2 IAV strains A/Darwin/9/2021 (N2-Dar21) and A/Kansas/14/2017 (N2-Kan17), and the IBV strains B/Austria/1359417/2021 (NB-NB-Aus21) and B/Phuket/3073/2013 (NB-Phu13) (FIG. 11b).

Based on the substrate specificity results we performed a kinetic analysis on two of the recombinant IAV NAs (N1-Vic19 and N1-BR18) and two of the recombinant bacterial sialidases (SpNanA and SpNanB) that showed activities on a relatively broad range of sialosides containing different 8-N-modified sialic acids. The catalytic efficiencies for these four sialidases (Table S2, ESI) aligned well with the substrate specificity study results (FIG. 11) and showed that SpNanA and SpNanB were more efficient catalysts compared to the two HINI IAV NAs.

A comparison of the sialidase substrate profiles from sialosides containing C8-N₃/NHAc modified Neu5 Ac and the corresponding C7-modified counterparts showed that sialosides containing Neu5 Ac with either C8-N₃ and C7-N₃ modifications were tolerated by both bacterial sialidases and the NAs from influenza viruses while hNEU2 could only tolerate the C8-N₃ modification. Sialosides with Neu5Ac containing a C8-NHAc modification were not cleaved by the NAs from influenza viruses, whereas those with C7-NHAc-modified Neu5Ac were previously shown to be susceptible to cleavage by influenza NAs under high-enzyme-concentration assay conditions. In comparison, sialosides containing C9-N₃/NHAc modified Neu5Ac were suitable substrates for the NAs from IAVs and numerous bacterial sialidases. Together, these results suggest that a selected collection of sialosides containing different sialic acids can be used to profile sialidases and viral neuraminidases for diagnostic purposes.

### Example 5. Synthesis of 4-N-Sialosides

The numbering of compounds, schemes, and tables presented in this Example 5 is specific only to this Example 5. To obtain target sialosides containing a diverse array of 4-N-derivatized Neu5Ac, 4NH₂Neu5 Ac (8) was designed as a precursor compound. Scheme 1 shows the synthesis of 4NH₂Neu5Ac (8). Compound 8 was prepared from commercially available Neu5Ac (1) in eight steps via a fully protected 4-azido Neu5 Ac2en intermediate (Scheme 1). Briefly, the carboxyl group in Neu5Ac (1) was methylated and its hydroxyl groups were protected by acetylation to form per-O-acetylated methyl ester of Neu5 Ac (11) in 90% yield in two steps. Treatment of 11 with BF₃·Et₂O in dichloromethane (DCM) at room temperature formed oxazoline 12 (95% yield), which was reacted with azido trimethylsilane (TMSN₃) to produce 4-azido methyl ester 13. Treatment of 13 with N-bromosuccinimide (NBS), followed by debromination and reduction using tri-n-butyltinhydride, yielded 4-amino methyl ester 14 in 42% yield in two steps. Deprotection of 14 with sodium methoxide in methanol and water formed the desired 4NH₂Neu5Ac (8). The identity and the purity of 4NH₂Neu5Ac (8) was confirmed by high-resolution mass spectrometry (HRMS) and nuclear magnetic resonance (NMR) analyses.

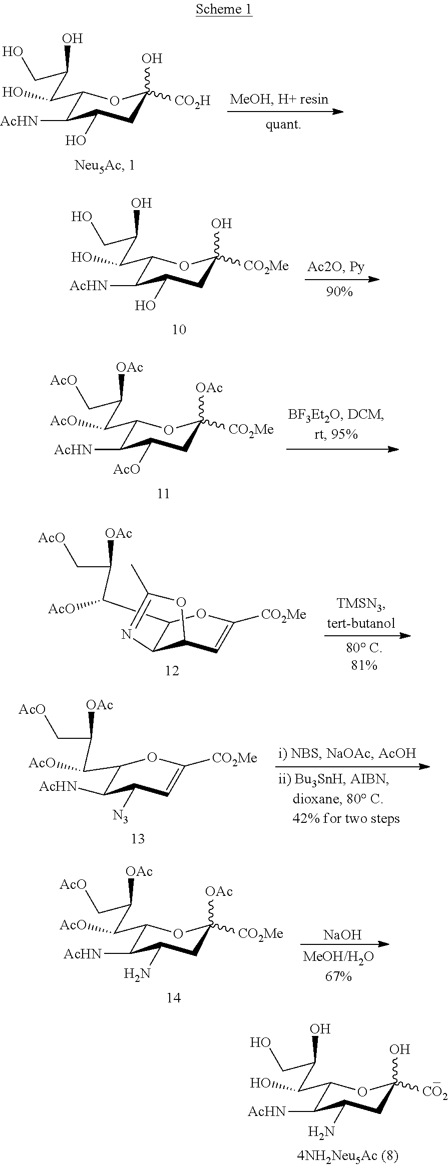

After synthesizing 4NH₂Neu5Ac (8), para-nitrophenol (pNP)-tagged sialosides 4NH₂Neu5Acα2-3GalβpNP (15a) and 4NH₂Neu5Acα2-6GalβpNP (15b) (Scheme 2) were prepared in 92% and 82% yields, respectively, using a one-pot two-enzyme sialylation system containing Neisseria meningitidis cytidine 5′-monophosphate (CMP)-sialic acid synthetase (NmCSS) and Pasteurella multocida α2-3-sialyltransferase-1 (PmST1) or Photobacterium sp. α2-6-sialyltransferase A366G mutant (Psp2,6ST_A366G).

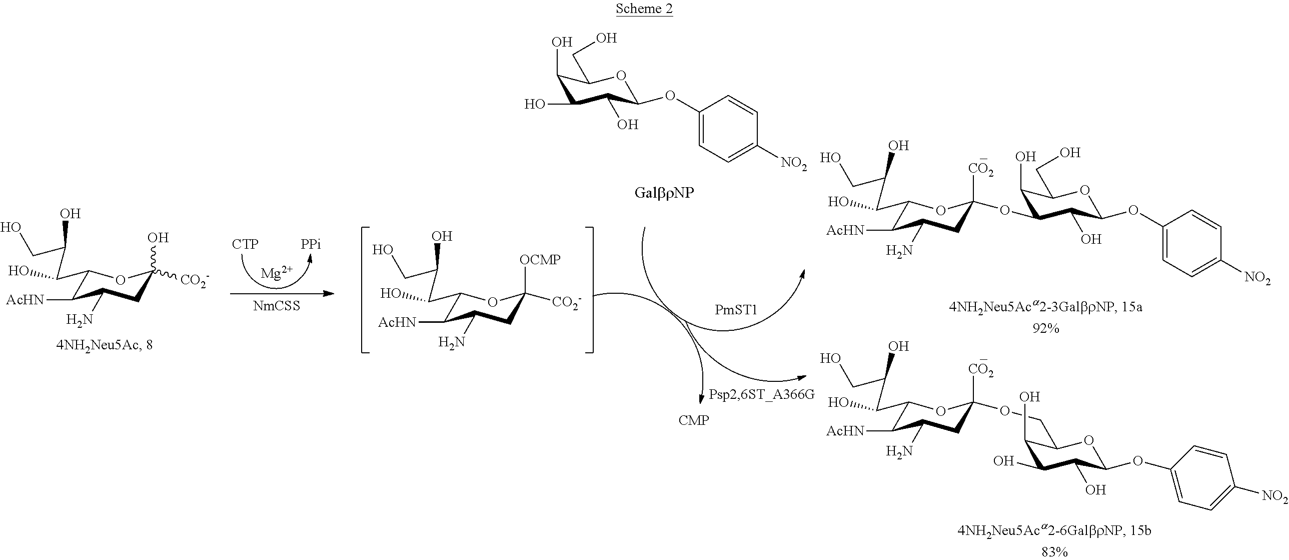

Derivatizing the C₄—NH₂ group in these pNP-tagged sialosides (15a/b) formed sialosides containing other C4-modified Neu5 Ac derivatives (16a/b-18a/b) (Scheme 3). 4NAcNeu5Acα2-3/6GalβpNP (16a/b) were synthesized in 98% and 95% yields, respectively, by treating 4NH₂Neu5Acα2-3/6GalβpNP (15a/b) with acetic anhydride under a basic condition in triethylamine. 4N3Neu5Acα2-3/6GalβpNP (17a/b) were obtained in 83% and 87% yields, respectively, by treating 4NH₂Neu5Acα2-3/6GalβpNP (15a/b) with freshly prepared TfN₃ in a mixed solvent of H₂O and methanol. 4-Guanidino-Neu5Acα2-3/6GalβpNP (18a/b) were obtained in 86% and 81% yields, respectively, by treating 4NH₂Neu5Acα2-3/6GalβpNP (15a/b) with 1,3-di-Boc-2-(trifluoromethylsulfonyl) guanidine in a mixed solution of anhydrous dichloromethane and methanol under a basic reaction condition in triethylamine, followed by removing the Boc protecting group in 50% of trifluoroacetic acid in anhydrous dichloromethane.

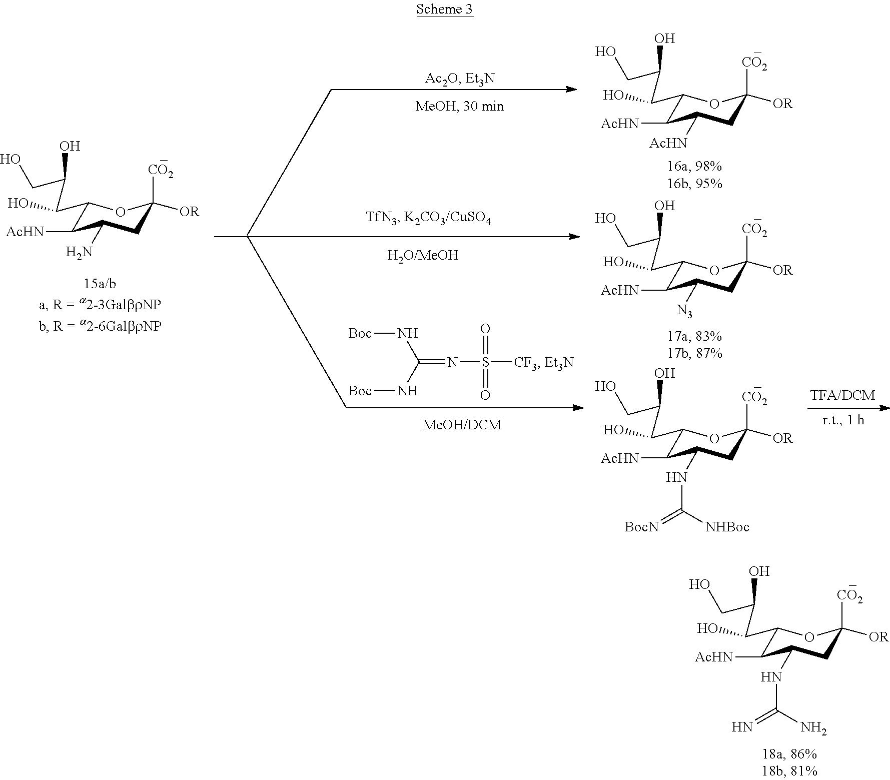

### Example 6. Synthesis of 8-N-Sialosides

The numbering of compounds, schemes, and tables presented in this Example 6 is specific only to this Example 6.

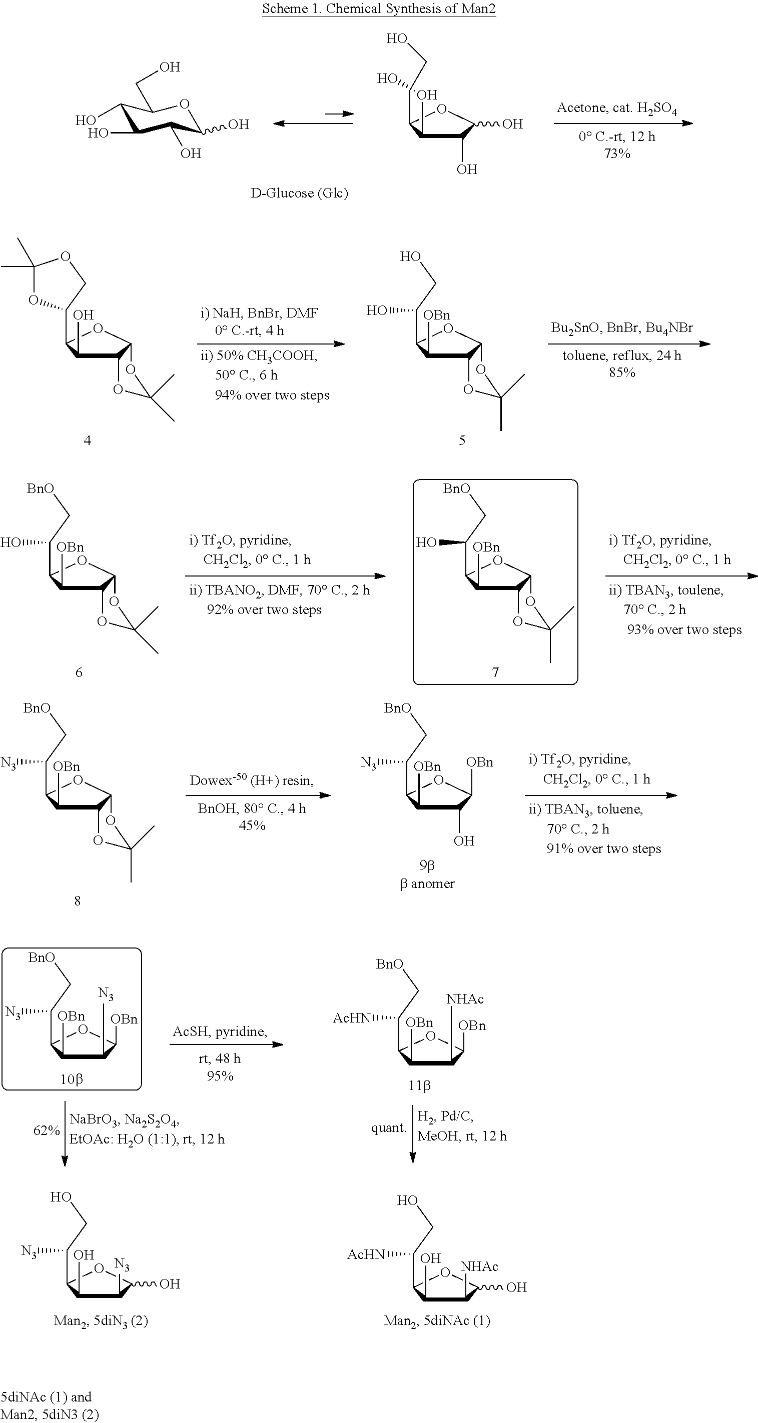

Scheme 1 shows the chemical synthesis of the enzymatic precursor Man2,5diNAc (1) and a chemoenzymatic synthon Man2,5diN3 (2) from D-glucose (Glc) for OP3E synthesis of 8NAcNeu5Ac-sialosides.

To synthesize Man2,5diNAc (1, Scheme 1), D-glucose was locked in a furanose form using an acid-catalyzed acetalization to form 1,2:5,6-di-O-isopropylidene-a-D-glucofuranose (4) in 73% yield. Benzylation of the C3-OH in the glucofuranoside 4 followed by selective deprotection of the 5,6-isopropylidene by mild acid hydrolysis with 50% acetic acid formed a diol (5) with an overall yield of 94% over two steps. Regio-selective benzylation of the C6—OH of the diol 5 via the formation of a dibutylstannylene derivative followed by benzylation with benzyl bromide resulted in compound 6 with a single unprotected OH at C5 in 85% yield. Treatment of 6 with triflic anhydride (Tf₂O) and pyridine followed by displacing the triflate group at C5 with a hydroxyl group using tetrabutylammonium nitrite (TBANO₂) in dry dimethylformamide (DMF) at 70° C. formed the L-idose derivative 7 in 92% yield with the inversion of the C5 stereochemistry. Another round of inversion at the C5 of the compound 7 by reacting with Tf₂O and pyridine followed by displacing the triflate group at C5 with an azido group using tetrabutylammonium azide (TBAN₃) in dry toluene at 70° C. produced the azido-D-glucose derivative 8 in 93% yield.

Treatment of compound 8 with benzyl alcohol as a solvent in the presence of an acidic cation exchange resin produced benzyl furanoside as an α/β mixture. The β-anomer 9β (45% yield) was purified from the reaction mixture using silica gel column chromatography. The a-isomer was also purified from benzyl alcohol. The C2-OH of the β-anomer 9β was converted to a triflate ester by treating with Tf₂O and pyridine followed by azido inversion using TBAN₃ in toluene to produce per-O-benzylated 2,5-diazido-2,5-dideoxy-mannofuranoside (10) in 91% yield with the inversion of the stereochemistry at C2. The benzyl protected diazido-mannose compound 10β was subject to the oxidative cleavage of the benzyl group in the presence of sodium bromate and sodium dithionite in an ethyl acetate-water biphasic solvent system. Under these conditions, the chemoenzymatic synthon Man2,5diN₃ (2) was obtained in a reasonable yield (62%) that was lower than the reaction yield (85-90%) observed by thin-layer chromatography (TLC) analysis, possibly due to the instability of the product during the purification process. On the other hand, treatment of 10β with thioacetic acid in pyridine produced per-O-benzylated Man2,5diNAc (11B) in 95% yield which was readily converted to the desired Man2,5diNAc (1) in quantitative yield by catalytic hydrogenation with H₂ and Pd/C.

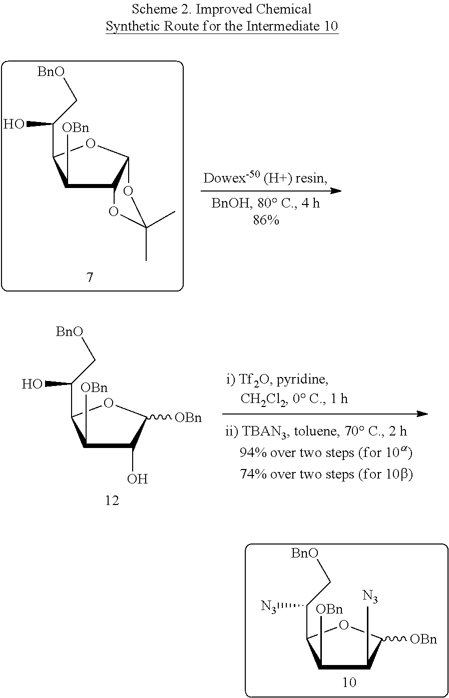

Scheme 2 shows the improved synthesis of compound 10. The α- and β-anomers of the diazido intermediate 10 both could be used for the synthesis of Man2,5diNAc (1) and Man2,5diN₃ (2) and the efficiency for producing this key intermediate from compound 7 was improved by shortening the synthetic route (Scheme 2). This was achieved by opening the 1,2-isopropylidene ring of compound 7 using benzyl alcohol in the presence of an acidic cation exchange resin to produce a diol 12 as an α- and β-mixture in 86% yield. The anomers were purified readily using silica gel column chromatography and converted to the corresponding diazido compounds 10α and 10β with 94% and 74% yields (over two steps), respectively, with simultaneous inversion of the stereocenters at C2 and C5 by treating with Tf₂O and pyridine, followed by azido inversion using TBAN₃.

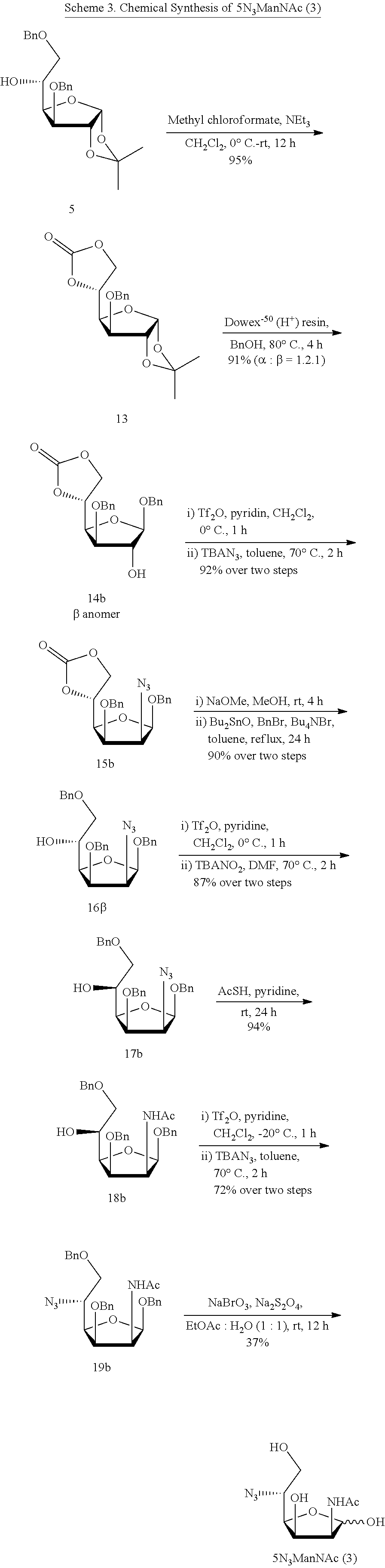

Scheme 3 shows the chemical synthesis of 5N3ManNAc (3) from the intermediate 5 produced from D-glucose.

To synthesize 5N3ManNAc (3), the intermediate diol 5 was treated with methyl chloroformate to produce carbonate 13 in 95% yield (Scheme 3). Opening the isopropylidene ring of 13 with benzyl alcohol in the presence of an acidic cation exchange resin produced benzyl furanoside 14 as an α/β mixture (1.2:1) in 91% yield. The carbonate component was stable under these conditions. The purified B-anomer 14 was used for triflation of the C2—OH which was then subjected to nucleophilic displacement by an azido group to form 2-azido-2-deoxy-mannofuranoside 15ß in 92% yield over two steps. Removal of the carbonate group using NaOMe followed by regio-selective benzylation of C6-OH produced compound 16β in 90% yield over two steps. Another round of triflation followed by TBANO₂-mediated-OH inversion at C-5 position produced L-glucose derivative 17β in 87% yield. To access the per-O-benzylated 5N₃ManNAc compound 19B, compound 17β was converted to compound 18β using AcSH in 94% yield, followed by a two-step azido transfer reaction with inversion of the stereochemistry at the C-5 position with 72% yield. The yield of the two-step azido transfer reaction dropped significantly in the presence of the —NHAc moiety. Without being limited by theory, partial triflation of the —NHAc moiety and instability of the triflate ester during the acid work-up and azido transfer reaction were the major causes of the decreased yield. Debenzylation in the presence of sodium bromate and sodium dithionite in ethyl acetate-water biphasic solvent system produced target ManNAc5N₃ (3) in 37% yield (Scheme 3).

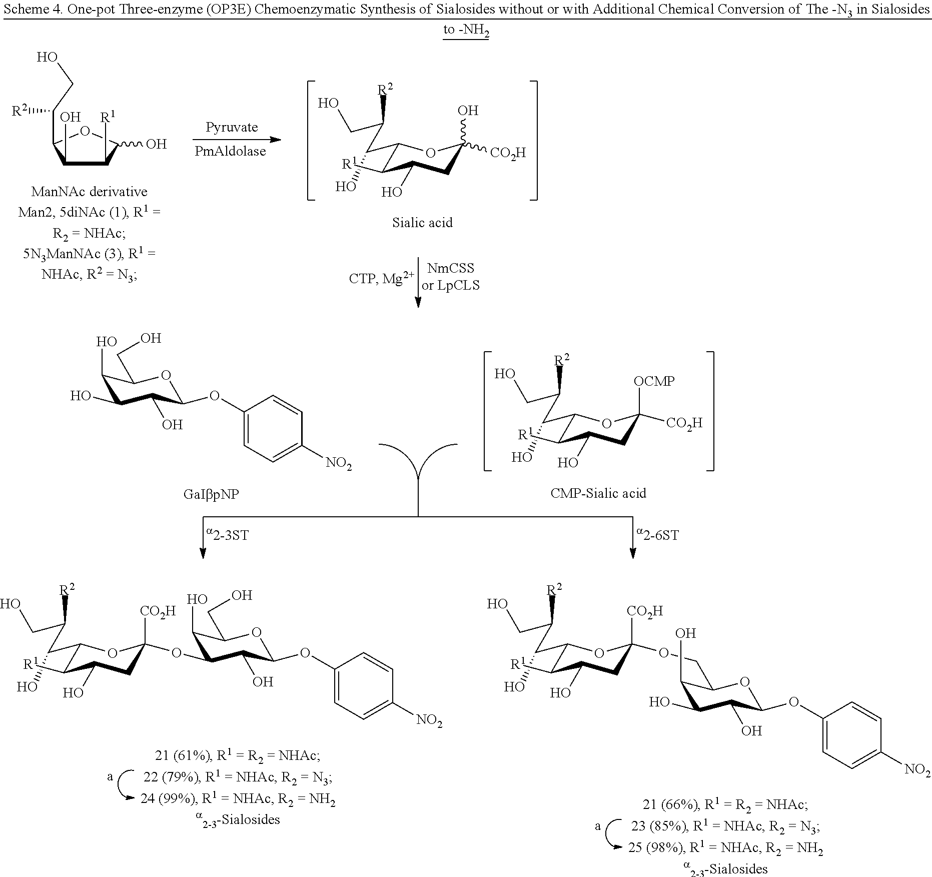

Scheme 4 shows OP3E synthesis of α2-3- and α2-6-linked sialosides using Man2,5diNAc (1), Man2,5diN₃ (2), and 5N₃ManNAc (3) as potential starting materials. Man2,5diNAc (1) and 5N₃ManNAc (3) were successfully used to produce the corresponding 8NAcNeu5Ac- and 8N₃Neu5Ac-containing α2-3/6-linked sialosides, but Man2,5diN₃ (2) was only converted to Neu5,8diN₃, which was not a suitable substrate for NmCSS or LpCLS. Formation of 8NH₂Neu5Ac-sialosides (24-25) were produced from 8N3Neu5Ac-sialosides (22-23) by chemical conversion of the N₃ group of 8N₃Neu5 Ac in the sialosides to an NH₂ group with a) PMe₃, THE/MeOH, 2 N NaOH, r.t., 3 h.

The enzymatic precursor Man2,5diNAc (1) and the chemoenzymatic synthons Man2,5diN3 (2) and 5N₃ManNAc (3) were tested as potential substrates for a recombinant Pasteurella multocida sialic acid aldolase (PmAldolase) together with other enzymes in the sialoside synthetic OP3E systems including Neisseria meningitidis CMP-Neu5Ac synthetase (NmCSS) or Legionella pneumophila CMP-legionaminic acid synthetase (LpCLS), and α2-3- and α2-6-sialyltransferases such as Pasteurella multocida sialyltransferase 1 (PmST1), its mutant PmST1_M144D with reduced α2-3-sialidase and donor hydrolysis activities, and Photobacterium damselae α2-6-sialyltransferase (Pd2,6ST) (Scheme 4). In these OP3E systems, PmAldolase catalyzes the conversion of the relevant six-carbon monosaccharide substrates in the presence of sodium pyruvate to the corresponding nine-carbon sialic acids and derivatives. The sialic acids were then activated by NmCSS or LpCLS in the presence of cytidine 5′-triphosphate (CTP) and magnesium (Mg²⁺) cation to the corresponding CMP-sialic acids and derivatives, which function as donor substrates for the α2-3- and α2-6-sialyltransferases to form the corresponding α2-3- and α2-6-linked sialosides, respectively, using GalβpNP as an acceptor substrate. Man2,5diNAc (1) and 5N₃ManNAc (3) were both suitable starting materials in the NmCSS-containing OP3E systems for the synthesis of the corresponding 8NAcNeu5Ac- and 8N₃Neu5Ac-containing α2-3/6-linked sialosides, respectively, but Man2,5diN3 (2) was not. Stepwise analysis showed that Man2,5diN₃ (2) was tolerated by PmAldolase, the first enzyme in the OP3E, for the formation of Neu5,8diN₃. This sialic acid derivative, however, was not a suitable substrate for either NmCSS or LpCLS to form CMP-Neu5,8diN₃.

The OP3E systems containing PmAldolase, NmCSS, and PmST1 or Pd2,6ST produced 8NAcNeu5Acα2-3GalβpNP (20) and 8NAcNeu5 Acα2-6GalβpNP (21) in 61% and 66% yields, respectively, from the enzymatic precursor Man2,5diNAc (1) and GalβpNP acceptor. Similarly, sialosides 8N₃Neu5Acα2-3GalβpNP (22) and 8N₃Neu5Acα2-6GalβpNP (23) were produced in 79% and 85% yields, respectively, using OP3E systems containing PmAldolase, NmCSS, and PmST1_M144D or Pd2,6ST from 5N3ManNAc (3) and GalβpNP acceptor. Small-scale (10 μL total reaction volume) tests showed that PmST1 was a preferred α2-3-sialyltransferase for synthesizing 8NAcNeu5Acα2-3GalβpNP (20) as PmST1_M144D in the OP3E resulted in a lower yield (~30%). In contrast, PmST1_M144D with a lower sialidase activity was preferred for OP3E synthesis of 8N₃Neu5Acα2-3GalβpNP (22) where the product could be cleaved by the α2-3-sialidase activity of the PmST1 in the presence of sialyltransferase byproduct CMP. The 8-azido group of the sialic acid in sialosides 22-23 obtained was converted to an amino group to obtain sialosides (24-25) containing an 8-NH₂ group in 98-99% yields using the Staudinger reaction in the presence of trimethylphosphine and sodium hydroxide (NaOH, 2 N) in methanol/tetrahydrofuran (THF).

Although the foregoing has been described in some detail by way of illustration and example for purposes of clarity and understanding, one of skill in the art will appreciate that certain changes and modifications can be practiced within the scope of the appended claims. All publications, patents, patent applications, and sequence accession numbers cited herein are hereby incorporated by reference in their entirety for all purposes.

SEQUENCES
SEQ ID NO: 1:
EKQNIAVILARQNSKGLPLKNLRKMNGISLLGHTINAAISSKCFD
RIIVSTDGGLIAEEAKNFGVEVVLRPAELASDTASSISGVIHALE
TIGSNSGTVTLLQPTSPLRTGAHIREAFSLFDEKIKGSVVSACPM
EHHPLKTLLQINNGEYAPMRHLSDLEQPRQQLPQAFRPNGAIYIN
DTASLIANNCFFIAPTKLYIMSHQDSIDIDTELDLQQAENILHHK
ES
SEQ ID NO: 2:
KTITLYLDPASLPALNQLMDFTQNNEDKTHPRIFGLSRFKIPDNI
ITQYQNIHFVELKDNRPTEALFTILDQYPGNIELNIHLNIAHSVQ
LIRPILAYRFKHLDRVSIQQLNLYDDGSDEYVDLEKEENKDISAE
IKQAEKQLSHYLLTGKIKFDNPTIARYVWQSAFPVKYHFLSTDYF
EKAEFLQPLKEYLAENYQKMDWTAYQQLTPEQQAFYLTLVGFNDE
VKQSLEVQQAKFIFTGTTTWEGNTDVREYYAQQQLNLLNHFTQAE
GDLFIGDHYKIYFKGHPRGGEINDYILNNAKNITNIPANISFEVL
MMTGLLPDKVGGVASSLYFSLPKEKISHIIFTSNKQVKSKEDALN
NPYVKVMRRLGIIDESQVIFWDSLKQL
SEQ ID NO: 3:
NLIICCTPLQVLIAEKIIAKFPHTPFYGVMLSTVSNKKFDFYAKR
LAQQCQGFFSMVQHKDRFNLLKEILYLKRTFSGKHFDQVFVANIN
DLQIQFLLSAIDFNLLNTFDDGTINIVPNSLFYQDDPATLQRKLI
NVLLGNKYSIQSLRALSHTHYTIYKGFKNIIERVEPIELVAADNS
EKVTSAVINVLLGQPVFAEDERNIALAERVIKQFNIHYYLPHPRE
KYRLAQVNYIDTELIFEDYILQQCQTHKYCVYTYFSSAIINIMNK
SDNIEVVALKIDTENPAYDACYDLFDELGVNVIDIRE
SEQ ID NO: 4:
DKFAEHEIPKAVIVAGNGESLSQIDYRLLPKNYDVFRCNQFYFEE
RYFLGNKIKAVFFTPGVFLEQYYTLYHLKRNNEYFVDNVILSSFN
HPTVDLEKSQKIQALFIDVINGYEKYLSKLTAFDVYLRYKELYEN
QRITSGVYMCAVAIAMGYTDIYLTGIDFYQASEENYAFDNKKPNI
IRLLPDFRKEKTLFSYHSKDIDLEALSFLQQHYHVNFYSISPMSP
LSKHFPIPTVEDDCETTEVAPLKENYINDILLVDKLAAALE
SEQ ID NO: 5:
CNNSEENTQSIIKNDINKTIIDEEYVNLEPINQSNISFTKHSWVQ
TCGTQQLLTEQNKESISLSVVAPRLDDDEKYCFDFNGVSNKGEKY
ITKVTLNVVAPSLEVYVDHASLPTLQQLMDIIKSEEENPTAQRYI
AWGRIVPTDEQMKELNITSFALINNHTPADLVQEIVKQAQTKHRL
NVKLSSNTAHSFDNLVPILKELNSFNNVTVTNIDLYDDGSAEYVN
LYNWRDTLNKTDNLKIGKDYLEDVINGINEDTSNTGTSSVYNWQK
LYPANYHFLRKDYLTLEPSLHELRDYIGDSLKQMQWDGFKKFNSK
QQELFLSIVNFDKQKLQNEYNSSNLPNFVFTGTTVWAGNHEREYY
AKQQINVINNAINESSPHYLGNSYDLFFKGHPGGGIINTLIMQNY
PSMVDIPSKISFEVLMMTDMLPDAVAGIASSLYFTIPAEKIKFIV
FTSTETITDRETALRSPLVQVMIKLGIVKEENVLFWA
SEQ ID NO: 6:
CNSDNTSLKETVSSNSADVVETETYQLTPIDAPSSFLSHSWEQTC
GTPILNESDKQAISFDFVAPELKQDEKYCFTFKGITGDHRYITNT
TLTVVAPTLEVYIDHASLPSLQQLIHIIQAKDEYPSNQRFVSWKR
VTVDADNANKLNIHTYPLKGNNTSPEMVAAIDEYAQSKNRLNIEF
YTNTAHVENNLPPIIQPLYNNEKVKISHISLYDDGSSEYVSLYQW
KDTPNKIETLEGEVSLLANYLAGTSPDAPKGMGNRYNWHKLYDTD
YYFLREDYLDVEANLHDLRDYLGSSAKQMPWDEFAKLSDSQQTLF
LDIVGFDKEQLQQQYSQSPLPNFIFTGTTTWAGGETKEYYAQQQV
NVINNAINETSPYYLGKDYDLFFKGHPAGGVINDIILGSFPDMIN
IPAKISFEVLMMTDMLPDTVAGIASSLYFTIPADKVNFIVFTSSD
TITDREEALKSPLVQVMLTLGIVKEKDVLFWA
SEQ ID NO: 7:
KKVIIAGNGPSLKEIDYSRLPNDFDVFRCNQFYFEDKYYLGKKCK
AVFYNPSLFFEQYYTLKHLIQNEYETELIMCSNYNQAHLENENFV
KTFYDYFPDAHLGYDFFKQLKDFNAYFKFHEIYFNQRITSGVYMC
AVAIALGYKEIYLSGIDFYQNGSSYAFDTKQKNLLKLAPNFKNDN
SHYIGHSKNTDIKALEFLEKTYKIKLYCLCPNSLLANFIELAPNL
MSNFIIQEKNNYTKDILIPSSEAYGKFSKNIN
SEQ ID NO: 8:
NELNYGQLSISPIFQGGSYQLNNKSIDISPLLLDKLSGDSQTVVM
KFKADKPNSLQALFGLSNSKAGFKNNYFSIFMRDSGEIGVEIRDA
QKGINYLFSRPASLWGKHKGQAVENTLVFVSDSKDKTYTMYVNGI
EVFSETVDTFLPISNINGIDKATLGAVNREGKEHYLAKGSIDEIS
LENKAISDQEVSTIPLSNPFQLIFQSGDSTQANYFRIPTLYTLSS
GRVLSSIDARYGGTHDSKSKINIATSYSDDNGKTWSEPIFAMKFN
DYEEQLVYWPRDNKLKNSQISGSASFIDSSIVEDKKSGKTILLAD
VMPAGIGNNNANKADSGFKEINGHYYLKLKKNGDNDFRYTVRENG
VVYDETTNKPTNYTINDKYEVLEGGKSLTVEQYSVDFDSGSLRER
HNGKQVPMNVFYKDSLFKVTPTNYIAMTTSQNRGESWEQFKLLPP
FLGEKHNGTYLCPGQGLALKSSNRLIFATYTSGELTYLISDDSGQ
TWKKSSASIPFENATAEAQMVELRDGVIRTFFRTTTGKIAYMTSR
DSGETWSEVSYIDGIQQTSYGTQVSAIKYSQLIDGKEAVILSTPN
SRSGRKGGQLVVGLVNKEDDSIDWKYHYDIDLPSYGYAYSAITEL
PNHHIGVLFEKYDSWSRNELHLSNVVQYIDLEINDLTK
SEQ ID NO: 9:
KKNIKQYVTLGTVVVLSAFVANSVAAQETETSEVSTPELVQPVAP
TTSISEVQHKSGNSSEVTVQPRTVETTVKDPSSTAEETLVLEKNN
VTLTGGGENVTKELKDKFTSGDFTVVIKYNQSSEKGLQALFGISN
SKPGQQNSYVDVFLRDNGELGMEARDTSSNKNNLVSRPASVWGKY
KQEAVTNTVAVVADSVKKTYSLYANGTKVVEKKVDNFLNIKDIKG
IDYYMLGGVKRAGKTAFGFNGTLENIKFFNSALDEETVKKMTTNA
VTGHLIYTANDTTGSNYFRIPVLYTFSNGRVFSSIDARYGGTHDF
LNKINIATSYSDDNGKTWTKPKLTLAFDDFAPVPLEWPREVGGRD
LQISGGATYIDSVIVEKKNKQVLMFADVMPAGVSFREATRKDSGY
KQIDGNYYLKLRKQGDTDYNYTIRENGTVYDDRTNRPTEFSVDKN
FGIKQNGNYLTVEQYSVSFENNKKTEYRNGTKVHMNIFYKDALFK
VVPTNYIAYISSNDHGESWSAPTLLPPIMGLNRNAPYLGPGRGII
ESSTGRILIPSYTGKESAFIYSDDNGASWKVKVVPLPSSWSAEAQ
FVELSPGVIQAYMRTNNGKIAYLTSKDAGTTWSAPEYLKFVSNPS
YGTQLSIINYSQLIDGKKAVILSTPNSTNGRKHGQIWIGLINDDN
TIDWRYHHDVDYSNYGYSYSTLTELPNHEIGLMFEKFDSWSRNEL
HMKNVVPYITFKIEDLKKN
SEQ ID NO: 10:
KKNIKQYVTLGTVVVLSAFVANSVAAQETETSEVSTPKLVQPVAP
TTPISEVQPTSDNSSEVTVQPRTVETTVKDPSSTAEETPVLEKNN
VTLTGGGENVTKELKDKFTSGDFTVVIKYNQSSEKGLQALFGISN
SKPGQQNSYVDVFLRDNGELGMEARDTSSNKNNLVSRPASVWGKY
KQEAVTNTVAVVADSVKKTYSLYANGTKVVEKKVDNFLNIKDIKG
IDYYMLGGVKRAGKTAFGFNGTLENIKFFNSALDEETVKKMTTNA
VTGHLIYTANDTTGSNYFRIPVLYTFSNGRVFSSIDARYGGTHDF
LNKINIATSYSDDNGKTWTKPKLTLAFDDFAPVPLEWPREVGGRD
LQISGGATYIDSVIVEKKNKQVLMFADVMPAGVSFREATRKDSGY
KQIDGNYYLKLRKQGDTDYNYTIRENGTVYDDRTNRPTEFSVDKN
FGIKQNGNYLTVEQYSVSFENNKKTEYRNGTKVHMNIFYKDALFK
VVPTNYIAYISSNDHGESWSAPTLLPPIMGLNRNAPYLGPGRGII
ESSTGRILIPSYTGKESAFIYSDDNGASWKVKVVPLPSSWSAEAQ
FVELSPGVIQAYMRTNNGKIAYLTSKDAGTTWSAPEYLKFVSNPS
YGTQLSIINYSQLIDGKKAVILSTPNSTNGRKHGQIWIGLINDDN
TIDWRYHHDVDYSNYGYSYSTLTELPNHEIGLMFEKFDSWSRNEL
HMKNVVPYITFKIEDLKKN
SEQ ID NO: 11:
TNIAIIPARAGSKGIPDKNLQPVGGHSLIGRAILAAKNADVFDMI
VVTSDGDNILREAEKYGALALKRPAELAQDNSRTIDAILHALESL
NIREGTCTLLQPTSPLRDHLDIKNAMDMYVNGGVHSVVSACECEH
HPYKAFALSKDHEVLPVREIADFEAVRQTLPKMYRANGAIYINDI
AQLLKEKYFFIPPLKFYLMPTYHSVDIDVKQDLELAEILSNK

### REFERENCES

1. Chen, X.; Varki, A., Advances in the Biology and Chemistry of Sialic Acids. ACS Chemical Biology 2010, 5 (2), 163-176.
2. Varki, A., Glycan-based interactions involving vertebrate sialic-acid-recognizing proteins. Nature 2007, 446 (7139), 1023-1029.
3. Martin, L. T.; Marth, J. D.; Varki, A.; Varki, N. M., Genetically Altered Mice with Different Sialyltransferase Deficiencies Show Tissue-specific Alterations in Sialylation and Sialic Acid 9-O-Acetylation. Journal of Biological Chemistry 2002, 277 (36), 32930-32938.
4. Wasik, B. R.; Barnard, K. N.; Ossiboff, R. J.; Khedri, Z.; Feng, K. H.; Yu, H.; Chen, X.; Perez, D. R.; Varki, A.; Parrish, C. R., Distribution of O-Acetylated Sialic Acids among Target Host Tissues for Influenza Virus. mSphere 2017, 2 (5).
5. Regl, G.; Kaser, A.; Iwersen, M.; Schmid, H.; Kohla, G.; Strobl, B.; Vilas, U.; Schauer, R.; Vlasak, R., The hemagglutinin-esterase of mouse hepatitis virus strain S is a sialate-4-O-acetylesterase. Journal of virology 1999, 73 (6), 4721-7.
6. Klein, A.; Roussel, P., O-Acetylation of sialic acids. Biochimie 1998, 80 (1), 49-57.
7. Yu, H.; Zeng, J.; Li, Y.; Thon, V.; Shi, B.; Chen, X., Effective one-pot multienzyme (OPME) synthesis of monotreme milk oligosaccharides and other sialosides containing 4-O-acetyl sialic acid. Org Biomol Chem 2016, 14 (36), 8586-97.
8. Taylor, G., Sialidases: structures, biological significance and therapeutic potential. Current Opinion in Structural Biology 1996, 6 (6), 830-837.
9. Corfield, T., Bacterial sialidases-roles in pathogenicity and nutrition. Glycobiology 1992, 2 (6), 509-521.
10. von Itzstein, M., The war against influenza: discovery and development of sialidase inhibitors. Nature Reviews Drug Discovery 2007, 6 (12), 967-974.
11. Moscona, A., Entry of parainfluenza virus into cells as a target for interrupting childhood respiratory disease. The Journal of clinical investigation 2005, 115 (7), 1688-1698.
12. Schauer, R., Achievements and challenges of sialic acid research. Glycoconjugate Journal 2000, 17 (7), 485-499.
13. Varki, A., Diversity in the sialic acids. Glycobiology 1992, 2 (1), 25-40.
14. Angata, T.; Varki, A., Chemical Diversity in the Sialic Acids and Related a-Keto Acids: An Evolutionary Perspective. Chemical Reviews 2002, 102 (2), 439-470.
15. Yu, H.; Zeng, J.; Li, Y.; Thon, V.; Shi, B.; Chen, X., Effective one-pot multienzyme (OPME) synthesis of monotreme milk oligosaccharides and other sialosides containing 4-O-acetyl sialic acid. Organic & Biomolecular Chemistry 2016, 14 (36), 8586-8597.
16. Khedri, Z.; Xiao, A.; Yu, H.; Landig, C. S.; Li, W.; Diaz, S.; Wasik, B. R.; Parrish, C. R.; Wang, L.-P.; Varki, A.; Chen, X., A Chemical Biology Solution to Problems with Studying Biologically Important but Unstable 9-O-Acetyl Sialic Acids. ACS Chem. Biol. 2017, 12 (1), 214-224.
17. Li, W.; Xiao, A.; Li, Y.; Yu, H.; Chen, X., Chemoenzymatic synthesis of Neu5Ac9NAc-containing α2-3- and α2-6-linked sialosides and their use for sialidase substrate specificity studies. Carbohydrate research 2017, 451, 51-58.
18. Ferraris, O.; Lina, B., Mutations of neuraminidase implicated in neuraminidase inhibitors resistance. J Clin Virol 2008, 41 (1), 13-9.
19. Li, J.; Chen, Y.; Yuan, N.; Zeng, M.; Zhao, Y.; Yu, R.; Liu, Z.; Wu, H.; Dong, S., A Novel Natural Influenza A HINI Virus Neuraminidase Inhibitory Peptide Derived from Cod Skin Hydrolysates and Its Antiviral Mechanism. Mar Drugs 2018, 16 (10).
20. Lin, D.; Yi, Y. J.; Xiao, M. W.; Chen, J.; Ye, J.; Hu, A. X.; Lian, W. W.; Liu, A. L.; Du, G. H., Design, synthesis and biological evaluation of honokiol derivatives as influenza neuraminidase inhibitors. J Asian Nat Prod Res 2019, 21 (11), 1052-1067.
21. Kim, B. R.; Park, J.-Y.; Jeong, H. J.; Kwon, H.-J.; Park, S.-J.; Lee, I.-C.; Ryu, Y. B.; Lee, W. S., Design, synthesis, and evaluation of curcumin analogues as potential inhibitors of bacterial sialidase. Journal of enzyme inhibition and medicinal chemistry 2018, 33 (1), 1256-1265.
22. Choi, H. M.; Kim, J. Y.; Li, Z. P.; Jenis, J.; Ban, Y. J.; Baiseitova, A.; Park, K. H., Effectiveness of Prenyl Group on Flavonoids from Epimedium koreanum Nakai on Bacterial Neuraminidase Inhibition. Molecules 2019, 24 (2).
23. Kim, J.-H.; Resende, R.; Wennekes, T.; Chen, H.-M.; Bance, N.; Buchini, S.; Watts, A. G.; Pilling, P.; Streltsov, V. A.; Petric, M.; Liggins, R.; Barrett, S.; McKimm-Breschkin, J. L.; Niikura, M.; Withers, S. G., Mechanism-Based Covalent Neuraminidase Inhibitors with Broad-Spectrum Influenza Antiviral Activity. Science 2013, 340 (6128), 71.
24. Buchini, S.; Buschiazzo, A.; Withers, S. G., A New Generation of Specific Trypanosoma cruzi trans-Sialidase Inhibitors. Angewandte Chemie International Edition 2008, 47 (14), 2700-2703.
25. Buchini, S.; Gallat, F. X.; Greig, I. R.; Kim, J. H.; Wakatsuki, S.; Chavas, L. M.; Withers, S. G., Tuning mechanism-based inactivators of neuraminidases: mechanistic and structural insights. Angew Chem Int Ed Engl 2014, 53 (13), 3382-6.
26. Withers, S. G.; Rupitz, K.; Street, I. P., 2-Deoxy-2-fluoro-D-glycosyl fluorides. A new class of specific mechanism-based glycosidase inhibitors. J Biol Chem 1988, 263 (17), 7929-32.
27. Li, W.; Santra, A.; Yu, H.; Slack, T. J.; Muthana, M. M.; Shi, D.; Liu, Y.; Chen, X., 9-Azido-9-deoxy-2,3-difluorosialic Acid as a Subnanomolar Inhibitor against Bacterial Sialidases. The Journal of Organic Chemistry 2019, 84 (11), 6697-6708.
28. McKimm-Breschkin, J. L.; Barrett, S.; Pilling, P. A.; Hader, S.; Watts, A. G.; Streltsov, V. A., Structural and Functional Analysis of Anti-Influenza Activity of 4-, 7-, 8- and 9-Deoxygenated 2,3-Difluoro-N-acetylneuraminic Acid Derivatives. J. Med. Chem. 2018, 61 (5), 1921-1933.
29. Tai, S. H.; Agafitei, O.; Gao, Z.; Liggins, R.; Petric, M.; Withers, S. G.; Niikura, M., Difluorosialic acids, potent novel influenza virus neuraminidase inhibitors, induce fewer drug resistance-associated neuraminidase mutations than does oseltamivir. Virus Res 2015, 210, 126-32.
30. Rudrawar, S.; Dyason, J. C.; Rameix-Welti, M. A.; Rose, F. J.; Kerry, P. S.; Russell, R. J.; van der Werf, S.; Thomson, R. J.; Naffakh, N.; von Itzstein, M., Novel sialic acid derivatives lock open the 150-loop of an influenza A virus group-1 sialidase. Nat Commun 2010, 1, 113.
31. Colombo, C.; Pinto, B. M.; Bernardi, A.; Bennet, A. J., Synthesis and evaluation of influenza A viral neuraminidase candidate inhibitors based on a bicyclo[3.1.0]hexane scaffold. Organic & Biomolecular Chemistry 2016, 14 (27), 6539-6553.
32. Russell, R. J.; Haire, L. F.; Stevens, D. J.; Collins, P. J.; Lin, Y. P.; Blackburn, G. M.; Hay, A. J.; Gamblin, S. J.; Skehel, J. J., The structure of H₅N1 avian influenza neuraminidase suggests new opportunities for drug design. Nature 2006, 443 (7107), 45-49.
33. Winger, M.; von Itzstein, M., Exposing the Flexibility of Human Parainfluenza Virus Hemagglutinin-neuraminidase. J. Am. Chem. Soc. 2012, 134 (44), 18447-18452.
34. Chibanga, V. P.; Dirr, L.; Guillon, P.; El-Deeb, I. M.; Bailly, B.; Thomson, R. J.; von Itzstein, M., New antiviral approaches for human parainfluenza: Inhibiting the haemagglutinin-neuraminidase. Antiviral Res 2019, 167, 89-97.
35. Dirr, L.; El-Deeb, I. M.; Chavas, L. M. G.; Guillon, P.; Itzstein, M. v., The impact of the butterfly effect on human parainfluenza virus haemagglutinin-neuraminidase inhibitor design. Scientific reports 2017, 7 (1), 4507-4507.
36. Xiao, A.; Slack, T. J.; Li, Y.; Shi, D.; Yu, H.; Li, W.; Liu, Y.; Chen, X., Streptococcus pneumoniae Sialidase SpNanB-Catalyzed One-Pot Multienzyme (OPME) Synthesis of 2,7-Anhydro-Sialic Acids as Selective Sialidase Inhibitors. J. Org. Chem. 2018, 83 (18), 10798-10804.
37. Xiao; Li; Li; Santra; Yu; Li; Chen, Sialidase-catalyzed one-pot multienzyme (OPME) synthesis of sialidase transition-state analogue inhibitors. ACS catalysis 2018, 8 (1), 43-47.
38. Holzer, C. T.; Von Itzstein, M.; Jin, B.; Pegg, M. S.; Stewart, W. P.; Wu, W.-Y., Inhibition of sialidases from viral, bacterial and mammalian sources by analogues of 2-deoxy-2,3-didehydro-N-acetylneuraminic acid modified at the C-4 position. Glycoconjugate Journal 1993, 10 (1), 40-44.
39. Meindl, P.; Bodo, G.; Palese, P.; Schulman, J.; Tuppy, H., Inhibition of neuraminidase activity by derivatives of 2-deoxy-2,3-dehydro-N-acetylneuraminic acid. Virology 1974, 58 (2), 457-463.
40. Assailly, C.; Bridot, C.; Saumonneau, A.; Lottin, P.; Roubinet, B.; Krammer, E.-M.; François, F.; Vena, F.; Landemarre, L.; Alvarez Dorta, D.; Deniaud, D.; Grandjean, C.; Tellier, C.; Pascual, S.; Montembault, V.; Fontaine, L.; Daligault, F.; Bouckaert, J.; Gouin, S. G., Polyvalent Transition-State Analogues of Sialyl Substrates Strongly Inhibit Bacterial Sialidases **. Chemistry—A European Journal 2021, 27 (9), 3142-3150.
41. Slack, T. J.; Li, W.; Shi, D.; McArthur, J. B.; Zhao, G.; Li, Y.; Xiao, A.; Khedri, Z.; Yu, H.; Liu, Y.; Chen, X., Triazole-linked transition state analogs as selective inhibitors against V. cholerae sialidase. Bioorganic & medicinal chemistry 2018, 26 (21), 5751-5757.
42. Hyun, S. W.; Liu, A.; Liu, Z.; Cross, A. S.; Verceles, A. C.; Magesh, S.; Kommagalla, Y.; Kona, C.; Ando, H.; Luzina, I. G.; Atamas, S. P.; Piepenbrink, K. H.; Sundberg, E. J.; Guang, W.; Ishida, H.; Lillehoj, E. P.; Goldblum, S. E., The NEU1-selective sialidase inhibitor, C₉-butyl-amide-DANA, blocks sialidase activity and NEU1-mediated bioactivities in human lung in vitro and murine lung in vivo. Glycobiology 2016, 26 (8), 834-49.
43. Laborda, P.; Wang, S. Y.; Voglmeir, J., Influenza Neuraminidase Inhibitors: Synthetic Approaches, Derivatives and Biological Activity. Molecules 2016, 21 (11).
44. Kim, C. U.; Lew, W.; Williams, M. A.; Liu, H.; Zhang, L.; Swaminathan, S.; Bischofberger, N.; Chen, M. S.; Mendel, D. B.; Tai, C. Y.; Laver, W. G.; Stevens, R. C., Influenza Neuraminidase Inhibitors Possessing a Novel Hydrophobic Interaction in the Enzyme Active Site: Design, Synthesis, and Structural Analysis of Carbocyclic Sialic Acid Analogues with Potent Anti-Influenza Activity. Journal of the American Chemical Society 1997, 119 (4), 681-690.
45. von Itzstein, M.; Wu, W.-Y.; Kok, G. B.; Pegg, M. S.; Dyason, J. C.; Jin, B.; Van Phan, T.; Smythe, M. L.; White, H. F.; Oliver, S. W.; Colman, P. M.; Varghese, J. N.; Ryan, D. M.; Woods, J. M.; Bethell, R. C.; Hotham, V. J.; Cameron, J. M.; Penn, C. R., Rational design of potent sialidase-based inhibitors of influenza virus replication. Nature 1993, 363 (6428), 418-423.
46. Li, Z.; Unione, L.; Liu, L.; Lang, Y.; de Vries, R. P.; de Groot, R. J.; Boons, G.-J., Synthetic O-Acetylated Sialosides and their Acetamido-deoxy Analogues as Probes for Coronaviral Hemagglutinin-esterase Recognition. J. Amer. Chem. Soc. 2022, 144 (1), 424-435.
47. Lu, Y.; Gervay-Hague, J., Synthesis of C-4 and C-7 triazole analogs of zanamivir as multivalent sialic acid containing scaffolds. Carbohydrate research 2007, 342 (12-13), 1636-50.
48. Ciccotosto, S.; von Itzstein, M., Synthesis of methyl 5-acetamido-3,4,5-trideoxy-4-guanidinyl-d-glycero-d-galacto-2-nonulopyranosidonic acid (4-deoxy-4-guanidino-Neu5Acα2Me). Tetrahedron Letters 1995, 36 (30), 5405-5408.
49. Yu, H.; Chen, X., One-pot multienzyme (OPME) systems for chemoenzymatic synthesis of carbohydrates. Organic & Biomolecular Chemistry 2016, 14 (10), 2809-2818.
50. Yu, H.; Yu, H.; Karpel, R.; Chen, X., Chemoenzymatic synthesis of CMP-sialic acid derivatives by a one-pot two-enzyme system: comparison of substrate flexibility of three microbial CMP-sialic acid synthetases. Bioorganic & Medicinal Chemistry 2004, 12 (24), 6427-6435.
51. Yu, H.; Chokhawala, H.; Karpel, R.; Yu, H.; Wu, B.; Zhang, J.; Zhang, Y.; Jia, Q.; Chen, X., A Multifunctional Pasteurella multocida Sialyltransferase: A Powerful Tool for the Synthesis of Sialoside Libraries. Journal of the American Chemical Society 2005, 127 (50), 17618-17619.
52. Ding, L.; Zhao, C.; Qu, J.; Li, Y.; Sugiarto, G.; Yu, H.; Wang, J.; Chen, X., A Photobacterium sp. α2-6-sialyltransferase (Psp2,6ST) mutant with an increased expression level and improved activities in sialylating Tn antigens. Carbohydrate Research 2015, 408, 127-133.
53. Ding, L.; Yu, H.; Lau, K.; Li, Y.; Muthana, S.; Wang, J.; Chen, X., Efficient chemoenzymatic synthesis of sialyl Tn-antigens and derivatives. Chemical Communications 2011, 47 (30), 8691-8693.
54. Chokhawala.; Yu, Chen, High-Throughput Substrate Specificity Studies of Sialidases by Using Chemoenzymatically Synthesized Sialoside Libraries. ChemBioChem 2007, 8 (2), 194-201.
55. Yu, H.; Yu, H.; Karpel, R.; Chen, X. Chemoenzymatic synthesis of CMP-sialic acid derivatives by a one-pot two-enzyme system: comparison of substrate flexibility of three microbial CMP-sialic acid synthetases. Bioorg. Med. Chem. 2004, 12, 6427-6435.
56. Yu, H.; Chokhawala, H.; Karpel, R.; Yu, H.; Wu, B.; Zhang, J.; Zhang, Y.; Jia, Q.; Chen, X. A multifunctional Pasteurella multocida sialyltransferase: a powerful tool for the synthesis of sialoside libraries. J. Am. Chem. Soc. 2005, 127, 17618-17619.
57. Sugiarto, G.; Lau, K.; Qu, J.; Li, Y.; Lim, S.; Mu, S.; Ames, J. B.; Fisher, A. J.; Chen, X. A sialyltransferase mutant with decreased donor hydrolysis and reduced sialidase activities for directly sialylating LewisX. ACS Chem. Biol. 2012, 7, 1232-1240.
58. Zhang, L.; Li, Y.; Li, R.; Yang, X.; Zheng, Z.; Fu, J.; Yu, H.; Chen, X. Glycoprotein in vitro N-glycan processing using enzymes expressed in E. coli. Molecules 2023, 28, 2753.
59. Li, Y.; Cao, H.; Yu, H.; Chen, Y.; Lau, K.; Qu, J.; Thon, V.; Sugiarto, G.; Chen, X. Identifying selective inhibitors against the human cytosolic sialidase NEU2 by substrate specificity studies. Mol. Biosyst. 2011, 7, 1060-1072.
60. Tasnima, N.; Yu, H.; Li, Y.; Santra, A.; Chen, X. Chemoenzymatic synthesis of para-nitrophenol (pNP)-tagged alpha2-8-sialosides and high-throughput substrate specificity studies of alpha2-8-sialidases. Org. Biomol. Chem. 2016, 15, 160-167.
61. Xiao, A.; Slack, T. J.; Li, Y.; Shi, D.; Yu, H.; Li, W.; Liu, Y.; Chen, X. Streptococcus pneumoniae sialidase SpNanB-catalyzed one-pot multienzyme (OPME) synthesis of 2,7-anhydro-sialic acids as selective sialidase inhibitors. J. Org. Chem. 2018, 83, 10798-10804.
62. Xiao; Li.; Li; Santra, A.; Yu, H.; Li, W.; Chen, X. Sialidase-catalyzed one-pot multienzyme (OPME) synthesis of sialidase transition-state analogue inhibitors. ACS Catal. 2018, 8, 43-47.
63. Sela, D. A.; Li, Y.; Lerno, L.; Wu, S.; Marcobal, A. M.; German, J. B.; Chen, X.; Lebrilla, C. B.; Mills, D. A. An infant-associated bacterial commensal utilizes breast milk sialyloligosaccharides. J. Biol. Chem. 2011, 286, 11909-11918.
64. Mishra, B.; Yuan, Y.; Yu, H.; Kang, H.; Gao, J.; Daniels, R.; Chen, X. Synthetic sialosides terminated with 8-N-substituted sialic acid as selective substrates for sialidases from bacteria and influenza viruses. Angew. Chem. Int. Ed. Engl. 2024, 63, e202403133.
65. Lau, K.; Thon, V.; Yu, H.; Ding, L.; Chen, Y.; Muthana, M. M.; Wong, D.; Huang, R.; Chen, X. Highly efficient chemoenzymatic synthesis of betal-4-linked galactosides with promiscuous bacterial beta1-4-galactosyltransferases. Chem. Commun. 2010, 46, 6066-6068.
66. Xiong, Y.; Hirano, H.; Lane, N. E.; Nandi, S.; McDonald, K. A. Plant-based production and characterization of a promising Fc-fusion protein against microgravity-induced bone density loss. Front. Bioeng. Biotechnol. 2022, 10, 962292.
67. Taubenberger, J. K.; Morens, D. M. Influenza: the once and future pandemic. Public Health Rep. 2010, 125 Suppl 3 (Suppl 3), 16-26.
68. Sautto, G. A.; Kirchenbaum, G. A.; Ross, T. M. Towards a universal influenza vaccine: different approaches for one goal. Virol. J. 2018, 15, 17.
69. Kanaujia, R.; Bora, I.; Ratho, R. K.; Thakur, V.; Mohi, G. K.; Thakur, P. Avian influenza revisited: concerns and constraints. Virusdisease 2022, 33, 456-465.
70. Morens, D. M.; Park, J.; Taubenberger, J. K. Many potential pathways to future pandemic influenza. Sci. Transl. Med. 2023, 15, eadj2379.
71. Noshi, T.; Kitano, M.; Taniguchi, K.; Yamamoto, A.; Omoto, S.; Baba, K.; Hashimoto, T.; Ishida, K.; Kushima, Y.; Hattori, K.; Kawai, M.; Yoshida, R.; Kobayashi, M.; Yoshinaga, T.; Sato, A.; Okamatsu, M.; Sakoda, Y.; Kida, H.; Shishido, T.; Naito, A. In vitro characterization of baloxavir acid, a first-in-class cap-dependent endonuclease inhibitor of the influenza virus polymerase PA subunit. Antiviral Res. 2018, 160, 109-117.
72. Li, Y.; Huo, S.; Yin, Z.; Tian, Z.; Huang, F.; Liu, P.; Liu, Y.; Yu, F. Retracted and republished from: “The current state of research on influenza antiviral drug development: drugs in clinical trial and licensed drugs”. mBio 2024, 15, e0017524.
73. Wu, N. C.; Wilson, I. A. Influenza Hemagglutinin Structures and Antibody Recognition. Cold Spring Harb Perspect Med 2020, 10 (8).
74. Harris, A.; Cardone, G.; Winkler, D. C.; Heymann, J. B.; Brecher, M.; White, J. M.; Steven, A. C. Influenza virus pleiomorphy characterized by cryoelectron tomography. Proc. Natl. Acad. Sci. U.S.A 2006, 103, 19123-19127.
75. Erbelding, E. J.; Post, D. J.; Stemmy, E. J.; Roberts, P. C.; Augustine, A. D.; Ferguson, S.; Paules, C. I.; Graham, B. S.; Fauci, A. S. A universal influenza vaccine: The strategic plan for the National Institute of Allergy and Infectious Diseases. J. Infect. Dis. 2018, 218, 347-354.
76. Lim, C. M. L.; Komarasamy, T. V.; Adnan, N.; Radhakrishnan, A. K.; Balasubramaniam, V. Recent advances, approaches and challenges in the development of universal influenza vaccines. Influenza Other Respir. Viruses 2024, 18, e13276.
77. Krammer, F.; Fouchier, R. A. M.; Eichelberger, M. C.; Webby, R. J.; Shaw-Saliba, K.; Wan, H.; Wilson, P. C.; Compans, R. W.; Skountzou, I.; Monto, A. S. How can neuraminidase-based immunity contribute to better influenza virus vaccines? mBio 2018, 9, e02332-17.
78. Monto, A. S.; Petrie, J. G.; Cross, R. T.; Johnson, E.; Liu, M.; Zhong, W.; Levine, M.; Katz, J. M.; Ohmit, S. E. Antibody to influenza virus neuraminidase: An independent correlate of protection. J. Infect. Dis. 2015, 212, 1191-1199.
79. Maier, H. E.; Nachbagauer, R.; Kuan, G.; Ng, S.; Lopez, R.; Sanchez, N.; Stadlbauer, D.; Gresh, L.; Schiller, A.; Rajabhathor, A.; Ojeda, S.; Guglia, A. F.; Amanat, F.; Balmaseda, A.; Krammer, F.; Gordon, A. Pre-existing antineuraminidase antibodies are associated with shortened duration of influenza A (H1N1) pdm virus shedding and illness in naturally infected adults. Clin. Infect. Dis. 2020, 70, 2290-2297.
80. Couch, R. B.; Atmar, R. L.; Franco, L. M.; Quarles, J. M.; Wells, J.; Arden, N.; Nino, D.; Belmont, J. W. Antibody correlates and predictors of immunity to naturally occurring influenza in humans and the importance of antibody to the neuraminidase. J. Infect. Dis. 2013, 207, 974-981.
81. Memoli, M. J.; Shaw, P. A.; Han, A.; Czajkowski, L.; Reed, S.; Athota, R.; Bristol, T.; Fargis, S.; Risos, K.; Powers, J. H.; Davey, R. T. Jr.; Taubenberger, J. K. Evaluation of antihemagglutinin and antineuraminidase antibodies as correlates of protection in an influenza A/H1N1 virus healthy human challenge model. mBio 2016, 7, e00417-16.
82. Wilson, J. R.; Guo, Z.; Reber, A.; Kamal, R. P.; Music, N.; Gansebom, S.; Bai, Y.; Levine, M.; Carney, P.; Tzeng, W. P.; Stevens, J.; York, I. A. An influenza A virus (H₇N9) anti-neuraminidase monoclonal antibody with prophylactic and therapeutic activity in vivo. Antiviral Res. 2016, 135, 48-55.
83. Stadlbauer, D.; Zhu, X.; McMahon, M.; Turner, J. S.; Wohlbold, T. J.; Schmitz, A. J.; Strohmeier, S.; Yu, W.; Nachbagauer, R.; Mudd, P. A.; Wilson, I. A.; Ellebedy, A. H.; Krammer, F. Broadly protective human antibodies that target the active site of influenza virus neuraminidase. Science 2019, 366, 499-504.
84. Krammer, F.; Weir, J. P.; Engelhardt, O.; Katz, J. M.; Cox, R. J. Meeting report and review: Immunological assays and correlates of protection for next-generation influenza vaccines. Influenza Other Respir. Viruses 2020, 14, 237-243.
85. Abed; Hardy.; Li; Boivin. Divergent evolution of hemagglutinin and neuraminidase genes in recent influenza A: H3N2 viruses isolated in Canada. J Med Virol 2002, 67, 589-595.
86. Peteranderl, C.; Herold, S.; Schmoldt, C. Human influenza virus infections. Semin. Respir. Crit. Care Med. 2016, 37, 487-500.
87. Lampejo, T. Influenza and antiviral resistance: an overview. Eur. J. Clin. Microbiol. Infect. Dis. 2020, 39, 1201-1208.
88. Kim, H. M.; Lee, N.; Kim, M. S.; Kang, C.; Chung, Y. S. Characterization of neuraminidase inhibitor-resistant influenza virus isolates from immunocompromised patients in the Republic of Korea. Virol. J. 2020, 17, 94.
89. Chong, Y.; Matsumoto, S.; Kang, D.; Ikematsu, H. Consecutive influenza surveillance of neuraminidase mutations and neuraminidase inhibitor resistance in Japan. Influenza Other Respir. Viruses 2019, 13, 115-122.
90. Ferraris, O.; Lina, B. Mutations of neuraminidase implicated in neuraminidase inhibitors resistance. J. Clin. Virol. 2008, 41, 13-19.
91. Pinsky, B. A.; Bradley, B. T. Opportunities and challenges for the U. S. laboratory response to highly pathogenic avian influenza A (H5N1). J. Clin. Virol. 2024, 174, 105723.
92. Wang, Y.; Chen, J.; Zhang, Y.; Yang, Z.; Zhang, K.; Zhang, D.; Zheng, L. Advancing microfluidic immunity testing systems: New trends for microbial pathogen detection. Molecules 2024, 29, 3322.
93. Peng, B.; Wang, Y.; Xie, Y.; Dong, X.; Liu, W.; Li, D.; Chen, H. An overview of influenza A virus detection methods: from state-of-the-art of laboratories to point-of-care strategies. Anal. Methods 2024, 16, 4496-4515.
94. Chokhawala; Yu; Chen. High-throughput substrate specificity studies of sialidases by using chemoenzymatically synthesized sialoside libraries. Chembiochem 2007, 8, 194-201.
95. Li, Y.; Cao, H.; Dao, N.; Luo, Z.; Yu, H.; Chen, Y.; Xing, Z.; Baumgarth, N.; Cardona, C.; Chen, X. High-throughput neuraminidase substrate specificity study of human and avian influenza A viruses. Virology 2011, 415, 12-19.
96. Kooner, A. S.; Yuan, Y.; Yu, H.; Kang, H.; Klenow, L.; Daniels, R.; Chen, X. Sialosides Containing 7-N-Acetyl Sialic Acid Are Selective Substrates for Neuraminidases from Influenza A Viruses. ACS Infect. Dis. 2023, 9, 33-41.
97. Mishra, B.; Yuan, Y.; Yu, H.; Kang, H.; Gao, J.; Daniels, R.; Chen, X. Synthetic Sialosides Terminated with 8-N-Substituted Sialic Acid as Selective Substrates for Sialidases from Bacteria and Influenza Viruses. Angew. Chem. Int. Ed. Engl. 2024, 63, e202403133.
98. von Itzstein, M. The war against influenza: discovery and development of sialidase inhibitors. Nat. Rev. Drug Discov. 2007, 6, 967-974.
99. von Itzstein, M.; Wu, W. Y.; Kok, G. B.; Pegg, M. S.; Dyason, J. C.; Jin, B.; Van Phan, T.; Smythe, M. L.; White, H. F.; Oliver, S. W.; et al. Rational design of potent sialidase-based inhibitors of influenza virus replication. Nature 1993, 363, 418-423.
100. Li, W.; Escarpe, P. A.; Eisenberg, E. J.; Cundy, K. C.; Sweet, C.; Jakeman, K. J.; Merson, J.; Lew, W.; Williams, M.; Zhang, L.; Kim, C. U.; Bischofberger, N.; Chen, M. S.; Mendel, D. B. Identification of GS 4104 as an orally bioavailable prodrug of the influenza virus neuraminidase inhibitor GS 4071. Antimicrob. Agents Chemother. 1998, 42, 647-653.
101. Holzer, C. T.; von Itzstein, M.; Jin, B.; Pegg, M. S.; Stewart, W. P.; Wu, W. Y. Inhibition of sialidases from viral, bacterial and mammalian sources by analogues of 2-deoxy-2,3-didehydro-N-acetylneuraminic acid modified at the C-4 position. Glycoconj. J. 1993, 10, 40-44.
102. Chong; Pegg; Taylor; von Itzstein. Evidence for a sialosyl cation transition-state complex in the reaction of sialidase from influenza virus. Eur. J. Biochem. 1992, 207, 335-343.
103. Shidmoossavee, F. S.; Watson, J. N.; Bennet, A. J. Chemical insight into the emergence of influenza virus strains that are resistant to Relenza. J. Am. Chem. Soc. 2013, 135, 13254-13257.
104. Cui; Das; Dhawane; Sweeney; Zhang; Chivukula; Iyer. Highly specific and rapid glycan based amperometric detection of influenza viruses. Chem. Sci. 2017, 8, 3628-3634.
105. Li, Z.; Unione, L.; Liu, L.; Lang, Y.; de Vries, R. P.; de Groot, R. J.; Boons, G. J. Synthetic O-acetylated sialosides and their acetamido-deoxy analogues as probes for coronaviral hemagglutinin-esterase recognition. J. Am. Chem. Soc. 2022, 144, 424-435.
106. Morais, G. R.; Oliveira, R. S.; Falconer, R. A. Selective synthesis of Neu5 Ac2en and its oxazoline derivative using BF3·Et2O. Tetrahedron Lett. 2009, 50, 1642-1644.
107. von Itzstein; Jin; Wu; Chandler. A convenient method for the introduction of nitrogen and sulfur at C-4 on a sialic acid analogue. Carbohydr. Res. 1993, 244, 181-185.
108. Lu, Y.; Gervay-Hague, J. Synthesis of C-4 and C-7 triazole analogs of zanamivir as multivalent sialic acid containing scaffolds. Carbohydr. Res. 2007, 342, 1636-1650.
109. Ciccotosto, S.; von Itzstein, M. Synthesis of methyl 5-acetamido-3,4,5-trideoxy-4-guanidinyl-D-glycero-D-galacto-2-nonulopyranosidonic acid (4-deoxy-4-guanidino-Neu5Acα2Me). Tetrahedron Lett. 1995, 36, 5405-5408.
110. Simonsen, K. B.; Ayida, B. K.; Vourloumis, D.; Takahashi, M.; Winters, G. C.; Barluenga, S.; Qamar, S.; Shandrick, S.; Zhao, Q.; Hermann, T. Novel paromamine derivatives exploring shallow-groove recognition of ribosomal-decoding-site RNA. Chembiochem 2002, 3, 1223-1228.
111. Ren, B.; Wang, M.; Liu, J.; Ge, J.; Zhang, X.; Dong, H. Zemplén transesterification: a name reaction that has misled us for 90 years. Green Chem. 2015, 17, 1390-1394.
112. Yu, H.; Yu, H.; Karpel, R.; Chen, X. Chemoenzymatic synthesis of CMP-sialic acid derivatives by a one-pot two-enzyme system: comparison of substrate flexibility of three microbial CMP-sialic acid synthetases. Bioorg. Med. Chem. 2004, 12, 6427-6435.
113. Yu, H.; Chokhawala, H.; Karpel, R.; Yu, H.; Wu, B.; Zhang, J.; Zhang, Y.; Jia, Q.; Chen, X. A multifunctional Pasteurella multocida sialyltransferase: a powerful tool for the synthesis of sialoside libraries. J. Am. Chem. Soc. 2005, 127, 17618-17619.
114. Ding, L.; Zhao, C.; Qu, J.; Li, Y.; Sugiarto, G.; Yu, H.; Wang, J.; Chen, X. A Photobacterium sp. alpha2-6-sialyltransferase (Psp2,6ST) mutant with an increased expression level and improved activities in sialylating Tn antigens. Carbohydr. Res. 2015, 408, 127-133.
115. Baker, T. J.; Luedtke, N. W.; Tor, Y.; Goodman, M. Synthesis and anti-HIV activity of guanidinoglycosides. J. Org. Chem. 2000, 65, 9054-9058.
116. Lau, K.; Thon, V.; Yu, H.; Ding, L.; Chen, Y.; Muthana, M. M.; Wong, D.; Huang, R.; Chen, X. Highly efficient chemoenzymatic synthesis of betal-4-linked galactosides with promiscuous bacterial betal-4-galactosyltransferases. Chem. Commun. 2010, 46, 6066-6068.
117. Baker, B. R.; Joseph, J. P.; Schaub, R. E.; Williams, J. H. Puromycin. Synthetic studies. V. 6-Dimethylamino-9-(2′-acetamino-β-D-glucopyranosyl) purine. J. Org. Chem. 1954, 19, 1786-1792.
118. Sabesan, S. Synthesis and neuraminidase inhibition studies of 4-azido, amino, and acetamido substituted sialosides. Bioorg. Med. Chem. Lett. 1994, 4, 2457-2460.
119. Sabesan, S.; Neira, S.; Wasserman, Z. Structural and functional-group tuning in the design of neuraminidase inhibitors. Carbohydr. Res. 1995, 267, 239-261.
120. Yu, H.; Zeng, J.; Li, Y.; Thon, V.; Shi, B.; Chen, X. Effective one-pot multienzyme (OPME) synthesis of monotreme milk oligosaccharides and other sialosides containing 4-O-acetyl sialic acid. Org. Biomol. Chem. 2016, 14, 8586-8597.
121. Connor, R. J.; Kawaoka, Y.; Webster, R. G.; Paulson, J. C. Receptor specificity in human, avian, and equine H2 and H3 influenza virus isolates. Virology 1994, 205, 17-23.
122. Rogers, G. N.; D'Souza, B. L. Receptor binding properties of human and animal H₁ influenza virus isolates. Virology 1989, 173, 317-322.
123. Li, Y.; Cao, H.; Yu, H.; Chen, Y.; Lau, K.; Qu, J.; Thon, V.; Sugiarto, G.; Chen, X. Identifying selective inhibitors against the human cytosolic sialidase NEU2 by substrate specificity studies. Mol. Biosyst. 2011, 7, 1060-1072.
124. Tasnima, N.; Yu, H.; Li, Y.; Santra, A.; Chen, X. Chemoenzymatic synthesis of para-nitrophenol (pNP)-tagged alpha2-8-sialosides and high-throughput substrate specificity studies of alpha2-8-sialidases. Org. Biomol. Chem. 2016, 15, 160-167.
125. Kim, J. H.; Resende, R.; Wennekes, T.; Chen, H. M.; Bance, N.; Buchini, S.; Watts, A. G.; Pilling, P.; Streltsov, V. A.; Petric, M.; Liggins, R.; Barrett, S.; McKimm-Breschkin, J. L.; Niikura, M.; Withers, S. G. Mechanism-based covalent neuraminidase inhibitors with broad-spectrum influenza antiviral activity. Science 2013, 340, 71-75.
126. Ferraris, O.; Kessler, N.; Lina, B. Sensitivity of influenza viruses to zanamivir and oseltamivir: a study performed on viruses circulating in France prior to the introduction of neuraminidase inhibitors in clinical practice. Antiviral Res. 2005, 68, 43-48.
127. Nishikawa, T.; Shimizu, K.; Tanaka, T.; Kuroda, K.; Takayama, T.; Yamamoto, T.; Hanada, N.; Hamada, Y. Bacterial neuraminidase rescues influenza virus replication from inhibition by a neuraminidase inhibitor. PLOS One 2012, 7, e45371.
128. Wetherall, N. T.; Trivedi, T.; Zeller, J.; Hodges-Savola, C.; McKimm-Breschkin, J. L.; Zambon, M.; Hayden, F. G. Evaluation of neuraminidase enzyme assays using different substrates to measure susceptibility of influenza virus clinical isolates to neuraminidase inhibitors: report of the neuraminidase inhibitor susceptibility network. J. Clin. Microbiol. 2003, 41, 742-750.
129. Keil, J. M.; Rafn, G. R.; Turan, I. M.; Aljohani, M. A.; Sahebjam-Atabaki, R.; Sun, X. L. Sialidase inhibitors with different mechanisms. J. Med. Chem. 2022, 65, 13574-13593.
130. McKimm-Breschkin, J. L.; Colman, P. M.; Jin, B.; Krippner, G. Y.; McDonald, M.; Reece, P. A.; Tucker, S. P.; Waddington, L.; Watson, K. G.; Wu, W. Y. Tethered neuraminidase inhibitors that bind an influenza virus: a first step towards a diagnostic method for influenza. Angew. Chem. Int. Ed. Engl. 2003, 42, 3118-3121.
131. Tai, S. H.; Agafitei, O.; Gao, Z.; Liggins, R.; Petric, M.; Withers, S. G.; Niikura, M. Difluorosialic acids, potent novel influenza virus neuraminidase inhibitors, induce fewer drug resistance-associated neuraminidase mutations than does oseltamivir. Virus Res. 2015, 210, 126-132.

What is claimed is:

1. A compound according to Formula I: 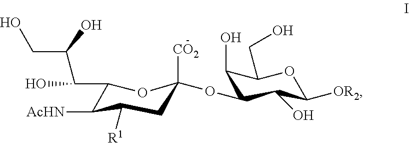
or a pharmaceutically acceptable salt thereof, wherein:
R¹ is selected from the group consisting of NH₂, NHAc, guanidine, and N3; and
R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, 3GlcNAcβ1-3Galβ1-4GlcβProNHCbz, 4GlcNAcβ1-3Galβ1-4GlcβProNHCbz, ProNH-Biotin, and ProNH-PEG4-biotin.

2. The compound of claim 1, wherein the compound is: 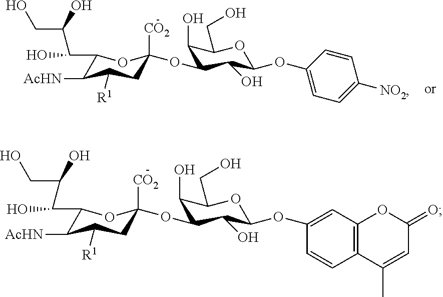
or a pharmaceutically acceptable salt thereof; wherein R¹ is selected from the group consisting of NH₂, NHAc, guanidine, and N₃.

3. The compound of claim 1, wherein R¹ is NH₂.

4. The compound of claim 1, wherein R¹ is guanidine.

5. A compound of Formula II: 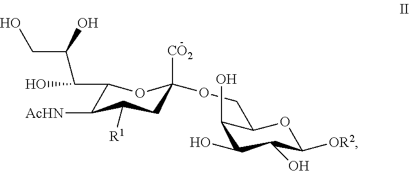
or a pharmaceutically acceptable salt thereof, wherein:
R¹ is selected from the group consisting of NH₂, NHAc, guanidine, and N3; and
R² is selected from the group consisting of 4-nitrophenyl, 4-methylumbelliferyl, 4GlcβProNHCbz, 4GlcNAcβProNHCbz, 3GlcNAcαProNHCbz, 3GlcNAcβProNHCbz, 3GalNAcαProNHCbz, 3GalNAcβProNHCbz, ProNH-Biotin, and ProNH-PEG4-biotin.

6. The compound of claim 5, wherein the compound is: 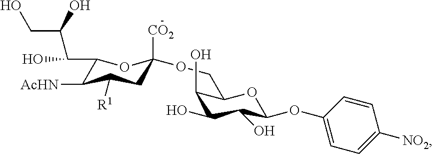
or a pharmaceutically acceptable salt thereof, wherein R¹ is selected from the group consisting of NH₂, NHAc, guanidine, and N₃.

7. The compound of claim 5, wherein the compound is: 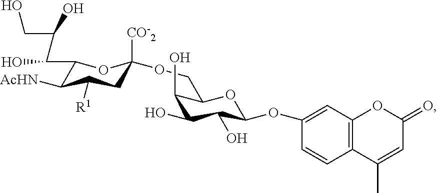
or a pharmaceutically acceptable salt thereof, wherein R¹ is selected from the group consisting of NH₂, NHAc, guanidine, and N₃.

8. The compound of claim 5, wherein R¹ is NH₂.

9. The compound of claim 5, wherein R¹ is guanidine.

10. A biotinylated compound, or pharmaceutically acceptable salt thereof, selected from the group consisting of: 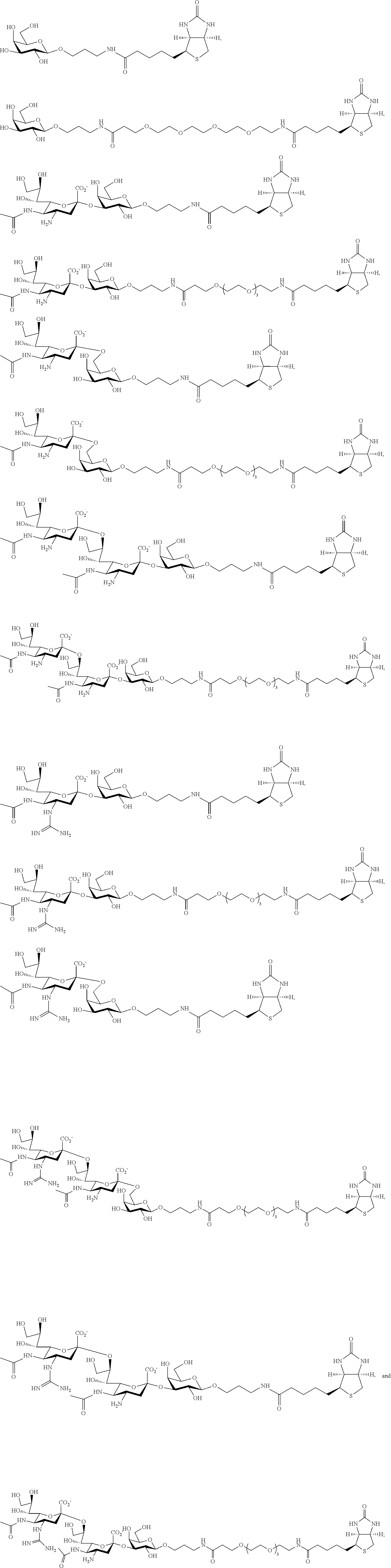
a 4-methylumbelliferyl (MU)-tagged sialoside, or pharmaceutically acceptable salt thereof, selected from the group consisting of: 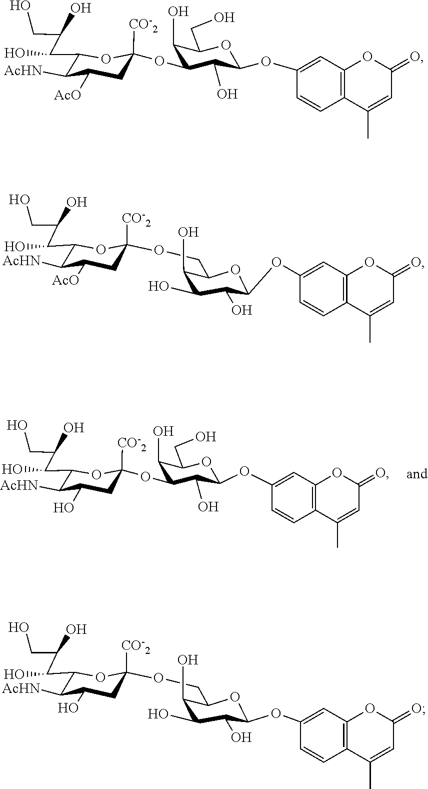
an α2-3-linked 4NH₂Neu5 Ac-sialoside, or pharmaceutically acceptable salt thereof, selected from the group consisting of: 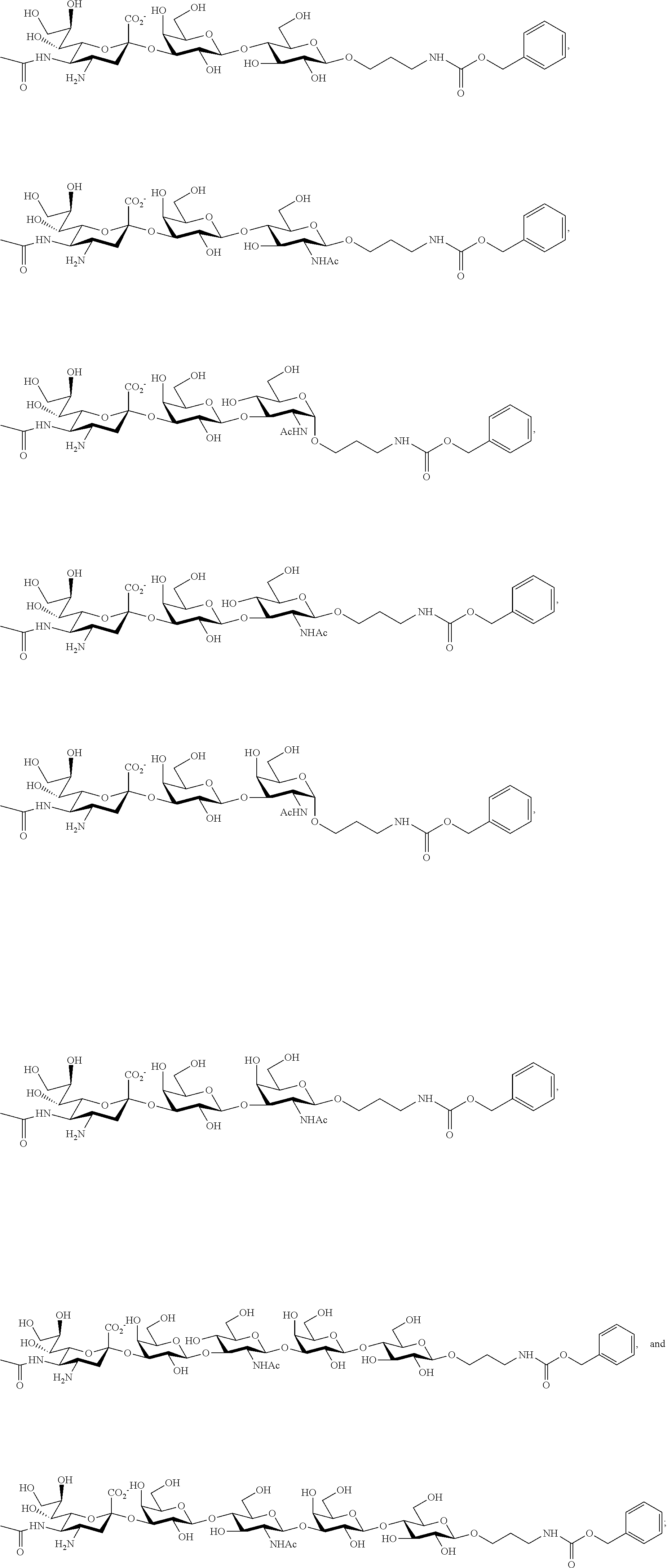
or
an α2-6-linked 4NH₂Neu5Ac-sialoside, or pharmaceutically acceptable salt thereof, selected from the group consisting of:

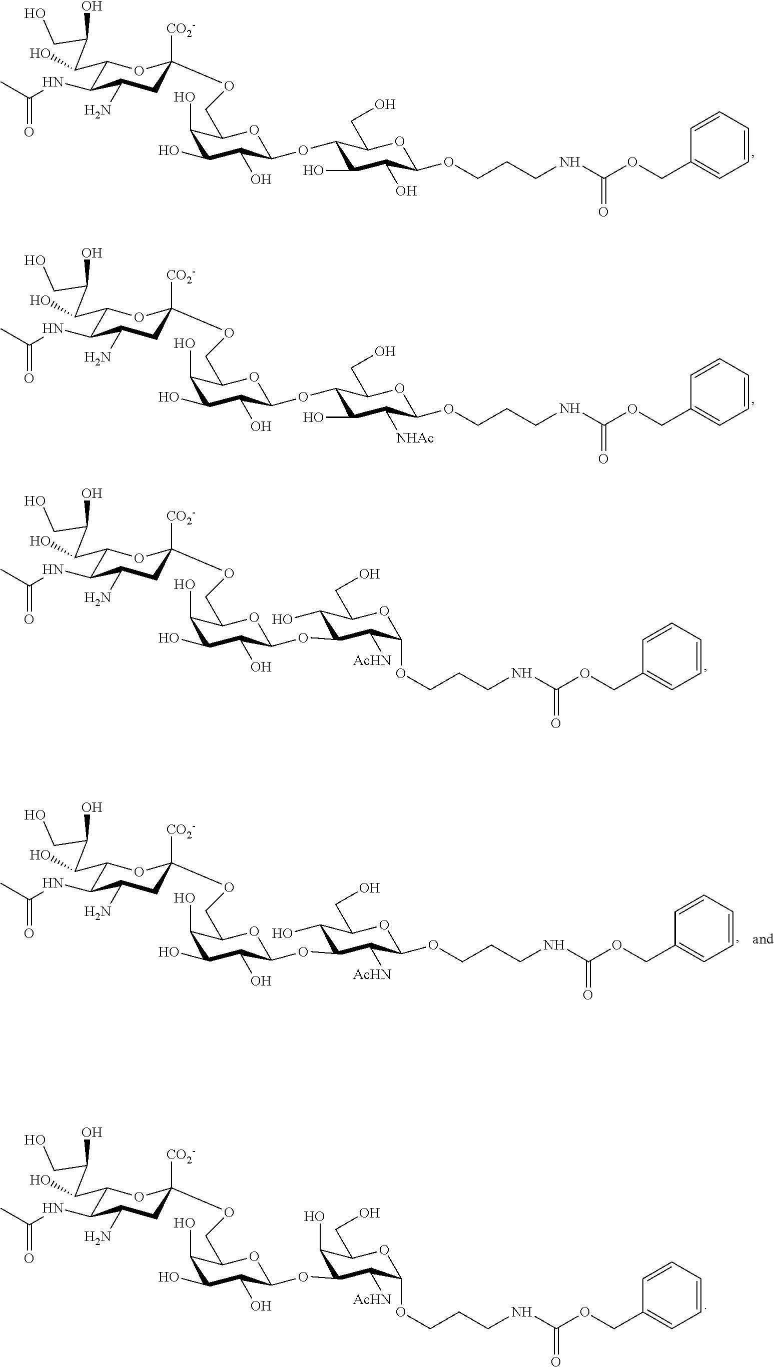

11. A method of inhibiting a neuraminidase, comprising contacting a neuraminidase with a compound of claim 1.

12. The method of claim 17, wherein the neuraminidase is an influenza A virus neuraminidase.

13. The method of claim 17, wherein the neuraminidase is an influenza B virus neuraminidase.

14. A kit for determining a presence of neuraminidases, comprising a compound, or a pharmaceutically acceptable salt thereof, according to claim 1.


SEQUENCE LISTING (11 sequences)

SEQ ID NO: 1 - protein, 227 aa, synthetic construct
  REGION 1..227: note = Description of sequence: catalytically active peptide
EKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILHHKES

SEQ ID NO: 2 - protein, 387 aa, synthetic construct
  REGION 1..387: note = Description of sequence: catalytically active peptide
KTITLYLDPASLPALNQLMDFTQNNEDKTHPRIFGLSRFKIPDNIITQYQNIHFVELKDNRPTEALFTILDQYPGNIELNIHLNIAHSVQLIRPILAYRFKHLDRVSIQQLNLYDDGSDEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAEGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 3 - protein, 307 aa, synthetic construct
  REGION 1..307: note = Description of sequence: catalytically active peptide
NLIICCTPLQVLIAEKIIAKFPHTPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE

SEQ ID NO: 4 - protein, 266 aa, synthetic construct
  REGION 1..266: note = Description of sequence: catalytically active peptide
DKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLVDKLAAALE

SEQ ID NO: 5 - protein, 487 aa, synthetic construct
  REGION 1..487: note = Description of sequence: catalytically active peptide
CNNSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWA

SEQ ID NO: 6 - protein, 482 aa, synthetic construct
  REGION 1..482: note = Description of sequence: catalytically active peptide
CNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 7 - protein, 257 aa, synthetic construct
  REGION 1..257: note = Description of sequence: catalytically active peptide
KKVIIAGNGPSLKEIDYSRLPNDFDVFRCNQFYFEDKYYLGKKCKAVFYNPSLFFEQYYTLKHLIQNEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLMSNFIIQEKNNYTKDILIPSSEAYGKFSKNIN

SEQ ID NO: 8 - protein, 668 aa, synthetic construct
  REGION 1..668: note = Description of sequence: catalytically active peptide
NELNYGQLSISPIFQGGSYQLNNKSIDISPLLLDKLSGDSQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQLIFQSGDSTQANYFRIPTLYTLSSGRVLSSIDARYGGTHDSKSKINIATSYSDDNGKTWSEPIFAMKFNDYEEQLVYWPRDNKLKNSQISGSASFIDSSIVEDKKSGKTILLADVMPAGIGNNNANKADSGFKEINGHYYLKLKKNGDNDFRYTVRENGVVYDETTNKPTNYTINDKYEVLEGGKSLTVEQYSVDFDSGSLRERHNGKQVPMNVFYKDSLFKVTPTNYIAMTTSQNRGESWEQFKLLPPFLGEKHNGTYLCPGQGLALKSSNRLIFATYTSGELTYLISDDSGQTWKKSSASIPFENATAEAQMVELRDGVIRTFFRTTTGKIAYMTSRDSGETWSEVSYIDGIQQTSYGTQVSAIKYSQLIDGKEAVILSTPNSRSGRKGGQLVVGLVNKEDDSIDWKYHYDIDLPSYGYAYSAITELPNHHIGVLFEKYDSWSRNELHLSNVVQYIDLEINDLTK

SEQ ID NO: 9 - protein, 739 aa, synthetic construct
  REGION 1..739: note = Description of sequence: catalytically active peptide
KKNIKQYVTLGTVVVLSAFVANSVAAQETETSEVSTPELVQPVAPTTSISEVQHKSGNSSEVTVQPRTVETTVKDPSSTAEETLVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNAVTGHLIYTANDTTGSNYFRIPVLYTFSNGRVFSSIDARYGGTHDFLNKINIATSYSDDNGKTWTKPKLTLAFDDFAPVPLEWPREVGGRDLQISGGATYIDSVIVEKKNKQVLMFADVMPAGVSFREATRKDSGYKQIDGNYYLKLRKQGDTDYNYTIRENGTVYDDRTNRPTEFSVDKNFGIKQNGNYLTVEQYSVSFENNKKTEYRNGTKVHMNIFYKDALFKVVPTNYIAYISSNDHGESWSAPTLLPPIMGLNRNAPYLGPGRGIIESSTGRILIPSYTGKESAFIYSDDNGASWKVKVVPLPSSWSAEAQFVELSPGVIQAYMRTNNGKIAYLTSKDAGTTWSAPEYLKFVSNPSYGTQLSIINYSQLIDGKKAVILSTPNSTNGRKHGQIWIGLINDDNTIDWRYHHDVDYSNYGYSYSTLTELPNHEIGLMFEKFDSWSRNELHMKNVVPYITFKIEDLKKN

SEQ ID NO: 10 - protein, 739 aa, synthetic construct
  REGION 1..739: note = Description of sequence: catalytically active peptide
KKNIKQYVTLGTVVVLSAFVANSVAAQETETSEVSTPKLVQPVAPTTPISEVQPTSDNSSEVTVQPRTVETTVKDPSSTAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNAVTGHLIYTANDTTGSNYFRIPVLYTFSNGRVFSSIDARYGGTHDFLNKINIATSYSDDNGKTWTKPKLTLAFDDFAPVPLEWPREVGGRDLQISGGATYIDSVIVEKKNKQVLMFADVMPAGVSFREATRKDSGYKQIDGNYYLKLRKQGDTDYNYTIRENGTVYDDRTNRPTEFSVDKNFGIKQNGNYLTVEQYSVSFENNKKTEYRNGTKVHMNIFYKDALFKVVPTNYIAYISSNDHGESWSAPTLLPPIMGLNRNAPYLGPGRGIIESSTGRILIPSYTGKESAFIYSDDNGASWKVKVVPLPSSWSAEAQFVELSPGVIQAYMRTNNGKIAYLTSKDAGTTWSAPEYLKFVSNPSYGTQLSIINYSQLIDGKKAVILSTPNSTNGRKHGQIWIGLINDDNTIDWRYHHDVDYSNYGYSYSTLTELPNHEIGLMFEKFDSWSRNELHMKNVVPYITFKIEDLKKN

SEQ ID NO: 11 - protein, 222 aa, synthetic construct
  REGION 1..222: note = Description of sequence: sialic acid aldolase component
TNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAVRQTLPKMYRANGAIYINDIAQLLKEKYFFIPPLKFYLMPTYHSVDIDVKQDLELAEILSNK